(19) United States
(12) Patent Application Publication
(10) Pub. No.: US 2025/0101065 A1
(43) Pub. Date: Mar. 27, 2025

(54) REVERSE PEPTIDES FROM CORONAVIRUS FOR IMMUNOGENIC PURPOSES

(71) Applicant: Emergex Vaccines Holding Limited, Oxfordshire (GB)

(72) Inventors: Thomas Rademacher, Oxfordshire (GB); Xiaofang Huang, Oxfordshire (GB); Richard David Perrins, Oxfordshire (GB); Nicole Bedke, Oxfordshire (GB); Athanasios Papadopoulos, Oxfordshire (GB); Phillip Williams, Oxfordshire (GB)

(21) Appl. No.: 18/546,586

(22) PCT Filed: Feb. 16, 2022
(86) PCT No.: PCT/EP2022/053825

§ 371 Date: Aug. 15, 2023

Related U.S. Application Data
(60) Provisional application No. 63/196,128, filed on Jun. 2, 2021.

(30) Foreign Application Priority Data
Feb. 16, 2021 (GR) 20210100099

(51) Int. Cl. C07K 14/005; A61K 39/00

(52) U.S. Cl. CPC C07K 14/005; A61K 39/00; A61K 2039/55555; C12N 2770/20022; C12N 2770/20034

(57) ABSTRACT

The invention relates to immunogenic peptides encoded by an open reading frame (ORF) encoded by at least part of the genome of a coronavirus in the opposite sense to positive sense RNA capable of translation. The invention also relates to polynucleotides encoding such peptide, the use of such peptides and polynucleotides for the treatment and prevention of viral infection, and complexes comprising the peptides of the invention.

### PRIORITY ENTITLEMENT

This application claims priority from GR20210100099 filed 16 Feb. 2021 and from U.S. 63/196,128 filed 2 Jun. 2021, the contents and elements of which are herein incorporated by reference for all purposes.

### FIELD OF THE INVENTION

The invention relates to immunogenic peptides encoded by an open reading frame (ORF) encoded by at least part of the genome of a coronavirus in the opposite sense to positive sense RNA capable of translation. The invention also relates to polynucleotides encoding such peptide, the use of such peptides and polynucleotides for the treatment and prevention of viral infection, and complexes comprising the peptides of the invention.

### BACKGROUND

Coronaviruses are a group of related viruses that cause diseases in mammals and birds. Symptoms of coronavirus infections vary between species. For instance, coronavirus infection in chickens causes upper respiratory tract disease, whereas coronavirus infections in cows and pigs tend to cause diarrhoea.

In humans, coronaviruses cause respiratory tract infections. The disease caused by infection with some coronaviruses can be mild, such as the common cold. Other coronaviruses cause more serious and potentially fatal disease, such as SARS (SARS-COV-1), MERS (MERS-CoV), and COVID-19 (SARS-COV-2). As the control of outbreaks of coronavirus infection relies on strategies involving accurate testing and/or effective vaccination, the provision of vaccines and tests is highly desirable.

### SUMMARY OF THE INVENTION

The present invention relates to an immunogenic peptide encoded by an open reading frame (ORF) encoded by at least part of the genome of a coronavirus, in the opposite sense to positive sense RNA capable of translation. In other words, the immunogenic peptide is encoded by an open reading frame (ORF) encoded by at least part of the reverse complement of the genome of a coronavirus. The immunogenic peptide comprises an epitope, such as a CD8+T cell epitope, a CD4+T cell epitope, or a B cell epitope. The present invention also relates to a polynucleotide encoding the immunogenic peptide, to the use of such immunogenic peptides and polynucleotides for the treatment and prevention of viral infection, and to complexes comprising the immunogenic peptide.

The present inventors have surprisingly identified a number of immunogenic peptides that are presented by MHC class I molecules on cells infected with the coronavirus SARS-COV-2 and that are encoded by an ORF in the opposite sense to positive sense RNA capable of translation (i.e. by part of the reverse complement of the ORF 1ab polyprotein gene). The ORF is widely conserved amongst SARS-COV-2 strains and is therefore thought to confer a selective advantage to SARS-COV-2. Including one or more immunogenic peptides encoded by the conserved ORF in a pharmaceutical composition may confer protective and/or therapeutic capability against SARS-COV-2. Including one or more immunogenic peptides encoded by the conserved ORF in a pharmaceutical composition may also confer protective and/or therapeutic capability against one or more coronaviruses (or coronavirus strains) other than SARS-COV-2. Including a plurality of such immunogenic peptides (for instance, each capable of binding to a different HLA supertype), in a pharmaceutical composition may provide a broad-spectrum composition that has prophylactically and/or therapeutically effective in individuals having different HLA types. Similarly, protective capability against SARS-COV-2 may be conferred a polynucleotide (e.g. RNA or mRNA) based composition comprising one or more polynucleotides (e.g. RNAs or mRNAs) encoding an immunogenic peptide identified by the present inventors.

The immunogenic peptides identified by the inventors may also have utility in investigation of SARS-COV-2 specific immune responses, and/or immune responses specific for other coronaviruses. For this purpose, an immunogenic peptide of the invention may be provided as a complex comprising the immunogenic peptide bound to an MHC molecule.

The present inventors have also surprisingly identified immunogenic peptides that are presented by MHC class I molecules on cells infected with the endemic common cold coronavirus 229E and that are encoded by an ORF encoded by part of the genome in the opposite sense to positive sense RNA capable of translation (i.e. by part of the reverse complement of the genome).

Accordingly, the present invention provides an immunogenic peptide comprising an epitope from a polypeptide encoded by an open reading frame (ORF) encoded by at least part of the genome of a coronavirus in the opposite sense to positive sense RNA capable of translation.

The present invention further provides:
a polynucleotide (e.g RNA, mRNA) encoding an immunogenic peptide of the invention;
a pharmaceutical composition comprising the immunogenic peptide of the invention or the polynucleotide (e.g. RNA, mRNA) of the invention;
a method of preventing and/or treating a coronaviral infection, comprising administering the pharmaceutical composition the invention to an individual;
the pharmaceutical composition of the invention for use in a method of preventing and/or treating a coronaviral infection in an individual; and
a complex comprising the immunogenic peptide of the invention bound to an MHC molecule.

The invention described herein includes all combinations of aspects and/or preferred features, except where such a combination is clearly impermissible or expressly avoided.

### DESCRIPTION OF THE FIGURES

FIG. 1: Schematic showing the location of an ORF encoded by part of the orflab gene of SARS-COV-2 in the opposite sense to positive sense RNA capable of translation.

FIG. 2: Schematic showing the location of the peptides of SEQ ID NOs: 2 to 12 within an ORF encoded by part of the genome of SARS-COV-2 in the opposite sense to positive sense RNA capable of translation.

FIG. 3: Fragment mass spectra of peptide ALLTCQTIGLCN (SEQ ID NO: 2)

FIG. 4: Fragment mass spectra of peptide LHSRTSFCMVGFS (SEQ ID NO: 3)

FIG. 5: Fragment mass spectra of peptide LSIPCASSDFST (SEQ ID NO: 4)

FIG. 6: Fragment mass spectra of peptide LTCQTIGLCNNLAPF (SEQ ID NO: 5)

FIG. 7: Fragment mass spectra of peptide LYVALLVALLTCQ (SEQ ID NO: 6)

FIG. 8: Fragment mass spectra of peptide QTIGLCNNLAP (SEQ ID NO: 7)

FIG. 9: Fragment mass spectra of peptide RLSIPCASSDF (SEQ ID NO: 8)

FIG. 10: Fragment mass spectra of peptide TCQTIGLCNNLAPFL (SEQ ID NO: 9)

FIG. 11: Fragment mass spectra of peptide TLHSRTSFCMV (SEQ ID NO: 10)

FIG. 12: Fragment mass spectra of peptide VALLTCQTIGLCN (SEQ ID NO: 11)

FIG. 13: Fragment mass spectra of peptide VFTLHSRTSF (SEQ ID NO: 12)

FIG. 14: ORF100 variability in UK SARS-COV-2 strains (Example 3). Graph shows the normalised Shannon entropy for each position in ORF100. Note a value of 0 corresponds to a highly conserved position while 1 represents a position where there is an equal probability of any of the 20 amino acids being found.

FIG. 15: ORF100 variability in SARS-COV-2 strains, irrespective of clade, sampling date, geographic location (Example 4). Graph shows the normalised Shannon entropy for each position in ORF100. Note a value of 0 corresponds to a highly conserved position while 1 represents a position where there is an equal probability of any of the 20 amino acids being found.

FIG. 16: Expansion of FIG. 15.

FIG. 17: Alignment of consensus sequence with early SARS-COV-2 and bat-coronavirus ORF100 sequences

FIG. 18: ELISPOT data from stimulations of PBMCs (from donors EVB00028, EVB00031 and EVB00033) with negative sense SARS-COV-2 peptides.

FIG. 19: Flow cytometry analysis of T cell surface markers after stimulation of PBMCs from EVB00028.

FIG. 20: Flow cytometry analysis of T cell surface markers after stimulation of PBMCs from EVB00033.

FIG. 21: A*2402/VFTLHSRTSF-APC MHC Dextramer staining analysis.

FIG. 22: banding associated with a polynucleotide of the expected size for a polynucleotide encoding ORF100.

FIG. 23: Validation of mass spectra for peptide RLSIPCASSDF (confirmed). A. Fragment mass spectra from cell lysate; B. Fragment mass spectra from synthetic peptides. Arrow refers to some typical ion peak to show the identity in each group.

FIG. 24: Validation of mass spectra for peptide TLHSRTSFCMV (confirmed). A. Fragment mass spectra from cell lysate; B. Fragment mass spectra from synthetic peptides. Arrow refers to some typical ion peak to show the identity in each group. Both groups have same precursor ion peak: A (641.43) and B (641.45). Another matching ion peak is Y6: panel A (344.24) and panel B (335.21). The difference is due to loss of a water molecule and charge+2, hence panel B shows a reduction of 8 by mass over charge.

FIG. 25: Validation of mass spectra for peptide VALLTCQTIGLCN (confirmed). A. Fragment mass spectra from cell lysate; B. Fragment mass spectra from synthetic peptides. Arrow refers to some typical ion peak to show the identity in each group. Comparing to synthetic peptide (panel B), peptide (panel A) in cell lysate shows low abundance which leads to less fragment ion peaks in the region of 800-1200 (m/z). However, there are still signature fragment ion peaks in both panels such as Y4, B8 (see arrows).

FIG. 26: Validation of mass spectra for peptide VFTLHSRTSF (confirmed). A. Fragment mass spectra from cell lysate; B. Fragment mass spectra from synthetic peptides. Arrow refers to some typical ion peak to show the identity in each group.

FIG. 27: Summary of HcoV229E derived peptide in control and infection groups.

FIG. 28: Summary of self-derived peptides in control and infection groups.

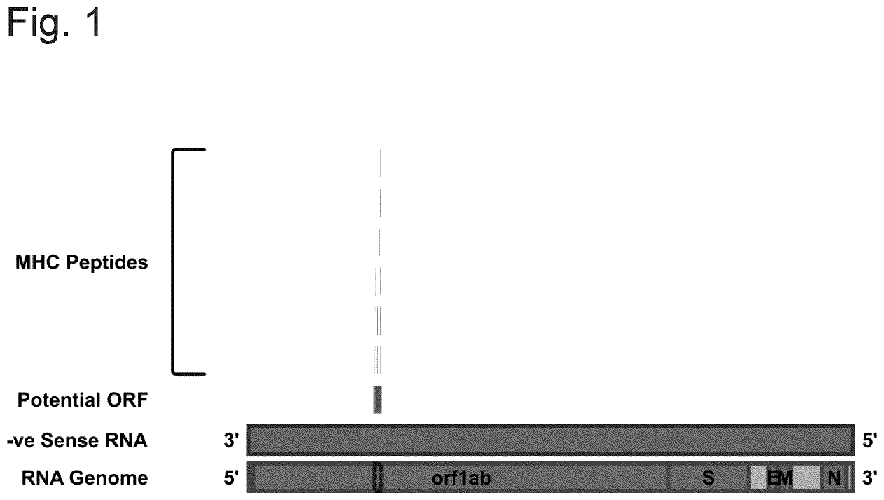

### Description of the Sequence Listing

SEQ ID NO: 1—polynucleotide sequence of an ORF encoded by part of the orflab gene of SARS-COV-2 in the opposite sense to positive sense RNA capable of translation. Each n is independently selected from t and u. In one aspect, each n may, for example, bet. That is, all of the ns may be t. In another aspect, each n may, for example, be u. That is, all of the ns may be u. In a further aspect, some of the ns may be t and some of the ns may be u. That is, the sequence may contain a combination of t and u.
SEQ ID NO: 2—immunogenic peptide encoded by an ORF encoded by part of the orflab gene of SARS-COV-2 in the opposite sense to positive sense RNA capable of translation
SEQ ID NO: 3—immunogenic peptide encoded by an ORF encoded by part of the orflab gene of SARS-COV-2 in the opposite sense to positive sense RNA capable of translation
SEQ ID NO: 4—immunogenic peptide encoded by an ORF encoded by part of the orflab gene of SARS-COV-2 in the opposite sense to positive sense RNA capable of translation
SEQ ID NO: 5—immunogenic peptide encoded by an ORF encoded by part of the orflab gene of SARS-COV-2 in the opposite sense to positive sense RNA capable of translation
SEQ ID NO: 6—immunogenic peptide encoded by an ORF encoded by part of the orflab gene of SARS-COV-2 in the opposite sense to positive sense RNA capable of translation
SEQ ID NO: 7—immunogenic peptide encoded by an ORF encoded by part of the orflab gene of SARS-COV-2 in the opposite sense to positive sense RNA capable of translation
SEQ ID NO: 8—immunogenic peptide encoded by an ORF encoded by part of the orflab gene of SARS-COV-2 in the opposite sense to positive sense RNA capable of translation
SEQ ID NO: 9—immunogenic peptide encoded by an ORF encoded by part of the orflab gene of SARS-COV-2 in the opposite sense to positive sense RNA capable of translation
SEQ ID NO: 10—immunogenic peptide encoded by an ORF encoded by part of the orflab gene of SARS-COV-2 in the opposite sense to positive sense RNA capable of translation
SEQ ID NO: 11—immunogenic peptide encoded by an ORF encoded by part of the orflab gene of SARS-COV-2 in the opposite sense to positive sense RNA capable of translation
SEQ ID NO: 12—immunogenic peptide encoded by an ORF encoded by part of the orflab gene of SARS-COV-2 in the opposite sense to positive sense RNA capable of translation
SEQ ID NO: 13—consensus amino acid sequence encoded by an ORF encoded by part of the orflab gene of SARS-COV-2 strains in the opposite sense to positive sense RNA capable of translation.
SEQ ID NO: 14—amino acid sequence encoded by an ORF encoded by the genome of severe acute respiratory syndrome coronavirus 2 isolate WIV04 (MN996528.1) in the opposite sense to positive sense RNA capable of translation.
SEQ ID NO: 15—amino acid sequence encoded by an ORF encoded by the genome of Wuhan seafood market pneumonia virus isolate Wuhan-Hu-1 (NC045512.2) in the opposite sense to positive sense RNA capable of translation.
SEQ ID NO: 16—amino acid sequence encoded by an ORF encoded by the genome of bat coronavirus isolate RaTG13 (MN996532.1) in the opposite sense to positive sense RNA capable of translation.
SEQ ID NO: 17—amino acid sequence encoded by an ORF encoded by the genome of bat SARS-like coronavirus isolate bat-SL-CoVZXC21 (MG772934.1) in the opposite sense to positive sense RNA capable of translation.
SEQ ID NO: 18—amino acid sequence encoded by an ORF encoded by the genome of bat SARS-like coronavirus isolate bat-SL-CoVZC45 (MG772933.1) in the opposite sense to positive sense RNA capable of translation.
SEQ ID NO: 19—oligo used in the Examples.
SEQ ID NO: 20—oligo used in the Examples.
SEQ ID NO: 21—oligo used in the Examples.
SEQ ID NO: 22—oligo used in the Examples.
SEQ ID NO: 23—amplification primer used in the Examples.
SEQ ID NO: 24—amplification primer used in the Examples.
SEQ ID NO: 25—T7 RNA Polymerase Template Primer used in the Examples. Key: T7 Promoter Kozak Primer for LNSORF100
SEQ ID NO: 26—T7 RNA Polymerase Template Primer used in the Examples. Key: Poly (A) Primer
SEQ ID NOs: 27 to 45—amino acid sequences encoded by the genome of endemic common cold coronavirus 229E (NP_073549.1) in the same sense to positive sense RNA capable of translation.
SEQ ID NO: 46—amino acid sequence encoded by an ORF encoded by the genome of endemic common cold coronavirus (NP_073549.1) in the opposite sense to positive sense RNA capable of translation.
SEQ ID NOs: 57 to 52—amino acid sequences encoded by the genome of endemic common cold coronavirus 229E in the same sense to positive sense RNA capable of translation.
SEQ ID NO: 53—amino acid sequence encoded by an ORF encoded by the genome of endemic common cold coronavirus 229E (NP_073549.1) in the opposite sense to positive sense RNA capable of translation.
SEQ ID NOs: 54 to 62—amino acid sequences encoded by the genome of endemic common cold coronavirus 229E (NP_073549.1) in the same sense to positive sense RNA capable of translation.

### DETAILED DESCRIPTION

Aspects and embodiments of the present invention will now be discussed with reference to the accompanying figures. Further aspects and embodiments will be apparent to those skilled in the art. All documents mentioned in this text are incorporated herein by reference.

Single stranded RNA (ssRNA) viruses are classified as positive-sense or negative-sense depending on the sense or polarity of their genomic RNA. In a negative-sense ssRNA virus, the negative-sense (3′ to 5′) genomic RNA is complementary to the mRNA and must generally be converted to a positive-sense RNA by RNA polymerase before translation. In a positive-sense ssRNA virus, such as a coronavirus, the positive-sense (5′ to 3′) genomic RNA can also serve as mRNA and can be translated into protein in the host cell. That is, replication of a negative-sense ssRNA virus generally occurs following transcription of the genome to form an mRNA that is complementary to the genome, and translation of the mRNA. Replication of a positive-sense ssRNA virus generally occurs following translation the genomic RNA that doubles as mRNA.

Traditionally, it has been considered that the negative sense RNA of ssRNA viruses is non-coding. However, non-canonical translation may be possible. It appears that the negative-sense genomic RNA of certain negative-sense ssRNA viruses, such as influenza virus, may actually be capable of translation in the 5′ to 3′ direction, yielding different gene products to those obtained via canonical translation (i.e. 3′ to 5′ transcription forming a complementary mRNA, and translation of the mRNA 5′ to 3′). Similarly, RNA complementary to the genome of positive-sense ssRNA viruses may actually be translatable.

Coronaviruses are positive-sense ssRNA viruses. The present inventors have obtained data that demonstrates that polypeptides obtained from non-canonical translation in coronaviruses may comprise peptides that are recognised by the immune system of an individual infected with the coronavirus. For instance, a polypeptide obtained from non-canonical translation in a coronavirus may comprise a peptide that is capable of presentation by an MHC class I molecule and of recognition by a T cell receptor (TCR) present on a CD8+T cell. In other words, a polypeptide obtained from non-canonical translation in a coronavirus may comprise a peptide that is a CD8+ T cell epitope. A polypeptide obtained from non-canonical translation in a coronavirus may, for instance, comprise a peptide that is capable of presentation by an MHC class II molecule and of recognition by a T cell receptor (TCR) present on a CD4+T cell. In other words, a polypeptide obtained from non-canonical translation in a coronavirus may comprise a peptide that is a CD4+T cell epitope. A polypeptide obtained from non-canonical translation in a coronavirus may, for instance, comprise a peptide that is capable of specifically binding to an antibody, and/or of recognition by a B cell receptor (BCR) present on a B cell. In other words, a polypeptide obtained from non-canonical translation in a coronavirus may comprise a peptide that is a B cell epitope. Any such immunogenic peptide may be administered to an individual, for instance in a pharmaceutical composition, in order to induce an immune response against the coronavirus. In this way, prophylaxis and/or therapy may be achieved.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a polynucleotide” includes “polynucleotides”, reference to “an immunogenic peptide” includes two or more such immunogenic peptides, and the like.

In general, the term “comprising” is intended to mean including but not limited to. For example, the phrase “immunogenic peptide comprising an epitope” should be interpreted to mean that the immunogenic peptide contains an epitope, but that the immunogenic peptide may also contain additional amino acids.

In some aspects of the disclosure, the word “comprising” is replaced with the phrase “consisting of”. The term “consisting of” is intended to be limiting. For example, the phrase “immunogenic peptide consisting of an epitope” should be understood to mean that the immunogenic peptide contains an epitope and no additional amino acids.

For the purpose of this disclosure, in order to determine the percent identity of two sequences (such as two polynucleotide or two amino acid sequences), the sequences are aligned for optimal comparison purposes (e.g., gaps can be introduced in a first sequence for optimal alignment with a second sequence). The nucleotide/amino acid residues at nucleotide/amino acid positions are then compared. When a position in the first sequence is occupied by the same nucleotide or amino acid residue as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. For standard molecular biology techniques, see Sambrook, J., Russel, D.W. Molecular Cloning, A Laboratory Manual. 3 ed. 2001, Cold Spring Harbor, New York: Cold Spring Harbor Laboratory Press, which is incorporated herein by reference in its entirety.

### Immunogenic Peptides of the Invention

The present invention provides an immunogenic peptide comprising an epitope from a polypeptide encoded by an open reading frame (ORF) encoded by at least part of the genome of a coronavirus in the opposite sense to positive sense RNA capable of translation. This immunogenic peptide has a number of benefits which will become apparent from the discussion below. The key benefits are summarised here.

Firstly, the immunogenic peptide comprises an epitope. The epitope may, for example, be a CD8+ T cell epitope, such as a peptide set out in one of SEQ ID NOs: 2 to 12, 46 and 53 and newly identified by the inventors. The immunogenic peptide is therefore capable of stimulating an immune response against a coronavirus or multiple coronaviruses. The immune response may, for example be a cellular immune response (e.g. a CD8+ T cell response) when the epitope is a CD8+ T cell epitope. CD8+ cytotoxic T lymphocytes (CTLs) mediate viral clearance via their cytotoxic activity against infected cells. Stimulating cellular immunity may therefore provide a beneficial defence against coronavirus infection. The immune response may, for example be a humoral immune response (e.g. a B cell response or antibody response) when the epitope is a B cell epitope. Humoral immune responses mediate neutralisation and/or clearance of virus via antibody binding. Stimulating humoral immunity may therefore provide a beneficial defence against coronavirus infection. The immune response may, for example be a helper T cell response (e.g. a CD4+ T cell response) when the epitope is a CD4+T cell epitope. T helper lymphocytes “help” the activity of other immune cells by secreting immune mediators, such as cytokines. Stimulating a helper T cell response may therefore facilitate or augment cellular and/or humoral immune responses against the coronavirus, providing a beneficial defence against the virus.

Secondly, the ORF encoding the polypeptide from which the epitope comprised in the immunogenic peptide is derived may be conserved between multiple coronaviruses or coronavirus strains. Thus, the polypeptide may be conserved between multiple coronaviruses or coronavirus strains. The epitope itself may therefore be conserved between multiple coronaviruses or coronavirus strains. For instance, the ORF, the polypeptide and/or the epitope may be conserved between different strains of SARS-COV-2. A pharmaceutical composition comprising the immunogenic peptide may therefore provide cross-protection and/or therapeutic benefit against a plurality of different strains of SARS-COV-2. In this way, a pharmaceutical composition comprising the immunogenic peptide is suitable for providing broad-spectrum prophylaxis or treatment against SARS-COV-2, countering the problems caused by the mutations such as those recently reported in the art. In other words, a single pharmaceutical composition comprising the immunogenic peptide may induce protective and/or therapeutic immunity against a wide variety of existing and emerging SARS-COV-2 strains. This may reduce or eliminate the need for a new vaccine or treatment to be developed as SARS-COV-2 mutates. The ORF and/or the epitope may be conserved between different human coronaviruses. A pharmaceutical composition comprising the immunogenic peptide may therefore provide cross-protection and/or therapeutic benefit against a plurality of different human coronaviruses. In this way, a pharmaceutical composition comprising the immunogenic peptide (or a polynucleotide encoding the immunogenic peptide) is suitable for providing broad-spectrum prophylaxis against and/or treatment for various human coronaviruses, including existing and emerging human coronaviruses.

The ORF, the polypeptide and/or the epitope may be conserved between human coronaviruses and coronaviruses of other animals. In this way, a pharmaceutical composition comprising the immunogenic peptide of the invention may prevent animal coronaviruses becoming established in the human population. In other words, as well as protecting against existing human coronaviruses, a pharmaceutical composition comprising the immunogenic peptide of the invention may prevent animal coronaviruses crossing the species divide. This may prevent the emergence of further pandemic human coronaviruses.

Thirdly, the immunogenic peptide may be capable of binding to different HLA supertypes. Including in a pharmaceutical composition multiple immunogenic peptides of the invention each comprising an epitope capable of binding to a different HLA supertypes results in a pharmaceutical composition that is effective in individuals having different HLA types. In this way, a single pharmaceutical composition can be used to confer protection against a coronavirus in a large proportion of the human population. This provides a cost-effective means of controlling the incidence and spread of virus infection. Similarly, a single therapeutic composition can be used to treat a coronavirus in a large proportion of the human population. This provides a cost-effective means of treating coronavirus infection.

Fourthly, the polypeptide encoded by the ORF encoding the immunogenic peptide may enhance the fitness of the coronavirus in humans. In other words, the polypeptide may confer a selective advantage on the coronavirus by which it is encoded. A pharmaceutical composition comprising the immunogenic peptide of the invention may therefore target epitopes associated with a coronavirus conferred with a selective advantage. The pharmaceutical composition of the invention may therefore be well-designed to provide effective prophylaxis or treatment against emergent and/or potentially pandemic strains of a coronavirus. The pharmaceutical composition of the invention may therefore be well-designed to provide effective prophylaxis or treatment against endemic strains of a coronavirus, or seasonal strains of a coronavirus.

Fifthly, the immunogenic peptide may be attached to a nanoparticle, for example a gold nanoparticle. As described in more detail below, attachment to a nanoparticle reduces or eliminates the need to include an adjuvant in a pharmaceutical (e.g. vaccine) composition comprising the immunogenic peptide. Thus, a pharmaceutical composition comprising the immunogenic peptide of the invention is less likely to cause adverse clinical effects upon administration to an individual.

### Immunogenic Peptide

An immunogenic peptide is a peptide that is capable of eliciting an immune response. The immunogenic peptide of the invention comprises an epitope from a polypeptide encoded by an ORF encoded by at least part of the genome of a coronavirus in the opposite sense to its positive sense RNA capable of translation.

The epitope may, for example, be a CD8+ T cell epitope, a CD4+T cell epitope, or a B cell epitope. The epitope may, for example, be a nested epitope, such as a CD8+ T cell epitope that comprises a CD4+T cell epitope. Preferably, the epitope is a CD8+ T cell epitope.

A CD8+ T cell epitope is a peptide that is capable of (i) presentation by a class I MHC molecule and (ii) recognition by a T cell receptor (TCR) present on a CD8+T cell. Preferably, recognition by the TCR results in activation of the CD8+T cell. CD8+T cell activation may lead to increased proliferation, cytokine production and/or cytotoxic effects. Typically, a CD8+T cell epitope is around 9 amino acids in length. A CD8+ T cell epitope may though be shorter or longer. For example, a CD8+ T cell epitope may be about 8, 9, 10, 11, 12, 13, 14 or 15 amino acids in length. A CD8+ T cell epitope may be about 8 to 15, 9 to 14, 10 to 13, or 11 to 12 amino acids in length.

A CD4+T cell epitope is a peptide that is capable of (i) presentation by a class II MHC molecule and (ii) recognition by a T cell receptor (TCR) present on a CD4+T cell. Preferably, recognition by the TCR results in activation of the CD4+T cell. CD4+T cell activation may lead to increased proliferation and/or cytokine production. Typically, a CD4+T cell epitope is around 12 to 15 amino acids in length. A CD4+T cell epitope may though be shorter or longer. For example, a CD4+T cell epitope may be about 8, 9, 10, 11, 12, 13, 14, 15, 16, 17, 18 or 19 amino acids in length. A CD8+ T cell epitope may be about 8 to 19, 9 to 18, 10 to 17, 11 to 16, 12 to 14, or 13 to 15 amino acids in length.

A B cell epitope is a peptide that is capable of recognition by a B cell receptor (BCR) present on a B cell, or by an antibody. The B cell epitope may, therefore, be an antibody-binding epitope. Recognition by the BCR may, for example, result in activation and/or maturation of the B cell. B cell activation may lead to increased proliferation, and/or antibody production. The B cell epitope may be a linear epitope, i.e. an epitope that is defined by the primary amino acid sequence encoded by the ORF. Alternatively, the epitope may be a conformational epitope, i.e. an epitope that is defined by the conformational structure of a native protein encoded by the ORF. In this case, the epitope may be continuous (i.e. the components that interact with the antibody are situated next to each other sequentially on the protein) or discontinuous (i.e. the components that interact with the antibody are situated on disparate parts of the protein, which are brought close to each other in the folded native protein structure). Typically, the B cell epitope or antibody-binding epitope is around 5 to 20 amino acids in length. For example, a B cell epitope may be about 6, 7, 8, 9, 10, 11, 12, 13, 14, 15, 16, 17, 18, 19 or 20 amino acids in length. A B cell epitope may be about 6 to 19, 7 to 18, 8 to 17, 9 to 16, 10 to 15, 11 to 14 or 12 to 13 amino acids in length.

The epitope comprised in the immunogenic peptide is from a polypeptide encoded by an ORF encoded by at least part of the genome of a coronavirus in the opposite sense to positive sense RNA capable of translation. In other words, the epitope may, in nature, be comprised in or form part of a polypeptide encoded by an ORF encoded by at least part of the genome of a coronavirus in the opposite sense to positive sense RNA capable of translation. That is, the epitope may be comprised in or form part of a polypeptide encoded by an ORF encoded by at least part of the reverse complement of the genome of a coronavirus. The polypeptide may be expressed on the surface of a coronavirus, or within a coronavirus virion. Expression of the polypeptide by the virus may be transient i.e. the polypeptide may only be expressed by the coronavirus at certain points in its lifecycle and/or under certain environmental conditions. Alternatively, the coronavirus may have sustained expression of the polypeptide. The polypeptide may be a structural peptide or a functional peptide, such as a peptide that is involved in the metabolism or replication of the coronavirus. In some cases, however, the purpose and/or function of the polypeptide may be unknown.

The polypeptide that gives rise to the epitope may enhance the fitness of the coronavirus in humans. For example, the polypeptide may improve the survival and/or replication of the virus in a human host cell. Accordingly, the polypeptide may confer a selective advantage on the coronavirus. In other words, expression of the polypeptide may enable the coronavirus to survive and/or reproduce better than an coronavirus that does not express the polypeptide. In this case, the evolutionary selective process may select for viruses in which at least part of the genome encodes, in the opposite sense to positive sense RNA capable of translation, an ORF encoding the polypeptide. The ORF (and, consequently, the polypeptide and/or epitope) may therefore be conserved between different coronaviruses, or different strains of a given coronavirus.

The ORF, the polypeptide and/or the epitope may, for example, conserved between two or more (such as three or more, four or more, five or more, six or more, seven or more, eight or more, nine or more, 10 or more, 20 or more, 50 or more, 100 or more, 250 or more, 500 or more, 750 or more, or 1000 or more) human coronaviruses. For instance, the ORF, the polypeptide and/or the epitope may be conserved between two or more different strains of SARS-COV-2. The ORF, the polypeptide and/or the epitope may, for example, be conserved between SARS-CoV-2 and one or more other human coronavirus. For example, the ORF, the polypeptide and/or the epitope may be conserved between SARS-COV-2 and one or more of (a) SARS-COV-1, (b) 229E, (c) NL63, (d) OC43, (e) HKU1 and (f) MERS-COV. The ORF, the polypeptide and/or the epitope may, for example, be conserved between SARS-COV-2 and: (a); (b); (c); (d); (e); (f); (a) and (b); (a) and (c); (a) and (d); (a) and (e); (a) and (f); (b) and (c); (b) and (d); (b) and (e); (b) and (f); (c) and (d); (c) and (e); (c) and (f); (d) and (e); (d) and (f); (e) and (f); (a), (b) and (c); (a), (b) and (d); (a), (b) and (e); (a), (b) and (f); (a), (c) and (d); (a), (c) and (e); (a), (c) and (f); (a), (d) and (e); (a), (d) and (f); (a), (e) and (f); (b), (c) and (d); (b), (c) and (e); (b), (c) and (f); (b), (d) and (e); (b), (d) and (f); (b), (e) and (f); (c), (d) and (e); (c), (d) and (f); (c), (e), and (f); (d), (e) and (f); (a), (b), (c) and, (d); (a), (b), (c) and (e); (a), (b), (c) and (f); (a), (b), (d) and (e); (a), (b), (d) and (f); (a), (b), (e) and (f); (a), (c), (d) and (e); (a), (c), (d) and (f); (a), (c), (e) and (f); (a), (d), (e) and (f); (b), (c), (d) and (e); (b), (c), (d) and (f); (b), (c), (e) and (f); (b), (d), (e) and (f); (c), (d), (e) and (f); (a), (b), (c), (d) and (e); (a), (b), (c), (d) and (f); (a), (b), (c), (e) and (f); (a), (b), (d), (e) and (f); (a), (c), (d), (e) and (f); (b), (c), (d), (e) and (f); or (a), (b), (c), (d), (e) and (f). The ORF, the polypeptide and/or the epitope may be conserved between SARS-COV-2 and one or more endemic common cold coronaviruses. For example, the ORF, the polypeptide and/or the epitope may be conserved between SARS-COV-2 and one or more of (i)229E, (ii) NL63, (iii) OC43 and (iv) HKU1. ORF, the polypeptide and/or the epitope may, for example, be conserved between SARS-COV-2 and (i); (ii); (iii); (iv); (i) and (ii); (i) and (iii); (i) and (iv); (ii) and (iii); (ii) and (iv); (iii) and (iv); (i), (ii) and (iii); (i), (ii) and (iv); (i), (iii) and (iv); (ii), (iii) and (iv); or (i), (ii), (iii) and (iv). The ORF, the polypeptide and/or the epitope may be conserved between 229E and one or more other human coronaviruses.

The ORF, the polypeptide and/or the epitope may, for example, be conserved between one or more (e.g. two or more, three or more, four or more, five or more, six or more, seven or more, eight or more, nine or more, 10 or more, 20 or more, 50 or more, 100 or more, 250 or more, 500 or more, 750 or more, or 1000 or more) human coronaviruses and one or more (e.g. two or more, three or more, four or more, five or more, six or more, seven or more, eight or more, nine or more, 10 or more, 20 or more, 50 or more, 100 or more, 250 or more, 500 or more, 750 or more, or 1000 or more) animal coronaviruses. For instance, the ORF, the polypeptide and/or the epitope may, for example, be conserved between one or more SARS-COV-2 viruses and one or more animal coronaviruses. The one or more animal coronaviruses may comprise a coronavirus capable of infecting bats.

The ORF, the polypeptide and/or the epitope may, for example, be conserved between two or more animal coronaviruses. For example, the ORF, the polypeptide and/or the epitope may be conserved between three or more, four or more, five or more, six or more, seven or more, eight or more, nine or more, 10 or more, 20 or more, 50 or more, 100 or more, 250 or more, 500 or more, 750 or more, or 1000 or more animal coronaviruses. The two or more animal coronaviruses may comprise coronaviruses capable of infecting the same species of animal. The two or more animal coronaviruses may comprise coronaviruses capable of infecting different species of animal. The two or more animal coronaviruses may comprise coronaviruses capable of infecting bats.

The ORF (and, consequently, the polypeptide and/or epitope) is conserved between two or more different coronaviruses if there is at least 20% (such as at least 30%, at least 40%, at least 50%, at least 60%, at least 70%, at least 75%, at least 80%, at least 90%, at least 95%, at least 98% or at least 99%) identity between the ORF encoded by at least part of the genome of each virus, in the opposite sense to positive sense RNA capable of translation.

The epitope comprised in the immunogenic peptide may be present in a polypeptide encoded by a predicted ORF of about 100 codons in length in one or more of (a) SARS-COV-1, (b)229E, (c) NL63, (d) OC43, (e) HKU1 and (f) MERS-COV. In this case, presence of the CD8+ T cell epitope in a predicted ORF of about 100 codons in length in one or more of (a) to (f) may indicate that a pharmaceutical composition comprising the immunogenic peptide provides protection and/or treatment against infection with the one or more of (a) to (f). The epitope comprised in the immunogenic peptide may be present in a polypeptide encoded by an ORF of about 100 codons in length in SARS-COV-2, and in a polypeptide encoded by a predicted ORF of about 100 codons in length in one or more of (a) SARS-COV-1, (b)229E, (c) NL63, (d) OC43, (e) HKU1 and (f) MERS-COV. In this case, presence of the epitope in a predicted ORF of about 100 codons in length in one or more of (a) to (f) may indicate that a pharmaceutical composition comprising the immunogenic peptide provides cross-protection for and/or therapeutic benefit against SARS-COV-2 and the one or more of (a) to (f).

The immunogenic peptide may comprise only one epitope from a polypeptide encoded by an ORF encoded by at least part of the genome of a coronavirus in the opposite sense to positive sense RNA capable of translation. Alternatively, the immunogenic peptide may comprise two or more, such as three or more, four or more, five or more, six or more, seven or more, eight or more, nine or more, ten or more, fifteen or more, or twenty or more such epitopes. When the immunogenic peptide comprises two or more epitopes each from a polypeptide encoded by an ORF encoded by at least part of the genome of a coronavirus in the opposite sense to positive sense RNA capable of translation, each of the epitopes may be independently selected from a CD8+ T cell epitope, a CD4+T cell epitope, and a B cell epitope. For instance, the immunogenic peptide may comprise two or more CD8+ T cell epitopes each from a polypeptide encoded by an ORF encoded by at least part of the genome of a coronavirus in the opposite sense to positive sense RNA capable of translation. The immunogenic peptide may comprise two or more CD4+T cell epitopes each from a polypeptide encoded by an ORF encoded by at least part of the genome of a coronavirus in the opposite sense to positive sense RNA capable of translation. The immunogenic peptide may comprise two or more B cell epitopes each from a polypeptide encoded by an ORF encoded by at least part of the genome of a coronavirus in the opposite sense to positive sense RNA capable of translation. The immunogenic peptide may comprise one or more (e.g. two or more) CD8+ T cell epitopes each from a polypeptide encoded by an ORF encoded by at least part of the genome of a coronavirus in the opposite sense to positive sense RNA capable of translation, and one or more (e.g. two or more) CD4+T cell epitopes each from a polypeptide encoded by an ORF encoded by at least part of the genome of a coronavirus in the opposite sense to positive sense RNA capable of translation. The immunogenic peptide may comprise one or more (e.g. two or more) CD8+ T cell epitopes each from a polypeptide encoded by an ORF encoded by at least part of the genome of a coronavirus in the opposite sense to positive sense RNA capable of translation, and one or more (e.g. two or more) B cell epitopes each from a polypeptide encoded by an ORF encoded by at least part of the genome of a coronavirus in the opposite sense to positive sense RNA capable of translation. The immunogenic peptide may comprise one or more (e.g. two or more) CD4+T cell epitopes each from a polypeptide encoded by an ORF encoded by at least part of the genome of a coronavirus in the opposite sense to positive sense RNA capable of translation, and one or more (e.g. two or more) B cell epitopes each from a polypeptide encoded by an ORF encoded by at least part of the genome of a coronavirus in the opposite sense to positive sense RNA capable of translation.

As well as the epitope(s) from a polypeptide encoded by an ORF encoded by at least part of the genome of a coronavirus in the opposite sense to positive sense RNA capable of translation, the immunogenic peptide may comprise one or more other CD8+ T cell epitopes, one or more other CD4+T cell epitopes and/or one or more other B cell epitopes. For example, the immunogenic peptide may comprise one or more, such as two or more, three or more, four or more, five or more, ten or more, fifteen or more, or twenty or more CD8+ T cell epitopes that are not a CD8+ T cell epitope from a polypeptide encoded by an ORF encoded by at least part of the genome of a coronavirus in the opposite sense to positive sense RNA capable of translation. The immunogenic peptide may comprise one or more, such as two or more, three or more, four or more, five or more, ten or more, fifteen or more, or twenty or more CD4+T cell epitopes that are not a CD4+T cell epitope from a polypeptide encoded by an ORF encoded by at least part of the genome of a coronavirus in the opposite sense to positive sense RNA capable of translation. The immunogenic peptide may comprise one or more, such as two or more, three or more, four or more, five or more, ten or more, fifteen or more, or twenty or more B cell epitopes that are not a B cell epitope from a polypeptide encoded by an ORF encoded by at least part of the genome of a coronavirus in the opposite sense to positive sense RNA capable of translation.

Many coronaviral B cell epitopes, CD4+T cell epitopes and CD8+ T cell epitopes are known in the art. Methods for identifying B cell epitopes, CD4+T cell epitopes and CD8+T cell epitopes are known in the art. Epitope mapping methods include X-ray co-crystallography, array-based oligo-peptide scanning (sometimes called overlapping peptide scan or pepscan analysis), site-directed mutagenesis, high throughput mutagenesis mapping, hydrogen-deuterium exchange, crosslinking coupled mass spectrometry, phage display and limited proteolysis. MHC motif prediction methodologies may also be used. CD8+ T cell epitopes presented by coronavirus-infected cells can be identified in order to directly identify CD8+ T cell epitopes, as described below.

Preferably, the immunogenic peptide comprises or consists of one or more of the sequences set out in SEQ ID NOs: 2 to 12, 46 and 53, or a variant thereof having at least 50% sequence identity to the relevant sequence selected from SEQ ID NOs: 2 to 12, 46 and 53. The variant may, for example, have at least 55%, at least 60%, at least 65%, at least 70%, at least 75%, at least 80%, at least 85%, at least 90%, at least 95%, at least 97%, at least 98%, at least 99%, or 100% sequence identity to the relevant sequence selected from SEQ ID NOs: 2 to 12, 46 and 53. Each of SEQ ID NOs: 2 to 12 represents a CD8+ T cell epitope from a polypeptide encoded by an ORF encoded by at least part of the genome of SARS-Cov-2 in the opposite sense to positive sense RNA capable of translation. Each of SEQ ID NOs: 46 and 53 represents a CD8+ T cell epitope from a polypeptide encoded by an ORF encoded by at least part of the genome of 229E in the opposite sense to positive sense RNA capable of translation. SEQ ID NOs: 2 to 12 are set out in Table 1. Table 1 also shows the HLA supertype to which each of SEQ ID NOs: 2 to 12 binds. The HLA binding shown in Table 1 represents the amalgamated results from (i) analysis of traditional motif patterns and (ii) the output of two binding prediction programs (MHCflurry and PSSMHCpan, selecting for KDs <=1 μM).

TABLE 1

| SEQ | | |
|---|---|---|
| ID | | |
| NO: | Sequence | HLA binding |
| 2 | ALLTCQTIGLCN | A2, A203/A2,A69 |
| 3 | LHSRTSFCMVGFS | B61, B41, B47 |
| 4 | LSIPCASSDFST | A2, A203/A2, A69, B57 |
| 5 | LTCQTIGLCNNLAPF | A1, A2, A203/A2, A24, A69, B57, |
| | | B58, B62 |
| 6 | LYVALLVALLTCQ | A2, A203/A2, A69, B35 |
| 7 | QTIGLCNNLAP | A2, A11, A26, A29, A31, A68, |
| | | B41, B50 |
| 8 | RLSIPCASSDF | A1, A3, A11, A24, A26, A30, B7, |
| | | B41, B58 B62, B72 |
| 9 | TCQTIGLCNNLAPFL | A2 (preliminary result) |
| 10 | TLHSRTSFCMV | A2, A203/A2, A26, A29, A30, |
| | | A68, A69, B41, B58, B62 |
| 11 | VALLTCQTIGLCN | A2, A203/A2, A69, B61, B41, B47 |
| 12 | VFTLHSRTSF | A23, A24, A29, A30, A31, A33, |
| | | B62, B75, B49, B53, C4, C16 |

SEQ ID Nos: 46 and 53 are set out in Table 15.

The immunogenic peptide may comprise or consist of all of the sequences set out in SEQ ID NOs: 2 to 12. The immunogenic peptide may comprise or consist of a variant of each of SEQ ID NOs: 2 to 12, wherein the variant has at least 50% sequence identity to the relevant sequence selected from SEQ ID NOs: 2 to 12. The variant may, for example, have at least 55%, at least 60%, at least 65%, at least 70%, at least 75%, at least 80%, at least 85%, at least 90%, at least 95%, at least 97%, at least 98%, or 99% sequence identity to the relevant sequence selected from SEQ ID NOs: 2 to 12. The immunogenic peptide may comprise or consist of both of the sequences set out in SEQ ID NOs: 46 and 53. The immunogenic peptide may comprise or consist of a variant of each of SEQ ID NOs: 46 and 53, wherein the variant has at least 50% sequence identity to the relevant sequence selected from SEQ ID NOs: 46 and 53. The variant may, for example, have at least 55%, at least 60%, at least 65%, at least 70%, at least 75%, at least 80%, at least 85%, at least 90%, at least 95%, at least 97%, at least 98%, or 99% sequence identity to the relevant sequence selected from SEQ ID NOs: 46 and 53.

The immunogenic peptide may comprise a polypeptide comprising the sequence: MSPTTSVVFTLHSRTSFCMVGFSTTSSETGFRSSQARLSIPCASSDFSTSNEFDVSTGFVLQ RQRIHQVFGLYVALLVALLTCQTIGLCNNLAPFLKEGV (SEQ ID NO: 13) or a variant having at least 50% sequence identity thereto. The variant may, for example, have at least 55%, at least 60%, at least 65%, at least 70%, at least 75%, at least 80%, at least 85%, at least 90%, at least 95%, at least 97%, at least 98%, or 99% sequence identity to SEQ ID NO: 13.

The immunogenic peptide may comprise a polypeptide comprising the sequence of any of SEQ ID NOs: 14 to 18, or a variant having at least 50% sequence identity thereto. The variant may, for example, have at least 55%, at least 60%, at least 65%, at least 70%, at least 75%, at least 80%, at least 85%, at least 90%, at least 95%, at least 97%, at least 98%, or 99% sequence identity to the respective sequence selected from SEQ ID NOs: 14 to 18.

Any of the immunogenic peptides described herein may contain any number of amino acids, i.e. be of any length. For example, the immunogenic peptide may be about 8, about 9, about 10, about 15, about 20, about 25, about 30, about 35, about 40, about 45, about 50, about 55, about 60, about 65, about 70, about 75, about 80, about 85, about 90, about 95 or about 100 amino acids in length. For example, the immunogenic peptide may be about 8 to about 100, about 9 to about 95, about 10 to about 90, about 15 to about 85, about 20 to about 80, about 25 to about 75, about 30 to about 70, about 35 to about 65, about 40 to about 60, about 45 to about 55, or about 50 amino acids in length. Typically, the immunogenic peptide is about 8 to about 30, 35 or 40 amino acids in length, such as about 9 to about 29, about 10 to about 28, about 11 to about 27, about 12 to about 26, about 13 to about 25, about 13 to about 24, about 14 to about 23, about 15 to about 22, about 16 to about 21, about 17 to about 20, or about 18 to about 29 amino acids in length.

Any of the immunogenic peptides described herein may be chemically derived from a polypeptide antigen, for example by proteolytic cleavage. More typically, the immunogenic peptide may be synthesised using methods well known in the art.

The term “peptide” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol.159, 3230-3237, which is incorporated herein by reference in its entirety. This approach involves making pseudopeptides containing changes involving the backbone, and not the orientation of side chains. Meziere et al (1997) show that, at least for MHC class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N- or C-terminus so as to help reduce susceptibility to exoproteolytic digestion. For example, the N-terminal amino group of the peptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH2 may be modified to —NH(Me) or —N(Me)₂).

The term “peptide” also includes peptide variants that increase or decrease the half-life of the peptide in vivo. Examples of analogues capable of increasing the half-life of peptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

The immunogenic peptide may, for example, interact with at least two different HLA supertypes. This allows the immunogenic peptide to elicit a CD8+ T cell response in a greater proportion of individuals or cell samples therefrom. The immunogenic peptide may, for example, interact with at least two, at least three, at least four, at least five, at least six, at least 7, at least 8, at least 9 or at least 10 different HLA supertypes. Each immunogenic peptide may, for example, interact with two or more of A1, A10, All, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, and C9, or any other HLA supertype known in the art, in any combination.

### CD8. T Cell Epitope

The immunogenic peptide of the invention comprises an epitope from a polypeptide encoded by an ORF encoded by at least part of the genome of a coronavirus in the opposite sense to positive sense RNA capable of translation. In other words, the immunogenic peptide of the invention comprises an epitope from a polypeptide encoded by an ORF encoded by at least part of the reverse complement of the genome of a coronavirus. The epitope is preferably a CD8+T cell epitope. Accordingly, the immunogenic peptide of the invention may comprise a CD8+T cell epitope from a polypeptide encoded by an ORF encoded by at least part of the reverse complement of the genome of a coronavirus.

CD8+ T cell epitopes presented by coronavirus-infected cells can be identified in order to directly identify CD8+ T cell epitopes for inclusion in the immunogenic polypeptide. This is an efficient and logical method which can be used alone or to confirm the utility of potential CD8+T cell epitopes identified by MHC motif prediction methodologies.

To perform the method, cells are infected with a coronavirus (such as SARS-COV-2) and maintained in culture for a period of around 72 hours at a temperature of around 37° C. Following culture, the cells are then harvested and washed. Next, the cells are lysed, for instance by homogenisation and freezing/thawing in buffer. Lysis buffer may, for example, contain around 1% NP40. Lysates may be cleared of cell debris, for instance by centrifugation at, for example, around 3000 rpm for around 10 minutes. MHC/peptide complexes may then be isolated from the lysates. Numerous ways of doing so are known in the art. For example, immunoaffinity chromatography may be used to isolate MHC/peptide complexes, as described in WO 2019/186199, which is incorporated herein by reference in its entirety. An immunoproteomic protocol may alternatively be used. In any case, the peptide-containing fractions may then be cleaned up, for instance using high performance liquid chromatography. The peptide-containing fractions are analysed by mass spectrometry to identify the sequences of the fractions. The acquired spectral data can then be searched against all databased proteins for the coronavirus to identify peptide sequences associated with the coronavirus. Synthetic peptides may optionally be made according to the identified sequences and subjected to mass spectrometry to confirm their identity to the peptides in the peptide-containing fractions.

In this method, and similar methods known in the art, any type of cells may be infected with any type of coronavirus. Coronaviruses are described in detail below. The cells may be antigen presenting cells (APCs). The cells may be hepatoma cells such as HepG2 cells, EBV-transformed lymphoblastoid B cells such as JY cells, or lymphoblasts such as T2 cells.

The direct identification of CD8+ T cell epitopes presented by ssRNA virus-infected cells is advantageous compared to MHC motif prediction methodologies. The immune epitope database (IEDB; http://www.iedb.org) is generated by motif prediction methods, and not functional methods, and contains numerous predicted HLA-specific ssRNA virus T cell epitopes, including some shared epitopes with high MHC binding scores and limited CTL characterization. As both dominant and subdominant epitopes may be presented by virus-infected cells, it is difficult to sort out the dominance hierarchies of naturally presented epitopes using the database. Thus, it is not clear from the immune epitope database alone which of the listed epitopes may be expected to efficiently induce a CD8+ T cell response when included in a pharmaceutical composition. The direct identification method set out above provides a mechanism for confirming the utility of the epitopes.

Pharmaceutical compositions based on epitopes presented by coronavirus-infected cells, may be superior to vaccines based on a viral protein subunit or a motif predicted epitope. Protein processing by the immune system is likely to alter native viral epitopes. Basing a pharmaceutical composition on peptides demonstrated to be presented by infected cells removes this source of uncertainty, because the peptides have already undergone protein processing.

### Polypeptide

The immunogenic peptide of the invention comprises an epitope from a polypeptide. The polypeptide is encoded by an ORF encoded by at least part of the genome of a coronavirus in the opposite sense to positive sense RNA capable of translation. That is, the polypeptide is encoded by an ORF encoded by at least part of the reverse complement of the genome of a coronavirus.

The polypeptide may contain any number of amino acids, i.e. be of any length. Typically, the polypeptide is about 100 amino acids in length, such as about 90 to about 110, about 91 to about 109, about 92 to about 108, about 93 to about 107, about 94 to about 106, about 95 to about 105, about 96 to about 104, about 97 to about 103, about 98 to about 102, or about 99 to about 101 amino acids in length.

The polypeptide may consist of the epitope comprised in the immunogenic peptide of the invention. Alternatively, the polypeptide may comprise the epitope comprised in the immunogenic peptide of the invention. The polypeptide may, for example, comprise two or more different epitopes each comprised in a different immunogenic peptide of the invention. For instance, the polypeptide may comprise two or more different CD8+ T cell epitopes each comprised in a different immunogenic peptide of the invention. For example the polypeptide may comprise two or more different CD8+ T cell epitopes each selected from: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; and SEQ ID NO: 12. The polypeptide may, for example, comprise three or more, four or more, five or more, six or more, seven or more, eight or more, nine or more, ten or more, or 11 or more different CD8+ T cell epitopes each selected from: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; and SEQ ID NO: 12.

The polypeptide may, for example, the comprise two or more different CD8+T cell epitopes each selected from: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 46; and SEQ ID NO: 53. The polypeptide may, for example, comprise three or more, four or more, five or more, six or more, seven or more, eight or more, nine or more, ten or more, or 11 or more different CD8+ T cell epitopes each selected from: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 46; and SEQ ID NO: 53. The polypeptide may, for example, comprise two different CD8+T cell epitopes each selected from: SEQ ID NO: 46 and SEQ ID NO: 53.

The polypeptide may comprise or consist of all of the sequences set out in SEQ ID NOs: 2 to 12. The polypeptide may comprise or consist of all of the sequences set out in SEQ ID NOs: 2 to 12, 46 and 53. The polypeptide may comprise or consist of a variant of one or more, or all, of SEQ ID NOs: 2 to 12, wherein the variant has at least 50% sequence identity to the relevant sequence selected from SEQ ID NOs: 2 to 12. The variant may, for example, have at least 55%, at least 60%, at least 65%, at least 70%, at least 75%, at least 80%, at least 85%, at least 90%, at least 95%, at least 97%, at least 98%, or 99% sequence identity to the relevant sequence selected from SEQ ID NOs: 2 to 12. The polypeptide may comprise or consist of a variant of one or more, or all, of SEQ ID NOs: 46 and 53, wherein the variant has at least 50% sequence identity to the relevant sequence selected from SEQ ID NOs: 46 and 53. The variant may, for example, have at least 55%, at least 60%, at least 65%, at least 70%, at least 75%, at least 80%, at least 85%, at least 90%, at least 95%, at least 97%, at least 98%, or 99% sequence identity to the relevant sequence selected from SEQ ID NOs: 46 and 53.

The polypeptide may, for example, comprise the sequence MSPTTSVVFTLHSRTSFCMVGFSTTSSETGFRSSQARLSIPCASSDFSTSNEFDVSTGFVLQ RQRIHQVFGLYVALLVALLTCQTIGLCNNLAPFLKEGV (SEQ ID NO: 13) or a variant having at least 50% sequence identity thereto. The variant may, for example, have at least 55%, at least 60%, at least 65%, at least 70%, at least 75%, at least 80%, at least 85%, at least 90%, at least 95%, at least 97%, at least 98%, or 99% sequence identity to SEQ ID NO: 13.

The polypeptide may comprise a polypeptide comprising the sequence of any of SEQ ID NOs: 14 to 18, or a variant having at least 50% sequence identity thereto. The variant may, for example, have at least 55%, at least 60%, at least 65%, at least 70%, at least 75%, at least 80%, at least 85%, at least 90%, at least 95%, at least 97%, at least 98%, or 99% sequence identity to the respective sequence selected from SEQ ID NOs: 14 to 18.

The polypeptide may comprise or consist of both of the sequences set out in SEQ ID NOs: 46 and 52. The polypeptide may comprise or consist of a variant of one or more, or both, of SEQ ID NOs: 46 and 53, wherein the variant has at least 50% sequence identity to the relevant sequence selected from SEQ ID NOs: 46 and 53. The variant may, for example, have at least 55%, at least 60%, at least 65%, at least 70%, at least 75%, at least 80%, at least 85%, at least 90%, at least 95%, at least 97%, at least 98%, or 99% sequence identity to the relevant sequence selected from SEQ ID NOs: 46 and 53.

### Open Reading Frame (ORF)

The epitope comprised in the immunogenic peptide is derived from a polypeptide encoded by an ORF encoded by at least part of the genome of a coronavirus in the opposite sense to positive sense RNA capable of translation. In other words, the epitope comprised in the immunogenic peptide is from a polypeptide encoded by an ORF encoded by at least part of the reverse complement of the genome of a coronavirus.

A reading frame is a grouping of three successive nucleotides in a nucleic acid sequence, such as an RNA sequence, that constitutes the codons for the amino acids encoded by the nucleic acid sequence. An ORF (open reading frame) is the part of a reading frame that can be translated. An ORF is a continuous stretch of codons that contain a start codon and a stop codon. Within the ORF, an initiation codon (e.g. ATG) may serve as an initiation site for translation of the RNA into protein.

In the present invention, the ORF is encoded by at least part of the genome of the coronavirus, in the opposite sense to positive sense RNA capable of translation. Coronaviruses are positive-sense ssRNA viruses. In a positive-sense ssRNA virus, the viral genomic RNA is the same sense as the mRNA and can be translated without intervening transcription i.e. the genomic RNA doubles as mRNA. Here, the ORF is encoded in the opposite sense to the viral genomic RNA and, therefore, the mRNA. Accordingly, the ORF may be negative sense. That is, the ORF may require the genomic RNA to be transcribed before it is capable of translation.

The ORF may, for example, be encoded by the whole of the genome of the coronavirus in the opposite sense to positive sense RNA capable of translation. Alternatively, the ORF may be encoded by only some of the genome of the coronavirus in the opposite sense to positive sense RNA capable of translation. For instance, the ORF may be encoded by about 1% to about 99%, such as about 2% to about 98%, about 3% to about 97%, about 5% to about 95%, about 10% to about 90%, about 15% to about 85%, about 20% to about 80%, about 25% to about 75%, about 30% to about 70%, about 40% to about 60%, or about 50% of the genome of the coronavirus in the opposite sense to positive sense RNA capable of translation.

The ORF may be of any length. Preferably, the ORF is about 100 codons in length, such as about 90 to about 110, about 91 to about 109, about 92 to about 108, about 93 to about 107, about 94 to about 106, about 95 to about 105, about 96 to about 104, about 97 to about 103, about 98 to about 102, or about 99 to about 101 codons in length. The ORF may, for example, be at least 90, such as at least 91, at least 92, at least, 93, at least 94, at least 95, at least 96, at least 97, at least 98, at least 99, at least 100, at least 101, at least 102, at least 103, at least 104, at least 105, at least 106, at least 107, at least 108, or at least 109, at least 110 codons in length. The ORF may be about 300 nucleotides in length, such as about 270 to about 330, about 273 to about 327, about 276 to about 324, about 279 to about 321, about 282 to about 318, about 285 to about 315, about 288 to about 312, about 291 to about 309, about 294 to about 306, or about 297 to about 303 nucleotides in length. The ORF may, for example, be at least 270, such as at least 273, at least 275, at least, 278, at least 291, at least 294, at least 297, at least 300, least 303, at least 306, at least 309, at least 312, at least 315, at least 318, at least 321, at least 324, or at least 327, at least 330 nucleotides in length.

The ORF may, for example, be encoded by at least part of the genome of a SARS-COV-2 virus in the opposite sense to positive sense RNA capable of translation. The portion of the positive sense genome that corresponds to the ORF may, for example, encode pp1a/pp1ab/nsp3. The portion of the positive sense genome that corresponds to the ORF may, for example, comprise or consist of residues 6189 to 6489 of the complete genome sequence of severe acute respiratory syndrome coronavirus 2 isolate Wuhan-Hu-1 (GenBank accession no. NC_045512).

The ORF may, for example, comprise the sequence:

angncnccnacaacnncggnagnnnncacannacacncaagaacgnennnengnanggnaggannnnccacnacnncnncag agacnggnnnnagancnncgcaggcaagannanccanncccngcgcgnccncngacnncagnacancaaacgaannngangn nncaacnggnnnngngcnccaaagacaacgnanacaccaggnannnggnnnanacgnggcnnnannagnngcanngnnaac angccaaacaanaggnnnangnaacaannnagcnccnnncnnaaaagagggngngnag (SEQ ID NO: 1) or a variant having at least 50% sequence identity thereto. In SEQ ID NO: 1 each n is independently selected from t and u. In one aspect, each n may, for example, bet. That is, all of the ns may be t. In another aspect, each n may, for example, be u. That is, all of the ns may be u. In a further aspect, some of the ns may be t and some of the ns may be u. That is, the sequence may contain a combination of t and u. The variant may, for example, have at least 55%, at least 60%, at least 65%, at least 70%, at least 75%, at least 80%, at least 85%, at least 90%, at least 95%, at least 97%, at least 98%, or 99% sequence identity to SEQ ID NO: 1.

The ORF may, for example, encode a polypeptide comprising the sequence: MSPTTSVVFTLHSRTSFCMVGFSTTSSETGFRSSQARLSIPCASSDFSTSNEFDVSTGFVLQ RQRIHQVFGLYVALLVALLTCQTIGLCNNLAPFLKEGV (SEQ ID NO: 13) or a variant having at least 50% sequence identity thereto. The variant may, for example, have at least 55%, at least 60%, at least 65%, at least 70%, at least 75%, at least 80%, at least 85%, at least 90%, at least 95%, at least 97%, at least 98%, or 99% sequence identity to SEQ ID NO: 13.

The ORF may, for example, encode a polypeptide may comprise a polypeptide comprising the sequence of any of SEQ ID NOs: 14 to 18, or a variant having at least 50% sequence identity thereto. The variant may, for example, have at least 55%, at least 60%, at least 65%, at least 70%, at least 75%, at least 80%, at least 85%, at least 90%, at least 95%, at least 97%, at least 98%, or 99% sequence identity to the respective sequence selected from SEQ ID NOs: 14 to 18.

As mentioned above, the ORF encoding the polypeptide that gives rise to the epitope comprised in the immunogenic peptide may be conserved between different coronaviruses or coronavirus strains. For instance, the ORF may be conserved between different strains of SARS-CoV-2, or between SARS-COV-2 and one or more other human coronavirus such as (a) SARS-CoV-1, (b)229E, (c) NL63, (d) OC43, (e) HKU1 and/or (f) MERS-COV. The ORF may be conserved between a human coronavirus (such as SARS-COV-2) and one or more animal coronaviruses. The ORF may be conserved between different strains of 229E, or between 229E and one or more other human coronavirus such as (a) SARS-COV-1, (b) SARS-COV-2, (c) NL63, (d) OC43, (e) HKU1 and/or (f) MERS-COV. The ORF may be conserved between two or more different coronaviruses or coronavirus strains if there is at least 50% (such as at least 60%, at least 70%, at least 75%, at least 80%, at least 90%, at least 95%, at least 98% or at least 99%) identity between the ORF encoded by at least part of the genome of each virus in the opposite sense to positive sense RNA capable of translation. For example, the ORF may be conserved between two or more different coronaviruses or coronavirus strains if there is about 75% (such as about 70% to about 80%, e.g. about 71%, about 72%, about 73%, about 74%, about 76%, about 77%, about 78%, or about 79%) identity between the ORF encoded by at least part of the genome of each virus, in the opposite sense to positive sense RNA capable of translation. The polypeptide encoded by the ORF may enhance the fitness of the coronavirus in humans, i.e. confer a selective advantage on the coronavirus.

The ORF may be conserved between two or more (such as three or more, four or more, five or more, six or more, seven or more, eight or more, nine or more, 10 or more, 20 or more, 50 or more, 100 or more, 250 or more, 500 or more, 750 or more, or 1000 or more) coronaviruses or coronavirus strains. For example, the ORF may be conserved between two or more SARS-COV-2 strains. The ORF may be conserved between two or more of: (a) one or more SARS-COV-2 viruses; (b) one or more SARS-COV-1 viruses; (c) one or more 229E viruses; (d) one or more NL63 viruses; (e) one or more OC43 viruses; (f) one or more HKU1 viruses; and (g) one or more MERS-COV viruses.

The polypeptide encoded by the ORF, and/or the epitope it includes, may similarly be conserved between two or more coronaviruses or coronavirus strains. A pharmaceutical composition (e.g. vaccine) comprising the immunogenic peptide of the invention may therefore be capable of providing cross-protection against multiple coronaviruses or coronavirus strains. A single pharmaceutical composition comprising the immunogenic peptide of the invention may therefore be able to induce protective immunity against a variety of existing and emerging coronaviruses. A single pharmaceutical composition comprising the immunogenic peptide of the invention may be able to induce protective immunity against a variety of endemic and seasonal coronaviruses. For example, in one aspect, the polypeptide and/or epitope may be conserved between two or more SARS-COV-2 viruses. In this case, a pharmaceutical composition (e.g. vaccine) comprising the immunogenic peptide of the invention may be capable of providing protection against two or more strains of SARS-COV-2. In another aspect, the polypeptide and/or epitope may be conserved between a SARS-COV-2 virus and one or more of (a) SARS-CoV-1, (b)229E, (c) NL63, (d) OC43, (e) HKU1 and (f) MERS-COV. The polypeptide and/or the epitope may, for example, be conserved between SARS-COV-2 and: (a); (b); (c); (d); (e); (f); (a) and (b); (a) and (c); (a) and (d); (a) and (e); (a) and (f); (b) and (c); (b) and (d); (b) and (e); (b) and (f); (c) and (d); (c) and (e); (c) and (f); (d) and (e); (d) and (f); (e) and (f); (a), (b) and (c); (a), (b) and (d); (a), (b) and (e); (a), (b) and (f); (a), (c) and (d); (a), (c) and (e); (a), (c) and (f); (a), (d) and (e); (a), (d) and (f); (a), (e) and (f); (b), (c) and (d); (b), (c) and (e); (b), (c) and (f); (b), (d) and (e); (b), (d) and (f); (b), (e) and (f); (c), (d) and (e); (c), (d) and (f); (c), (e), and (f); (d), (e) and (f); (a), (b), (c) and, (d); (a), (b), (c) and (e); (a), (b), (c) and (f); (a), (b), (d) and (e); (a), (b), (d) and (f); (a), (b), (e) and (f); (a), (c), (d) and (e); (a), (c), (d) and (f); (a), (c), (e) and (f); (a), (d), (e) and (f); (b), (c), (d) and (e); (b), (c), (d) and (f); (b), (c), (e) and (f); (b), (d), (e) and (f); (c), (d), (e) and (f); (a), (b), (c), (d) and (e); (a), (b), (c), (d) and (f); (a), (b), (c), (e) and (f); (a), (b), (d), (e) and (f); (a), (c), (d), (e) and (f); (b), (c), (d), (e) and (f); or (a), (b), (c), (d), (e) and (f). In this case, a pharmaceutical composition (e.g. vaccine) comprising the immunogenic peptide of the invention may be able to protect a human subject against infection with a plurality of different human coronaviruses. The polypeptide and/or epitope may be conserved between a human coronavirus (such as SARS-COV-2) and one or more animal coronaviruses. In this case, a pharmaceutical composition (e.g. vaccine) comprising the immunogenic peptide of the invention may be capable of preventing the animal coronavirus from crossing the species divide.

### Coronaviruses

The ORF is encoded by at least part of the genome of a coronavirus in the opposite sense to positive sense RNA capable of translation. As set out above, coronaviruses are positive sense ssRNA viruses.

The ORF may, for example, be encoded by all of the genome of a coronavirus in the opposite sense to positive sense RNA capable of translation. Preferably, the ORF is encoded by part of the genome of a coronavirus in the opposite sense to positive sense RNA capable of translation. For instance, the ORF may be encoded by about 1% to about 99%, such as about 2% to about 98%, about 3% to about 97%, about 5% to about 95%, about 10% to about 90%, about 15% to about 85%, about 20% to about 80%, about 25% to about 75%, about 30% to about 70%, about 40% to about 60%, or about 50% of the genome of a coronavirus in the opposite sense to positive sense RNA capable of translation. The ORF may be of any length, as described above.

The coronavirus may, for instance, be a human coronavirus. The coronavirus may, for example, be an animal coronavirus, such as a bat coronavirus. The coronavirus may, for example, be a zoonotic coronavirus. Exemplary human, animal and zoonotic coronaviruses are well-known in the art.

The coronavirus may, for example, be a member of the genus Betacoronavirus. The coronavirus may, for example, be a member of subgenus Sarbecoronavirus.

The coronavirus may, for example, be a SARS-COV-2 virus. The coronavirus may, for example, be a SARS-COV-1 virus. The coronavirus may, for example, be a MERS-COV virus. The coronavirus may, for example, be an endemic common cold coronavirus. Endemic common cold coronaviruses include 229E, NL63, OC43, and HKU1. The coronavirus may, for example, be endemic common cold coronavirus 229E.

The coronavirus may, for example, be a pandemic coronavirus. A pandemic coronavirus may be defined as a coronavirus that has caused, or has the potential to cause, a pandemic. The coronavirus may, for example, be an endemic coronavirus. An endemic coronavirus may be defined as a coronavirus whose infection is constantly maintained at a baseline level in a given geographic area without external inputs. Endemic coronaviruses are well-known in the art, and include common cold coronaviruses. The coronavirus may, for example, be a seasonal coronavirus. A seasonal coronavirus is a coronavirus having a pattern of infection that shows seasonality, that is one or more regular annual peaks.

The positive sense genome of certain coronaviruses, such as SARS-COV-2 and 229E, comprise a gene “orflab”. In SARS-COV-2, the orflab gene corresponds to NCBI gene ID 43740578. When translated in the normal way (i.e. without transcription to form complementary mRNA), the orflab gene contains overlapping open reading frames that encode polyproteins PP1ab and PP1a. The polyproteins are cleaved to yield 16 non-structural proteins, NSP1-16. The inventors have discovered that the orflab gene also encodes, in the opposite sense to positive sense RNA capable of translation, an ORF encoding the polypeptide that gives rise to the CD8+ T cell epitope comprised in the immunogenic peptide of the invention. In other words, the inventors have discovered that the negative sense (i.e. reverse complement of) of the orflab gene also encodes an ORF encoding the polypeptide that gives rise to the CD8+ T cell epitope comprised in the immunogenic peptide of the invention. In other words, at least part of the orflab gene may be transcribed to give complementary mRNA that comprises the ORF encoding the polypeptide giving rise to the epitope comprised in the immunogenic peptide of the invention. The ORF encoding the polypeptide giving rise to the epitope comprised in the immunogenic peptide of the invention may therefore be encoded by at least part of the orflab gene in the opposite sense to positive sense RNA capable of translation.

The ORF may, for example, be encoded by all of the orflab gene of a coronavirus in the opposite sense to positive sense RNA capable of translation. Preferably, the ORF is encoded by part of the orflab gene of a coronavirus in the opposite sense to positive sense RNA capable of translation. For instance, the ORF may be encoded by about 1% to about 99%, such as about 2% to about 98%, about 3% to about 97%, about 5% to about 95%, about 10% to about 90%, about 15% to about 85%, about 20% to about 80%, about 25% to about 75%, about 30% to about 70%, about 40% to about 60%, or about 50% of the orflab gene of a coronavirus in the opposite sense to positive sense RNA capable of translation. The ORF may be of any length, as described above. The orflab gene may, for example, comprise the nucleotide sequence set out in SEQ ID NO: 1. The ORF may, for example, be encoded by nucleotides 1 to 303 of SEQ ID NO: 1.

### Polynucleotide of the Invention

The present invention provides a polynucleotide encoding an immunogenic peptide of the invention. Immunogenic peptides of the invention are described in detail above. Any of the aspects described above in connection with the immunogenic peptide of the invention may apply to the polynucleotide of the invention.

The polynucleotide may comprise RNA. The polynucleotide may comprise DNA. The polynucleotide may comprise both RNA and DNA. Preferably, the polynucleotide comprises or consists of RNA. More preferably, the polynucleotide comprises or consists of mRNA.

The polynucleotide may comprise the sequence:

angncnccnacaacnncggnagnnnncacannacacncaagaacgnnnncngnanggnaggannnnccacnacnncnncag agacnggnnnnagancnncgcaggcaagannanccanncccngcgcgnccncngacnncagnacancaaacgaannngangn nncaacnggnnnngngcnccaaagacaacgnanacaccaggnannnggnnnanacgnggcnnnannagnngcanngnnaac angccaaacaanaggnnnangnaacaannnagencennncnnaaaagagggngngnag (SEQ ID NO: 1) or a variant having at least 50% sequence identity thereto. In SEQ ID NO: 1 each n is independently selected from t and u. In one aspect, each n may, for example, bet. That is, all of the ns may be t. In another aspect, each n may, for example, be u. That is, all of the ns may be u. In a further aspect, some of the ns may be t and some of the ns may be u. That is, the sequence may contain a combination of t and u. The variant may, for example, have at least 55%, at least 60%, at least 65%, at least 70%, at least 75%, at least 80%, at least 85%, at least 90%, at least 95%, at least 97%, at least 98%, or 99% sequence identity to SEQ ID NO: 1.

The polynucleotide may comprise part of the sequence:

angncnccnacaacnneggnagnnnncacannacacncaagaacgnennnengnanggnaggannnnccacnacnncnncag agacnggnnnnagancnncgcaggcaagannanccanncccngcgcgnccncngacnncagnacancaaacgaannngangn nncaacnggnnnngngcnccaaagacaacgnanacaccaggnannnggnnnanacgnggcnnnannagnngcanngnnaac angccaaacaanaggnnnangnaacaannnagencennncnnaaaagagggngngnag (SEQ ID NO: 1), or a variant having at least 50% sequence identity thereto. In SEQ ID NO: 1 each n is independently selected from t and u. In one aspect, each n may, for example, bet. That is, all of the ns may be t. In another aspect, each n may, for example, be u. That is, all of the ns may be u. In a further aspect, some of the ns may be t and some of the ns may be u. That is, the sequence may contain a combination of t and u. The variant may, for example, have at least 55%, at least 60%, at least 65%, at least 70%, at least 75%, at least 80%, at least 85%, at least 90%, at least 95%, at least 97%, at least 98%, or 99% sequence identity to the part of SEQ ID NO: 1. The part of SEQ ID NO: 1 may, for example, be identical to SEQ ID NO: 1 except that the start codon (ATG) is excluded. The part of SEQ ID NO: 1 may, for example, be identical to SEQ ID NO: 1 except that the stop codon (TAG) is excluded. The part of SEQ ID NO: 1 may, for example, be identical to SEQ ID NO: 1 except that the start codon (ATG) and the stop codon (TAG) are excluded. The part of SEQ ID NO: 1 may, for example, encode one or more (such as two or more, three or more, four or more, five or more, six or more, seven or more, eight or more, nine or more, ten or more, or eleven) of SEQ ID NOs: 2 to 12 or a variant thereof having at least 50% sequence identity to the relevant sequence selected from SEQ ID NOs: 2 to 12, in any combination.

### Pharmaceutical Composition

The present invention provides a pharmaceutical composition comprising the immunogenic peptide of the invention or the polynucleotide of the invention. Immunogenic peptides of the invention and polynucleotides of the invention are described in detail above. Any of the aspects described above in connection with the immunogenic peptide of the invention or the polynucleotide of the invention may apply to the polynucleotide of the invention.

The pharmaceutical composition may, for example, be a prophylactic composition, such as a vaccine. The pharmaceutical composition may, for example, be a therapeutic composition. The pharmaceutical composition may have prophylactic effect, therapeutic effect, or both prophylactic and therapeutic effect. In other words, the pharmaceutical composition may have utility in preventing coronavirus infection, treating coronavirus infection, or both preventing and treating coronavirus infection.

### Peptide Compositions

The pharmaceutical composition may comprise one or more immunogenic peptides, such as about one to about 50, about 2 to about 40, about 3 to about 30, about 4 to about 25, about 5 to about 20, about 6 to about 15, about 7, about 8, about 9 or about 10 immunogenic peptides. Each of the one or more immunogenic peptides may, for example, be an immunogenic peptide of the invention. Alternatively, the pharmaceutical composition may contain a mixture of (i) immunogenic peptides of the invention and (ii) other immunogenic peptides.

In one aspect, the pharmaceutical composition comprises two or more immunogenic peptides. Each of the two or more immunogenic peptides may be an immunogenic peptide of the invention. The pharmaceutical composition may, for example, comprise two or more immunogenic peptides each comprising a different immunogenic peptide of the invention. In other words, the pharmaceutical composition may comprise two or more immunogenic peptides that each comprise a different fragment of a polypeptide encoded by an ORF encoded by at least part of the genome of a coronavirus in the opposite sense positive sense RNA capable of translation. The pharmaceutical composition may comprise about one to about 50, about 2 to about 40, about 3 to about 30, about 4 to about 25, about 5 to about 20, about 6 to about 15, about 7, about 8, about 9 or about 10 immunogenic peptides each comprising a different epitope (such as a CD8+ T cell epitope) from a polypeptide encoded by an ORF encoded by at least part of the genome of a coronavirus in the opposite sense to positive sense RNA capable of translation. In some aspects, each of the immunogenic peptides may interact with a different HLA subtype.

The pharmaceutical composition may comprise (i) one or more (such as about 2 to about 40, about 3 to about 30, about 4 to about 25, about 5 to about 20, about 6 to about 15, about 7, about 8, about 9 or about 10) immunogenic peptides that each comprise an epitope from a polypeptide encoded by an ORF encoded by at least part of the genome of a coronavirus in the opposite sense to positive sense RNA capable of translation, and (ii) one or more other immunogenic peptides (i.e. immunogenic peptides that do not comprise an epitope from a polypeptide encoded by an ORF encoded by at least part of the genome of a coronavirus in the opposite sense to positive sense RNA capable of translation). The other immunogenic peptide(s) may comprise one or more epitopes, such as about 2 to 40, about 3 to about 30, about 4 to about 25, about 5 to about 20, about 6 to about 15, about 7, about 8, about 9 or about 10 epitopes. Each of the one or more epitopes may, for example, be a B cell epitope, a CD4+T cell epitope and/or CD8+ T cell epitope. The CD4+T cell epitope may, for example, be a peptide that is expressed by one or more coronaviruses and that is capable of (i) presentation by a class II MHC molecule and (ii) recognition by a T cell receptor (TCR) present on a CD4+T cell. Alternatively, the CD4+T cell epitope may be an CD4+T cell epitope that is not expressed by one or more coronaviruses. The CD8+ T cell epitope may, for example, be a peptide that is expressed by one or more coronaviruses and that is capable of (i) presentation by a class I MHC molecule and (ii) recognition by a T cell receptor (TCR) present on a CD8+T cell. Preferably, the CD8+T cell epitope is a CD8+ T cell epitope that is not a CD8+ T cell epitope from a polypeptide encoded by an ORF encoded by at least part of the genome of a coronavirus in the opposite sense to positive sense RNA capable of translation. The CD8+ T cell epitope may be an CD8+T cell epitope that is not expressed by one or more coronaviruses. The B cell epitope may, for example, be a peptide that is expressed by one or more coronaviruses and that is capable of recognition by a B cell receptor (BCR) present on a B cell. Preferably, the B cell epitope is a B cell epitope that is not a B cell epitope from a polypeptide encoded by an ORF encoded by at least part of the genome of a coronavirus in the opposite sense to positive sense RNA capable of translation. The B cell epitope may be a B cell epitope that is not expressed by one or more coronaviruses.

Many B cell epitopes, CD4+T cell epitopes and CD8+ T cell epitopes (such as B cell epitopes, CD4+T cell epitopes and CD8+ T cell epitopes from coronaviruses) are known in the art. Methods for identifying B cell epitopes, CD4+T cell epitopes and CD8+ T cell epitopes are known in the art. Epitope mapping methods include X-ray co-crystallography, array-based oligo-peptide scanning (sometimes called overlapping peptide scan or pepscan analysis), site-directed mutagenesis, high throughput mutagenesis mapping, hydrogen-deuterium exchange, crosslinking coupled mass spectrometry, phage display and limited proteolysis. MHC motif prediction methodologies may also be used. CD8+ T cell epitopes presented by coronavirus-infected cells can be identified in order to directly identify CD8+ T cell epitopes for inclusion in the pharmaceutical composition, as described above. B cell epitopes may be identified using epitope mapping methods. These methods include structural approaches, wherein the known or modelled structure of a protein is used in an algorithm based approach to predict surface epitopes, and functional approaches, wherein the binding of whole proteins, protein fragments or peptides to an antibody can be quantitated e.g. using an Enzyme-Linked Immunosorbent Assay (ELISA). Competition mapping, antigen modification or protein fragmentation methods may also be used.

The pharmaceutical composition may comprise at least one immunogenic peptide that interacts with at least two different HLA supertypes. This may allow the pharmaceutical composition to elicit an immune response in a greater proportion of individuals to which the pharmaceutical composition is administered. This is because the pharmaceutical composition should be capable of eliciting an immune response (such as a T cell response, e.g. a CD8+T cell response) in all individuals of an HLA supertype that interacts with the immunogenic peptide. The pharmaceutical composition may, for example, comprise at least one, at least two, at least three, at least four, at least five, at least ten, at least fifteen, or at least twenty immunogenic peptides that each comprise a CD8+ T cell epitope from a polypeptide encoded by an ORF encoded by at least part of the genome of a coronavirus in the opposite sense to positive sense RNA capable of translation and interact with at least two different HLA supertypes. Each immunogenic peptide may interact with at least two, at least three, at least four, at least five, at least six, at least 7, at least 8, at least 9 or at least 10 different HLA supertypes. Each immunogenic peptide may interact with two or more of A1, A10, All, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, and C9, or any other HLA supertype known in the art, in any combination.

The pharmaceutical composition may comprise at least two immunogenic peptides that each interact with a different HLA supertype. For example, the pharmaceutical composition may comprise two or more immunogenic peptides that (i) each comprise a different epitope from a polypeptide encoded by an ORF encoded by at least part of the genome of a coronavirus in the opposite sense to positive sense RNA capable of translation, and (ii) each interact with a different HLA supertype. Including two or more such immunogenic peptides in the pharmaceutical composition allows the pharmaceutical composition to elicit an immune response in a greater proportion of individuals to which the pharmaceutical composition is administered. This is because the pharmaceutical composition should be capable of eliciting an immune response (such as a T cell response) in all individuals of an HLA supertype that interacts with one of the epitopes comprised in the two or more immunogenic peptides. Each epitope may interact with A1, A10, All, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, orC9, or any other HLA supertype know in the art. Any combination of immunogenic peptides comprising such an epitope is possible.

### Polynucleotide Composition

The pharmaceutical_composition may comprise one or more polynucleotides, such as about one to about 50, about 2 to about 40, about 3 to about 30, about 4 to about 25, about 5 to about 20, about 6 to about 15, about 7, about 8, about 9 or about 10 polynucleotides. Each of the one or more polynucleotides may, for example, be a polynucleotide of the invention. Alternatively, the pharmaceutical composition may contain a mixture of (i) polynucleotides of the invention and (ii) other polynucleotides.

Each polynucleotide may comprise RNA, such as mRNA. Each polynucleotide may comprise DNA. Each polynucleotide may comprise RNA and DNA. Preferably, each polynucleotide comprises or consists of RNA. More preferably, each polynucleotide comprises or consists of mRNA.

In one aspect, the pharmaceutical composition comprises two or more polynucleotides. Each of the two or more polynucleotides may be a polynucleotide of the invention. The pharmaceutical composition may, for example, comprise two or more polynucleotides each encoding a different immunogenic peptide of the invention. In other words, the pharmaceutical composition may comprise two or more polynucleotides that each encode a different fragment of a polypeptide encoded by an ORF encoded by at least part of the genome of a coronavirus in the opposite sense positive sense RNA capable of translation. The pharmaceutical composition may comprise about one to about 50, about 2 to about 40, about 3 to about 30, about 4 to about 25, about 5 to about 20, about 6 to about 15, about 7, about 8, about 9 or about 10 polynucleotides each encoding a different epitope from a polypeptide encoded by an ORF encoded by at least part of the genome of a coronavirus in the opposite sense to positive sense RNA capable of translation. In some aspects, each of the different epitopes may interact with a different HLA subtype.

The pharmaceutical composition may comprise one or more (such as about 2 to about 40, about 3 to about 30, about 4 to about 25, about 5 to about 20, about 6 to about 15, about 7, about 8, about 9 or about 10) polynucleotides each encoding an immunogenic peptide that comprises an epitope from a polypeptide encoded by an ORF encoded by at least part of the genome of a coronavirus in the opposite sense to positive sense RNA capable of translation, and (ii) one or more other polynucleotides (i.e. polynucleotides that do not encode an immunogenic peptides that comprises an epitope from a polypeptide encoded by an ORF encoded by at least part of the genome of a coronavirus in the opposite sense to positive sense RNA capable of translation). The other polynucleotides may encode one or more epitopes, such as about 2 to 40, about 3 to about 30, about 4 to about 25, about 5 to about 20, about 6 to about 15, about 7, about 8, about 9 or about 10 epitopes. The epitope may, for example, be B cell epitope, a CD4+T cell epitope and/or CD8+ T cell epitope, as described above in connection with peptide vaccines.

The pharmaceutical composition may comprise at least one polynucleotide that encodes an immunogenic peptide that interacts with at least two different HLA supertypes. This allows the pharmaceutical composition to elicit an immune response (such as a T cell response, e.g. a CD8+ T cell response) in a greater proportion of individuals to which the pharmaceutical composition is administered. This is because the pharmaceutical composition should be capable of eliciting an immune response (such as a T cell response, e.g. a CD8+ T cell response) in all individuals of an HLA supertype that interacts with the epitope. The pharmaceutical composition may, for example, comprise at least one, at least two, at least three, at least four, at least five, at least ten, at least fifteen, or at least twenty polynucleotides that each encode an immunogenic peptide that comprises a CD8+ T cell epitope from a polypeptide encoded by an ORF encoded by at least part of the genome of a coronavirus in the opposite sense to positive sense RNA capable of translation and interacts with at least two different HLA supertypes. Each immunogenic peptide may interact with at least two, at least three, at least four, at least five, at least six, at least 7, at least 8, at least 9 or at least 10 different HLA supertypes. Each immunogenic peptide may interact with two or more of A1, A10, All, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, and C9, or any other HLA supertype known in the art, in any combination.

The pharmaceutical composition may comprise at least two polynucleotides that each encode an immunogenic peptide that interacts with a different HLA supertype. For example, the pharmaceutical composition may comprise two or more polynucleotides that each encode an immunogenic peptide that (i) comprises a different epitope from a polypeptide encoded by an ORF encoded by at least part of the genome of a coronavirus in the opposite sense to positive sense RNA capable of translation, and (ii) interacts with a different HLA supertype. Including two or more such polynucleotides in the pharmaceutical composition allows the pharmaceutical composition to elicit an immune response (such as a T cell response, e.g. a CD8+T cell response) in a greater proportion of individuals to which the composition is administered. This is because the pharmaceutical composition should be capable of eliciting an immune response (such as a T cell response, e.g. a CD8+ T cell response) in all individuals of an HLA supertype that interacts with one of the epitopes encoded by the two or more polynucleotides. Each epitope may interact with A1, A10, All, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, or C9, or any other HLA supertype know in the art. Any combination of immunogenic peptides comprising such an epitope is possible.

### Nanoparticles

Any immunogenic peptide or polynucleotide comprised in the pharmaceutical composition of the invention may be attached to a nanoparticle. Attachment to a nanoparticle, for example a gold nanoparticle, is beneficial as described herein.

Attachment of the peptide or polynucleotide to a nanoparticle (such as a gold nanoparticle) reduces or eliminates the need to include a virus or an adjuvant in the pharmaceutical composition. The nanoparticles may contain immune “danger signals” that help to effectively induce an immune response to the peptides or polynucleotides. The nanoparticles may induce dendritic cell (DC) activation and maturation, required for a robust immune response. The nanoparticles may contain non-self components that improve uptake of the nanoparticles and thus the peptides or polynucleotides by cells, such as antigen presenting cells. Attachment of a peptide or polynucleotide to a nanoparticle may therefore enhance the ability of antigen presenting cells to stimulate virus-specific T and/or B cells. Attachment to a nanoparticle also facilitates delivery of the pharmaceutical compositions via the subcutaneous, intradermal, transdermal and oral/buccal routes, providing flexibility in administration.

Nanoparticles are particles between 1 and 100 nanometers (nm) in size which can be used as a substrate for immobilising ligands. In the pharmaceutical compositions of the invention, the nanoparticle may have a mean diameter of 1 to 100, 20 to 90, 30 to 80, 40 to 70 or 50 to 60 nm. Preferably, the nanoparticle has a mean diameter of 20 to 40 nm. A mean diameter of 20 to 40 nm facilitates uptake of the nanoparticle to the cytosol. The mean diameter can be measured using techniques well known in the art such as transmission electron microscopy.

Nanoparticles suitable for the delivery of antigen, such as an immunogenic peptide, are known in the art. Methods for the production of such nanoparticles are also known. Nanoparticles suitable for the delivery of polynucleotides, and methods for their production are also known in the art.

The nanoparticle may, for example, be a polymeric nanoparticle, an inorganic nanoparticle, a liposome, an immune stimulating complex (ISCOM), a virus-like particle (VLP), or a self-assembling protein. The nanoparticle is preferably a calcium phosphate nanoparticle, a silicon nanoparticle or a gold nanoparticle.

The nanoparticle may be a polymeric nanoparticle. The polymeric nanoparticle may comprise one or more synthetic polymers, such as poly (d,1-lactide-co-glycolide) (PLG), poly (d,1-lactic-coglycolic acid) (PLGA), poly(g-glutamic acid) (g-PGA) m poly(ethylene glycol) (PEG), or polystyrene. The polymeric nanoparticle may comprise one or more natural polymers such as a polysaccharide, for example pullulan, alginate, inulin, and chitosan. The use of a polymeric nanoparticle may be advantageous due to the properties of the polymers that may be included in the nanoparticle. For instance, the natural and synthetic polymers recited above may have good biocompatibility and biodegradability, a non-toxic nature and/or the ability to be manipulated into desired shapes and sizes. The polymeric nanoparticle may form a hydrogel nanoparticle. Hydrogel nanoparticles are a type of nano-sized hydrophilic three-dimensional polymer network. Hydrogel nanoparticles have favourable properties including flexible mesh size, large surface area for multivalent conjugation, high water content, and high loading capacity for antigens. Polymers such as Poly(L-lactic acid) (PLA), PLGA, PEG, and polysaccharides are particularly suitable for forming hydrogel nanoparticles.

The nanoparticle may be an inorganic nanoparticle. Typically, inorganic nanoparticles have a rigid structure and are non-biodegradable. However, the inorganic nanoparticle may be biodegradable. The inorganic nanoparticle may comprise a shell in which an antigen may be encapsulated. The inorganic nanoparticle may comprise a core to which an antigen may be covalently attached. The core may comprise a metal. For example, the core may comprise gold (Au), silver (Ag) or copper (Cu) atoms. The core may be formed of more than one type of atom. For instance, the core may comprise an alloy, such as an alloy of Au/Ag, Au/Cu, Au/Ag/Cu, Au/Pt, Au/Pd or Au/Ag/Cu/Pd. The core may comprise calcium phosphate (CaP). The core may comprise a semiconductor material, for example cadmium selenide. The nanoparticle may be a gold nanoparticle, such as a gold glycol-nanoparticle.

Other exemplary inorganic nanoparticles include carbon nanoparticles and silica-based nanoparticles. Carbon nanoparticles have good biocompatibility and can be synthesized into nanotubes and mesoporous spheres. Silica-based nanoparticles (SiNPs) are biocompatible and can be prepared with tunable structural parameters to suit their therapeutic application.

The nanoparticle may be a silicon nanoparticle, such as an elemental silicon nanoparticle. The nanoparticle may be mesoporous or have a honeycomb pore structure. Preferably, the nanoparticle is an elemental silicon particle having a honeycomb pore structure. Such nanoparticles are known in the art and offer tunable and controlled drug loading, targeting and release that can be tailored to almost any load, route of administration, target or release profile. For example, such nanoparticles may increase the bioavailability of their load, and/or improve the intestinal permeability and absorption of orally administered actives. The nanoparticles may have an exceptionally high loading capacity due to their porous structure and large surface area. The nanoparticles may release their load over days, weeks or months, depending on their physical properties. Since silicon is a naturally occurring element of the human body, the nanoparticles may elicit no response from the immune system. This is advantageous to the in vivo safety of the nanoparticles.

Any of the SiNPs described above may be biodegradable or non-biodegradable. A biodegradable SiNP may dissolve to orthosilic acid, the bioavailable form of silicon. Orthosilic acid has been shown to be beneficial for the health of bones, connective tissue, hair, and skin.

The nanoparticle may be a liposome. Liposomes are typically formed from biodegradable, non-toxic phospholipids and comprise a self-assembling phospholipid bilayer shell with an aqueous core. A liposome may be an unilamellar vesicle comprising a single phospholipid bilayer, or a multilameller vesicle that comprises several concentric phospholipid shells separated by layers of water. As a consequence, liposomes can be tailored to incorporate either hydrophilic molecules into the aqueous core or hydrophobic molecules within the phospholipid bilayers. Liposomes may encapsulate antigen within the core for delivery. Liposomes may incorporate viral envelope glycoproteins to the shell to form virosomes. A number of liposome-based products are established in the art and are approved for human use.

The nanoparticle may be an immune-stimulating complex (ISCOM). ISCOMs are cage-like particles which are typically formed from colloidal saponin-containing micelles. ISCOMs may comprise cholesterol, phospholipid (such as phosphatidylethanolamine or phosphatidylcholine) and saponin (such as Quil A from the tree Quillaia saponaria). ISCOMs have traditionally been used to entrap viral envelope proteins, such as envelope proteins from herpes simplex virus type 1, hepatitis B, or influenza virus.

The nanoparticle may be a virus-like particle (VLP). VLPs are self-assembling nanoparticles that lack infectious nucleic acid, which are formed by self-assembly of biocompatible capsid protein. VLPs are typically about 20 to about 150 nm, such as about 20 to about 40 nm, about 30 to about 140 nm, about 40 to about 130 nm, about 50 to about 120 nm, about 60 to about 110 nm, about 70 to about 100 nm, or about 80 to about 90 nm in diameter. VLPs advantageously harness the power of evolved viral structure, which is naturally optimized for interaction with the immune system. The naturally-optimized nanoparticle size and repetitive structural order means that VLPs induce potent immune responses, even in the absence of adjuvant.

The nanoparticle may be a self-assembling protein. For instance, the nanoparticle may comprise ferritin. Ferritin is a protein that can self-assemble into nearly-spherical 10 nm structures. The nanoparticle may comprise major vault protein (MVP). Ninety-six units of MVP can self-assemble into a barrel-shaped vault nanoparticle, with a size of approximately 40 nm wide and 70 nm long.

The nanoparticle may be a calcium phosphate (CaP) nanoparticle. CaP nanoparticles may comprise a core comprising one or more (such as two or more, 10 or more, 20 or more, 50 or more, 100 or more, 200 or more, or 500 or more) molecules of CaP. CaP nanoparticles and methods for their production are known in the art. For instance, a stable nano-suspension of CAP nanoparticles may be generated by mixing inorganic salt solutions of calcium and phosphates in pre-determined ratios under constant mixing.

The CaP nanoparticle may have an average particle size of about 80 to about 100 nm, such as about 82 to about 98 nm, about 84 to about 96 nm, about 86 to about 94 nm, or about 88 to about 92 nm. This particle size may produce a better performance in terms of immune cell uptake and immune response than other, larger particle sizes. The particle size may be stable (i.e. show no significant change), for instance when measured over a period of 1 month, 2 months, 3 months, 6 months, 12 months, 18 months, 24 months, 36 months or 48 months.

CaP nanoparticles can be co-formulated with one or multiple antigens either adsorbed on the surface of the nanoparticle or co-precipitated with CaP during particle synthesis. For example, a peptide, such as an immunogenic peptide, may be attached to the CaP nanoparticle by dissolving the peptide in DMSO (for example at a concentration of about 10 mg/ml), adding to a suspension of CaP nanoparticles together with N-acetyl-glucosamine (GlcNAc) (for example at 0.093 mol/L and ultra-pure water, and mixing at room temperature for a period of about 4 hours (for example, 1 hour, 2 hours, 3 hours, 5 hours, 6 hours, 7 hours, 8 hours, 9 hours or 10 hours).

The pharmaceutical composition may comprise about 0.15 to about 0.8%, such as 0.2 to about 0.75%, 0.25 to about 0.7%, 0.3 to about 0.6%, 0.35 to about 0.65%, 0.4 to about 0.6%, or 0.45 to about 0.55%, CaP nanoparticles. Preferably the pharmaceutical composition comprises about 0.3% CaP nanoparticles.

CaP nanoparticles have a high degree of biocompatibility due to their chemical similarity to human hard tissues such as bone and teeth. Advantageously, therefore, CaP nanoparticles are non-toxic when used for therapeutic applications. CaP nanoparticles are safe for administration via intramuscular, subcutaneous, oral, or inhalation routes. CaP nanoparticles are also simple to synthesise commercially. Furthermore, CaP nanoparticles may be associated with slow release of antigen, which may enhance the induction of an immune response to a peptide attached to the nanoparticle. CaP nanoparticles may be used both as an adjuvant, and as a drug delivery vehicle.

The nanoparticle may be a gold nanoparticle, such as a gold glyconanoparticle. Gold nanoparticles are known in the art and are described in particular in WO 2002/32404, WO 2006/037979, WO 2007/122388, WO 2007/015105 and WO 2013/034726, each of which are incorporated herein by reference in their entirety. The gold nanoparticle attached to each peptide may be a gold nanoparticle described in any of WO 2002/32404, WO 2006/037979, WO 2007/122388, WO 2007/015105 and WO 2013/034726, each of which are incorporated herein by reference in their entirety.

Gold nanoparticles comprise a core comprising a gold (Au) atom. The core may further comprise one or more Fe, Cu or Gd atoms. The core may be formed from a gold alloy, such as Au/Fe, Au/Cu, Au/Gd, Au/Fe/Cu, Au/Fe/Gd or Au/Fe/Cu/Gd. The total number of atoms in the core may be 100 to 500 atoms, such as 150 to 450, 200 to 400 or 250 to 350 atoms. The gold nanoparticle may have a mean diameter of 1 to 100, 20 to 90, 30 to 80, 40 to 70 or 50 to 60 nm. Preferably, the gold nanoparticle has a mean diameter of 20 to 40 nm.

The nanoparticle may comprise a surface coated with alpha-galactose and/or beta-GlcNHAc. For instance, the nanoparticle may comprise a surface passivated with alpha-galactose and/or beta-GlcNHAc. In this case, the nanoparticle may, for example, be a nanoparticle which comprises a core including metal and/or semiconductor atoms. For instance, the nanoparticle may be a gold nanoparticle. Beta-GlcNHAc is a bacterial pathogen-associated-molecular pattern (PAMP), which is capable of activating antigen-presenting cells. In this way, a nanoparticle comprising a surface coated or passivated with Beta-GlcNHAc may non-specifically stimulate an immune response. Attachment of an immunogenic peptide to such a nanoparticle may therefore improve the immune response elicited by administration of the pharmaceutical composition of the invention to an individual.

One or more ligands other than the peptide or polynucleotide may be linked to the nanoparticle, which may be any of the types of nanoparticle described above. The ligands may form a “corona”, a layer or coating which may partially or completely cover the surface of the core. The corona may be considered to be an organic layer that surrounds or partially surrounds the nanoparticle core. The corona may provide or participate in passivating the core of the nanoparticle. Thus, in certain cases the corona may be a sufficiently complete coating layer to stabilise the core. The corona may facilitate solubility, such as water solubility, of the nanoparticles of the present invention.

The nanoparticle may comprise at least 10, at least 20, at least 30, at least 40 or at least 50 ligands. The ligands may include one or more peptides, protein domains, nucleic acid molecules, lipidic groups, carbohydrate groups, anionic groups, or cationic groups, glycolipids and/or glycoproteins. The carbohydrate group may be a polysaccharide, an oligosaccharide or a monosaccharide group (e.g. glucose). One or more of the ligands may be a non-self component, that renders the nanoparticle more likely to be taken up by antigen presenting cells due to its similarity to a pathogenic component. For instance, one or more ligands may comprise a carbohydrate moiety (such as a bacterial carbohydrate moiety), a surfactant moiety and/or a glutathione moiety. Exemplary ligands include glucose, N-acetylglucosamine (GlcNAc), glutathione, 2′-thioethyl-β-D-glucopyranoside and 2′-thioethyl-D-glucopyranoside. Preferred ligands include glycoconjugates, which form glyconanoparticles

Linkage of the ligands to the core may be facilitated by a linker. The linker may comprise a thiol group, an alkyl group, a glycol group or a peptide group. For instance, the linker may comprise C2-C15 alkyl and/or C2-C15 glycol. The linker may comprise a sulphur-containing group, amino-containing group, phosphate-containing group or oxygen-containing group that is capable of covalent attachment to the core. Alternatively, the ligands may be directly linked to the core, for example via a sulphur-containing group, amino-containing group, phosphate-containing group or oxygen-containing group comprised in the ligand.

### Attachment to Nanoparticles

Typically, the immunogenic peptide or polynucleotide may be attached to the core of the nanoparticle, but attachment to the corona or a ligand may also be possible. An immunogenic peptide may be attached at its N-terminus to the nanoparticle.

The peptide or polynucleotide may be directly attached to the nanoparticle, for example by covalent bonding of an atom in a sulphur-containing group, amino-containing group, phosphate-containing group or oxygen-containing group in the peptide or polynucleotide to an atom in the nanoparticle or its core.

A linker may be used to link the peptide or polynucleotide to the nanoparticle. The linker may comprise a sulphur-containing group, amino-containing group, phosphate-containing group or oxygen-containing group that is capable of covalent attachment to an atom in the core. For example, the linker may comprise a thiol group, an alkyl group, a glycol group or a peptide group.

The linker may comprise a peptide portion and a non-peptide portion. The peptide portion may comprise the sequence X₁X₂Z₁, wherein X₁ is an amino acid selected from A and G; X₂ is an amino acid selected from A and G; and Z₁ is an amino acid selected from Y and F. The peptide portion may comprise the sequence AAY or FLAAY. The peptide portion of the linker may be linked to the N-terminus of the peptide. The non-peptide portion of the linker may comprise a C2-C15 alkyl and/a C2-C15 glycol, for example a thioethyl group or a thiopropyl group.

The linker may be (i) HS—(CH₂)₂—CONH-AAY; (ii) HS—(CH₂)₂—CONH-LAAY; (iii) HS—(CH₂)₃—CONH-AAY; (iv) HS—(CH₂)₃—CONH-FLAAY; (v) HS—(CH₂)₁₀—(CH₂OCH₂)₇—CONH-AAY; and (vi) HS—(CH₂)₁₀—(CH₂OCH₂)₇—CONH-FLAAY. In this case, the thiol group of the non-peptide portion of the linker links the linker to the core.

Other suitable linkers for attaching a peptide or polynucleotide to a nanoparticle are known in the art, and may be readily identified and implemented by the skilled person.

As explained above, the pharmaceutical composition may comprise multiple immunogenic peptides or multiple polynucleotides. When the pharmaceutical composition comprises more than one immunogenic peptide or polynucleotide, two or more (such as three or more, four or more, five or more, ten or more, or twenty or more) of the immunogenic peptides or polynucleotides may be attached to the same nanoparticle. Two or more (such as three or more, four or more, five or more, ten or more, or twenty or more) of the immunogenic peptides or polynucleotides may each be attached to different nanoparticle. The nanoparticles to which the immunogenic peptides or polynucleotides are attached may though be the same type of nanoparticle. For instance, each immunogenic peptide or polynucleotide may be attached to a gold nanoparticle or gold glycol-nanoparticle. Each immunogenic peptide or polynucleotide may be attached to a CaP nanoparticle. The nanoparticle to which the immunogenic peptides or polynucleotides are attached may be a different type of nanoparticle. For instance, one immunogenic peptide or polynucleotide may be attached to a gold nanoparticle, and another immunogenic peptide or polynucleotide may be attached to a CaP nanoparticle.

Compositions may be prepared together with a physiologically acceptable carrier or diluent. Typically, such compositions are prepared as liquid suspensions of peptides and/or peptide-linked nanoparticles.

### Medicaments, Methods and Therapeutic Use

The invention provides a method of preventing and/or treating a coronaviral infection, comprising administering the pharmaceutical composition of the invention to an individual. The invention also provides a pharmaceutical composition of the invention for use in a method of preventing and/or treating a coronaviral infection in an individual. The invention further provides the use of immunogenic peptides, and nucleotides encoding such peptides, in the manufacture of a medicament for the treatment and/or prevention of a coronaviral infection in an individual.

Typically, the individual is human. The individual may, however, be an animal such as a dog, cat, rabbit, guinea pig, horse, bovine, sheep, goat, bird, bat and so on.

The coronaviral infection may be caused by a zoonotic virus. The coronaviral infection may be a pandemic viral infection or a potentially pandemic viral infection. The coronaviral infection may be an infection caused by an endemic coronavirus. The coronaviral invention may be a seasonal coronaviral infection. The coronaviral infection may be caused by a human coronavirus. The coronaviral infection may be caused by an animal coronavirus, such as a bat coronavirus. The coronaviral infection may be caused by a zoonotic coronavirus. Exemplary human, animal and zoonotic coronaviruses are well-known in the art.

The human coronavirus may, for example, be a SARS-COV-2 virus. The human coronavirus may, for example, be a SARS-COV-1 virus. The human coronavirus may, for example, be a MERS-COV virus. The human coronavirus may, for example, be an endemic common cold coronavirus. As set out above, endemic common cold coronaviruses include 229E, NL63, OC43, and HKU1.

The pharmaceutical composition preferably comprises a pharmaceutically acceptable carrier or diluent. The pharmaceutical composition may be formulated using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA, which is incorporated herein by reference in its entirety.

The pharmaceutical composition may be administered by any route. Suitable routes include, but are not limited to, the intravenous, intramuscular, intraperitoneal, subcutaneous, intradermal, transdermal and oral/buccal routes.

Nanoparticles may be mixed with an excipient which is pharmaceutically acceptable and compatible with the active ingredient. Suitable excipients are, for example, water, saline, dextrose, glycerol, of the like and combinations thereof.

In addition, if desired, the pharmaceutical compositions may contain minor amounts of auxiliary substances such as wetting or emulsifying agents, and/or pH buffering agents.

The peptides, peptide-linked nanoparticles, polynucleotides, or polynucleotide-linked nanoparticles are administered in a manner compatible with the dosage formulation and in such amount will be therapeutically effective. The quantity to be administered depends on the subject to be treated, the disease to be treated, and the capacity of the subject's immune system. Precise amounts of peptides, polynucleotides or nanoparticles required to be administered may depend on the judgement of the practitioner and may be peculiar to each subject.

Any suitable number of peptides, peptide-linked nanoparticles, polynucleotides, or polynucleotide-linked nanoparticles may be administered to a subject. For example, at least, or about, 0.2×10⁶, 0.25×10⁶, 0.5×10⁶, 1.5×10⁶, 4.0×10⁶ or 5.0×10⁶ peptides, peptide-linked nanoparticles, polynucleotides, or polynucleotide-linked nanoparticles per kg of patient may administered. For example, at least, or about, 10⁵, 10⁶, 10⁷, 10⁸, 10⁹ peptides, peptide-linked nanoparticles, polynucleotides, or polynucleotide-linked nanoparticles may be administered. As a guide, the number of peptides, peptide-linked nanoparticles, polynucleotides, or polynucleotide-linked nanoparticles to be administered may be from 10⁵ to 10⁹, preferably from 10⁶ to 10⁸

### Complexes

The invention provides complex comprising an immunogenic peptide of the invention bound to an MHC molecule. The complex may therefore comprise, for example, an immunogenic peptide comprising or consisting of any one of SEQ ID NOs: 2 to 12, 46 and 63 bound to an MHC molecule. The complex may, for example, therefore comprise an immunogenic peptide having at least 50% sequence identity to the relevant sequence selected from SEQ ID NOs: 2 to 12, 46 and 63. The variant may, for example, have at least 55%, at least 60%, at least 65%, at least 70%, at least 75%, at least 80%, at least 85%, at least 90%, at least 95%, at least 97%, at least 98%, or 99% sequence identity to the relevant sequence selected from SEQ ID NOs: 2 to 12, 46 and 63.

Peptide: MHC binding is well-known in the art. Preferably, the binding between the peptide(s) and MHC molecule(s) comprised in the complex is non-covalent. The binding may be mediated by, for example, electrostatic interaction, hydrogen bonds, van der Waals forces and/or hydrophobic interactions.

The MHC molecule may be a MHC class I molecule or a MHC class II molecule. Preferably, the MHC molecule is an MHC class I molecule. The MHC molecule may be of any HLA supertype. For example, the MHC class I molecule may be of supertype A1, A10, All, A19, A2, A203, A210, A23, A24, A2403, A25, A26, A28, A29, A3, A30, A31, A32, A33, A34, A36, A43, A66, A68, A69, A74, A80, A9, B12, B13, B14, B15, B16, B17, B18, B21, B22, B27, B35, B37, B38, B39, B40, B41, B42, B44, B45, B46, B47, B48, B49, B5, B50, B51, B5102, B5103, B52, B53, B54, B55, B56, B57, B58, B59, B60, B61, B62, B63, B64, B65, B67, B7, B70, B703, B71, B72, B73, B75, B76, B77, B78, B8, B81, B82, C1, C10, C2, C3, C4, C5, C6, C7, C8, or C9.

The complex may comprise two or more immunogenic peptides of the invention, and two or more MHC molecules. For example, the complex may comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more immunogenic peptides of the invention. The complex may comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules. The complex may, for example, comprise three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more immunogenic peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules respectively. The complex may comprise the same number of immunogenic peptides of the invention as MHC molecules. The complex may comprise a different number of immunogenic peptides of the invention from the number MHC molecules. The complex may, for example, comprise four MHC molecules. The complex may comprise or consist of an MHC tetramer. The complex may, for example, comprise twelve MHC molecules. The complex may comprise or consist of an MHC dodecamer.

When the complex comprises two or more immunogenic peptides of the invention, each of the two or more immunogenic peptides may be the same. Alternatively, each of the two or more immunogenic peptides may be different. When the complex comprises three or more immunogenic peptides of the invention, each of the three or more immunogenic peptides may be the same. When the complex comprises three or more immunogenic peptides of the invention, each of the three or more peptides may be different. When the complex comprises three or more immunogenic peptides of the invention, some of the three or more immunogenic peptides may be the same and some of the three of more immunogenic peptides may be different.

When the complex comprises two or more MHC molecules, each of the two or more MHC molecules may be the same. Alternatively, each of the two or more MHC molecules may be different. When the complex comprises three or more MHC molecules, each of the three or more MHC molecules may be the same. When the complex comprises three or more MHC molecules, each of the three or more MHC molecules may be different. When the complex comprises three or more MHC molecules, some of the three or more MHC molecules may be the same and some of the three of more MHC molecules may be different.

When the complex comprises two or more immunogenic peptides of the invention and two or more MHC molecules, each immunogenic peptide may be bound to one of the two or more MHC molecules. That is, each immunogenic peptide comprised in the complex may be bound to an MHC molecule comprised in the complex. Preferably, each immunogenic peptide comprised in the complex is bound to a different MHC molecule comprised in the complex. That is, each MHC molecule comprised in the complex is preferably bound to no more than one immunogenic peptide comprised in the complex. The complex may, however, comprise one or more immunogenic peptides of the invention that are not bound to an MHC molecule. The complex may comprise one or more MHC molecules that are not bound to a peptide of the invention.

The MHC molecule or molecules comprised in the complex may be linked to one another. For example, each of the one or more MHC molecules in the complex may be attached to a backbone molecule or a nanoparticle. The MHC molecule or molecules comprised in the complex may be attached to a dextran backbone. That is, the complex may comprise or consist of an MHC dextramer. Mechanisms for attaching an MHC molecule or molecules to a dextran backbone are known in the art. Any number of MHC molecules may be attached to the dextran backbone. For example, one or more, two or more, three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more MHC molecules may be attached to the dextran backbone.

The complex may comprise a fluorophore. Fluorophores are well-known in the art and include FITC (fluorescein isothiocyanate), PE (phycoerythrin) and APC (allophycocyanin). The complex may comprise any number of fluorophores. For example, the complex may comprise two or more, three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more peptides of the invention and three or more, such as four or more, five or more, six or more, seven or more, eight or more, nine or more, ten or more, 11 or more, 12 or more, 13 or more, 14 or more, 15 or more, 16 or more, 17 or more, 18 or more, 19 or more, or 20 or more fluorophores. When the complex comprise multiple fluorophores, the fluorophores comprised in the complex may be the same or different. When the complex comprises a backbone, such as a dextran backbone, the fluorophore is preferably attached to the dextran backbone. Mechanisms for attaching a fluorophore to a dextran backbone are known in the art.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

### Example 1—Sample Preparation

A three step protocol was used to prepare samples:
1. Generation of virus stocks
2. Generation of infected cell supernatant and lysate samples
3. Inactivation validation of collected samples

### Step 1: Generation of Virus Stocks

SARS COV-2 strains (Washington and Italian) were acquired from the American Type Culture Collection. Vero cells were seeded into a T-75 flask (4*10\hat{ }6 cells/flask). Virus was diluted in 2 mL culture medium to achieve an MOI of 0.02-0.05. Cells were infected for 1 h at 37° C., with rocking every 15 min to spread the inoculum. After 1 h, 8 mL culture medium was added to the flask and flask was returned to the incubator for 3 days at 37° C. and 5% CO₂. At 3 days post infection, 5 mL culture medium was discarded from the flask and 5 mL fresh medium was added. At 5 days post infection, the supernatant was collected (˜10 mL) and syringe filtered (0.2 micron PES filter syringe) into a 50 mL conical flask. Filtered supernatant was aliquoted and frozen at −80° C. Virus was titered using a standard plaque assay with Vero cells.

### Step 2: Generation of Infected Cell Supernatant and Lysate Samples

Samples were collected for the isolation of MHC associated peptides as follows. A new virus stock was generated for each experiment in order to infect sufficient number of cells for sample collection. HepG2 cells were grown in T-175 flasks or T-150 dishes (1*10\hat{ }7 cells/flask) and infected with SARS COV-2. Virus was diluted in 5 ml culture medium to achieve an MOI of 0.01. Cells were infected for 1 h at 37° C., with rocking every 15 min to spread the inoculum. After 1 h, 15 mL culture medium was added to the flask (total 20 mL medium) and flask was returned to the incubator for 3 days at 37° C. and 5% CO₂.

At 72 h, supernatants were diluted 1:1 in fresh culture medium (total volume 20 mL and used to infect HepG2 cells grown in T-150 dishes (1*10\hat{ }7 cells/dish). At 24 h post infection, infected cells were harvested in PBS supplemented with 2 mM EDTA using a cell scraper. Cells were spun down at 1500 rpm, supernatant was removed and the cell pellet was resuspended in a 0.1% Trifluoroacetic acid solution (3 mL per sample). Samples were incubated on a rocker at room temperature for 10 min, and spun down at 1500 rpm for 5 min. The TFA supernatant was collected and stored at −80° C.

The cell pellet was resuspended and washed twice with PBS supplemented with a protease inhibitor cocktail (3 mL per sample). Cells were pelleted at 1500 rpm for 5 min. Cell pellets were resuspended in lysis buffer (150 mM NaCl, 10 mM Na₂HPO₄, 1 mM EDTA, 1% NP-40 and protease inhibitor cocktail, 3 mL per sample). Resuspended cells were frozen at −80° C. and subsequently thawed in a 37° C. water bath twice to lyse cells. Samples were spun down at 3000 rpm for 10 min to discard debris, and cell lysates were frozen at −80° C.

### Step 3: Inactivation Validation of Collected Samples

Prior to removal of collected samples from the BSL-3 laboratory, each sample was individually tested to confirm the absence of detectable virus. Validation was performed using a standard plaque assay with Vero cells (limit of detection ˜50 PFU/mL). Upon confirmation of inactivation, samples were removed from the BSL-3 and shipped.

### Example 2-MHC Class I Peptide Discovery from SARS-COV-2 Virus Infected Cells

Methods

### 1. SCV2 Infected HepG2 Cell Preparation

HepG2 cells (1.4×10⁸ total cells) were grown in two separate batches and both batches were independently infected. For batch 1, cells were infected with SCV2 (MOI=0.01) for 72 h and supernatants were collected. For batch 2, cells were infected with supernatants from round 1 infection (diluted with media 1:1) for 48 h. Infection efficiency was tested for each batch post SCV2 infection using flow cytometry methods. Supernatants and cell lysates were serially diluted 10-fold in Vero cell media (as per plaque assay protocol) and used to infect Vero cells to measure presence of viral particles. Plaque numbers were counted at 48 h post infection as inactivation measure.

### 2. SCV2 MHC Class I Peptide Discovery Using Mass Spectrometry Sample Analysis

a) Sample preparation-SARS-COV-2_USA_WAI strain infected HepG2 cells were harvested and washed by PBS once. The cell pellet was treated with 0.1% TFA and the supernatant was collected. The cell pellet was treated with lysis buffer and processed using the immunoproteomic protocol to isolate and purify MHC class I peptides. The two samples (samples from steps 1&2) were pooled, cleaned up and fractionated on an offline HPLC system. Each fraction was analyzed on a UPLC-Nano-MS/MS system.
b) DDA mode-Samples were analyzed using an Eclipse triploid mass spectrometer coupled to a Dionex Ultimate 3000 RLSCnano System (Thermo Scientific). Samples were loaded on to a fused silica trap column Acclaim PepMap 100, 75 μm×2 cm (ThermoFisher). After washing with 0.1% TFA, the trap column was brought in-line with an analytical column (Nanoease MZ peptide BEH C18, 130A, 1.7 μm, 75 μm×250 mm, Waters) for LC-MS/MS. Peptides were eluted using a segmented linear gradient from 4 to 90% B (A: 0.2% formic acid, B: 0.08% formic acid, 80% ACN): 4-15% B in 5 min, 15-50% B in 50 min, and 50-90% B in 15 min. Mass spectrometry data (CID or HCD mode) was acquired using a data-dependent acquisition procedure with a cyclic series of a full scan from 375-1500 with resolution of 240,000 normalized (AGC) target 250% of normal (1E6), maximum injection time 50 ms. The top S (1 sec) and dynamic exclusion of 30 sec were used for selection of parent ions of charge stage 2-7 for MSMS with intensity threshold at 5E3. The selected ions were transmitted to ion trap with isolation window 1.2 m/z normalized AGC target 200% and fragmented with relative collision energy 35% and scanned in the ion trap with rapid scan rate.

### 3. Database Search

Database searching of all raw spectra files was performed using Proteome Discoverer 1.4 (Thermo Fisher Scientific). SEQUEST was used for database searching against SCV2 protein sequence databases which contain both positive and negative sense sequences. Database searching against the corresponding reverse database was also performed to evaluate the false discovery rate (FDR) of peptide/protein identification. The database searching parameters included up to two missed cleavages for no enzyme digestion, precursor mass tolerance 10 ppm, product ion mass tolerance 0.4 Da, and methionine oxidation as variable modifications. The result from each run was filtered with peptide confidence value as high to obtain FDR less than 5% on peptide level. On the protein level the search parameters included minimum number of peptide 1 for each protein, count only rank 1 peptides, and count peptide only in top scored proteins were applied for all data filtration. In addition, protein grouping was enabled, and strict maximum parsimony principle was applied. Afterwards, manual evaluation was applied into each mass spectra.

### Results

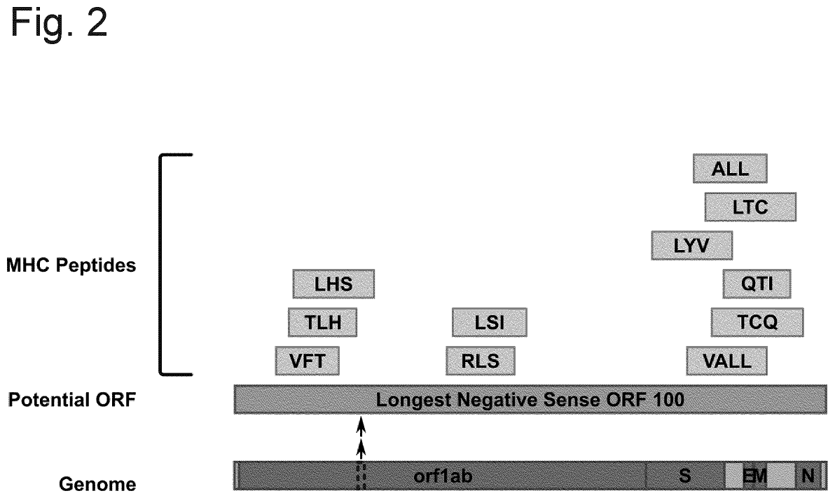

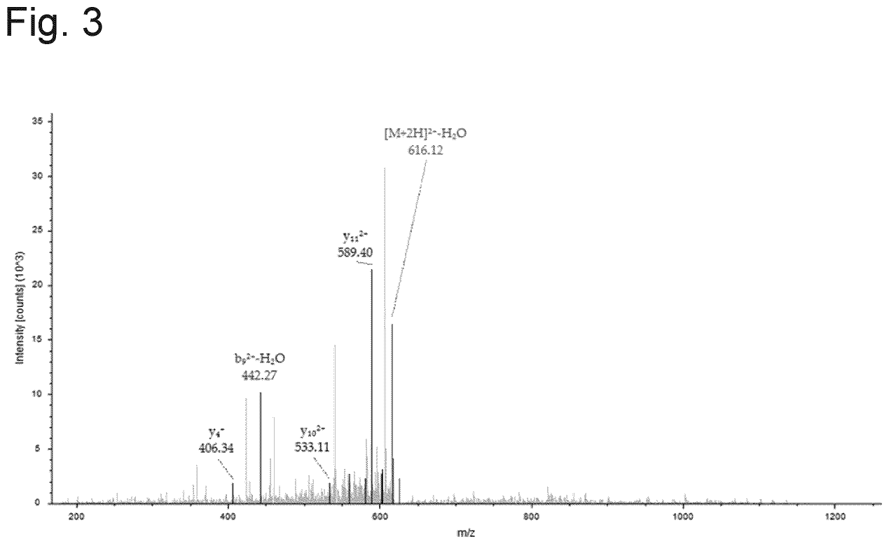

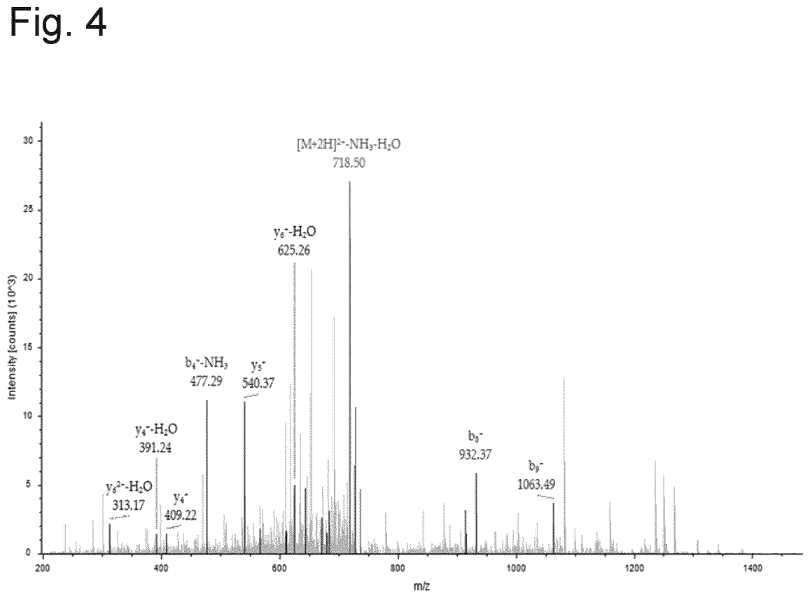

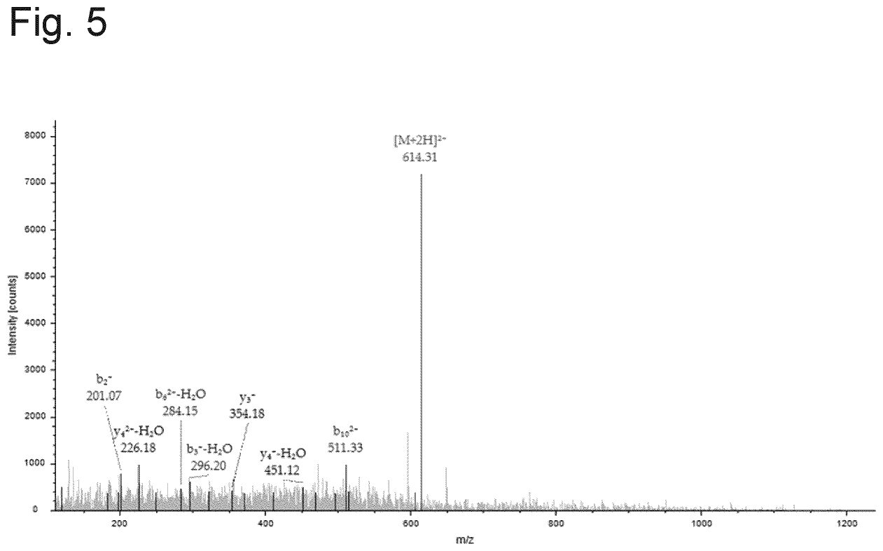

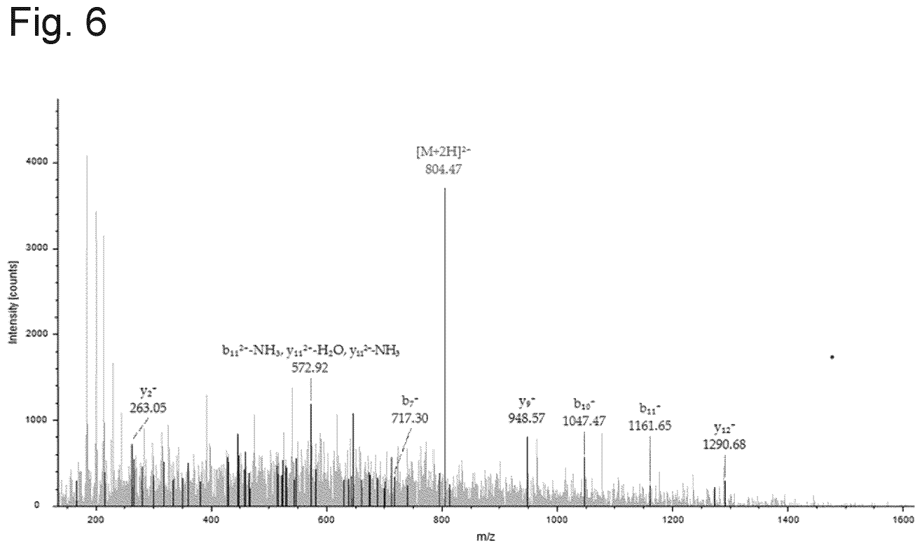

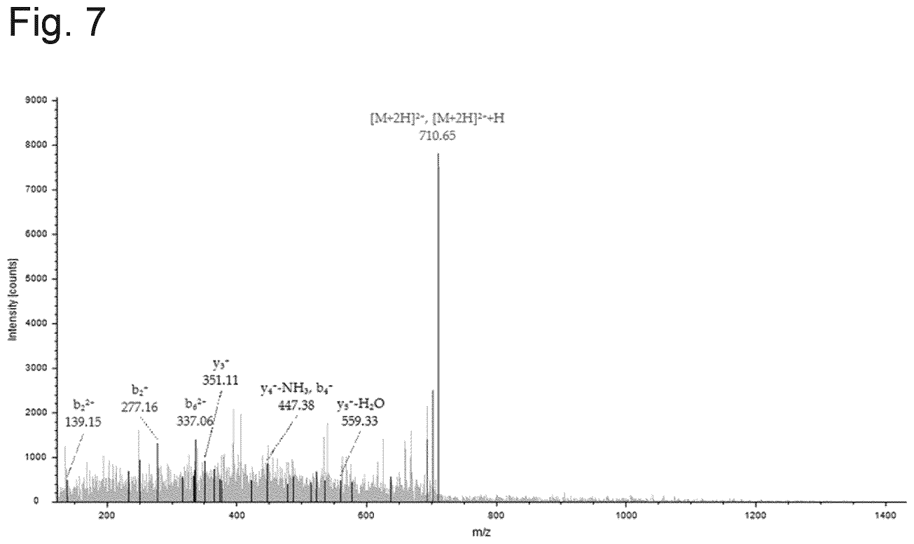

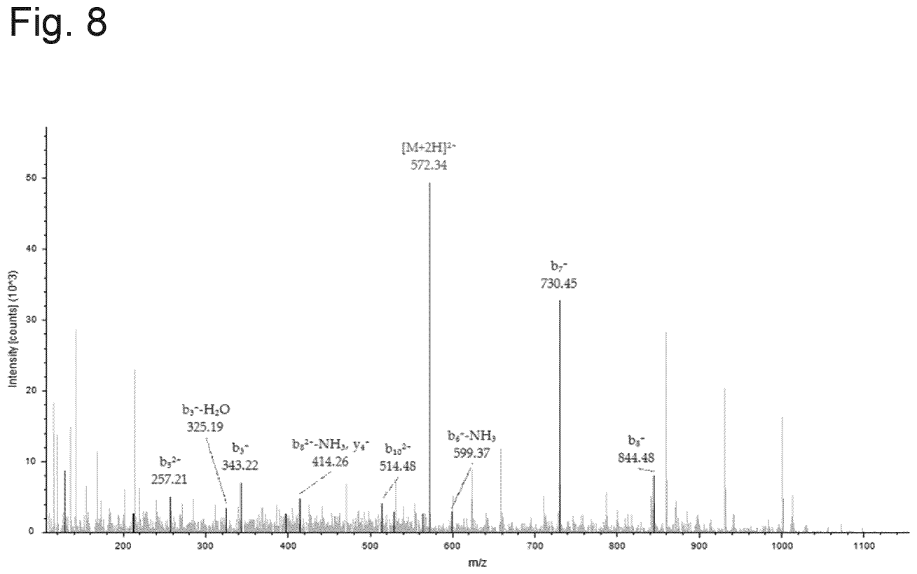

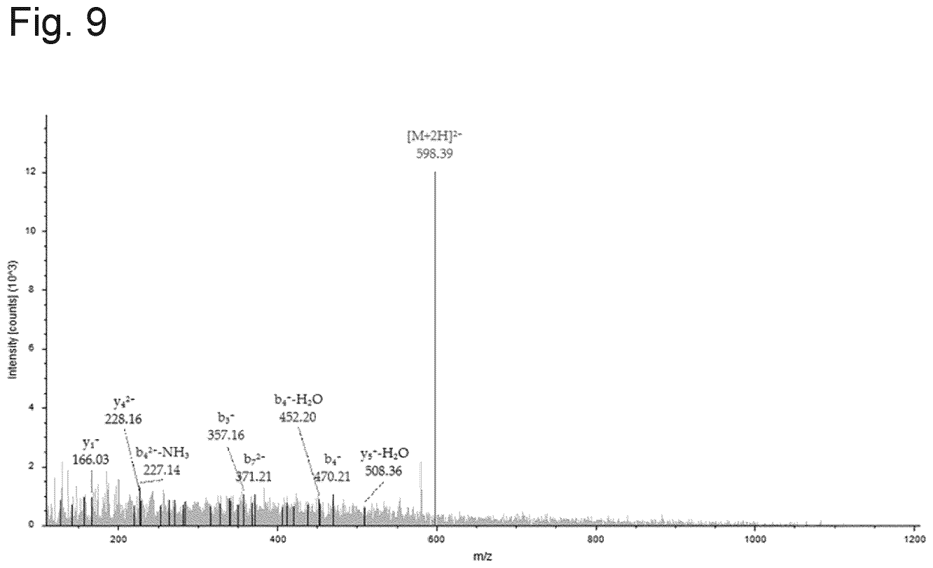

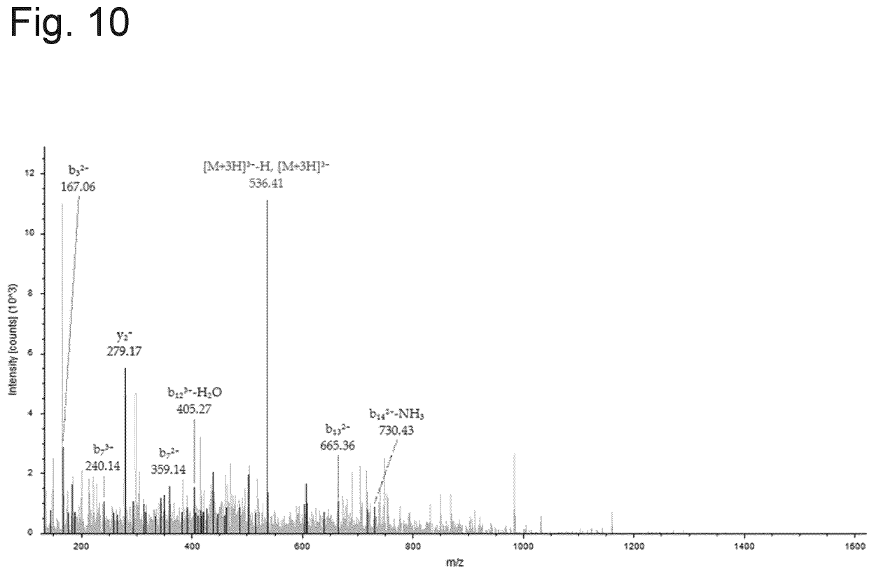

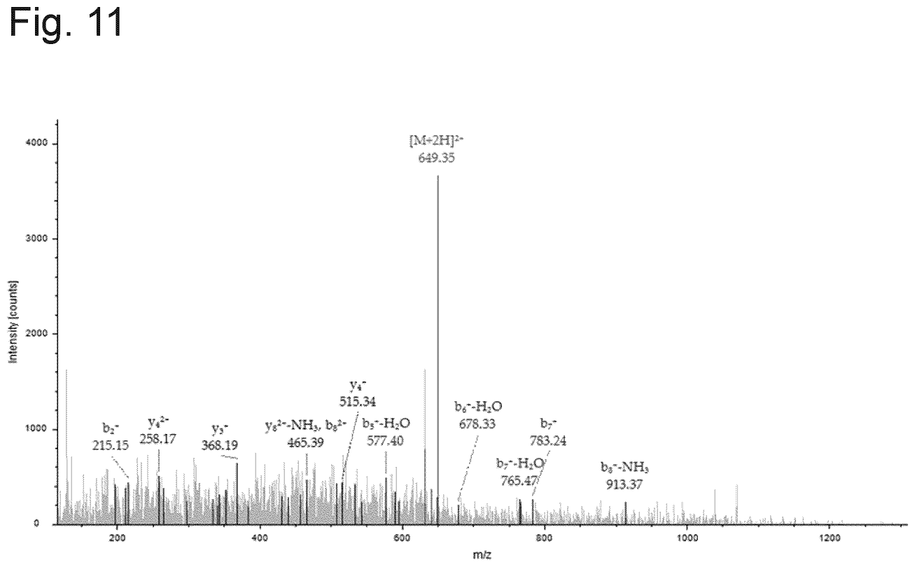

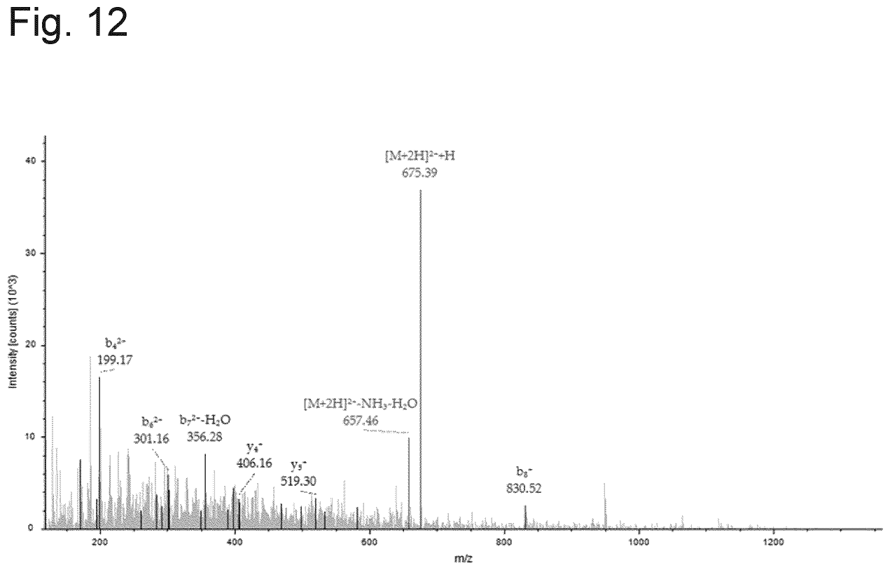

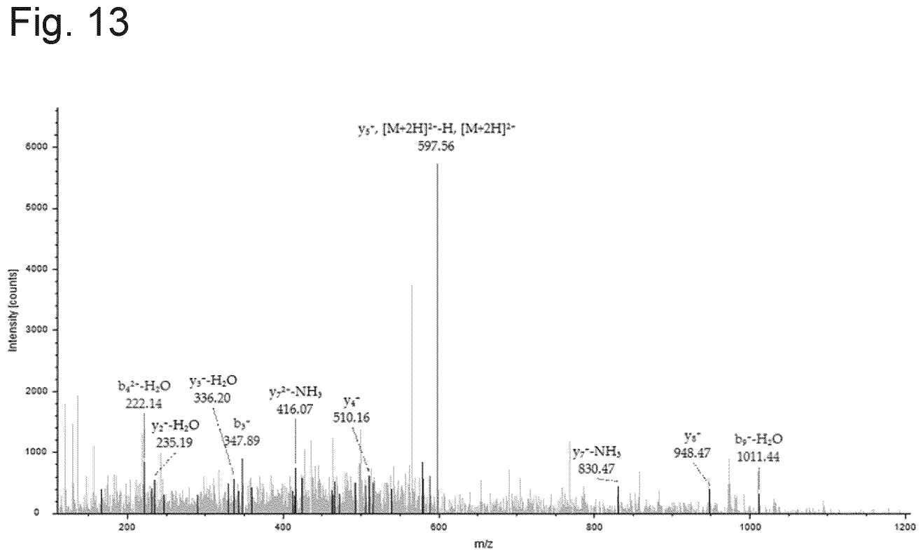

Results are shown in FIGS. 2 to 13 and in Table 2 below.

TABLE 2
List of selected MHC class I peptides from SCV2 viral proteins (negative sense)

| | SEQ | Amino | | | | | | | | | |
|---|---|---|---|---|---|---|---|---|---|---|---|
| | ID | Acid | HLA | Protein Group | | XCorr | XCorr | Charge | m/z [Da] | Charge | m/z [Da] |
| Sequence | NO | length | motif | Accessions | Modifications | CID | HCD | CID | CID | HCD | HCD |
| ALLTCQTIGLCN | 2 | 12 | A2/24 | LNSORF100; | — | 1.87 | — | 2 | 625.31921 | — | — |
| | | | | MN985325_−3_137 | | | | | | | |
| LHSRTSFCMVGFS | 3 | 13 | — | LNSORF100 | — | 1.86 | — | 2 | 736.34497 | — | — |
| LSIPCASSDFST | 4 | 12 | — | LNSORF100; | — | | 2.76 | — | — | 2 | 614.27802 |
| | | | | MN985325_−3_137 | | | | | | | |
| LTCQTIGLCNNLAPF | 5 | 15 | — | LNSORF100; | — | | 3.19 | — | — | 2 | 804.40436 |
| | | | | MN985325_−3_137 | | | | | | | |
| LYVALLVALLTCQ | 6 | 13 | A24, | LNSORF100; | — | 1.91 | 2.42 | 2 | 710.41809 | 2 | 710.41754 |
| | | | C4 | MN985325_−3_137 | | | | | | | |
| QTIGLCNNLAP | 7 | 11 | A2 | LNSORF100; | — | 2.06 | 2.16 | 2 | 572.29523 | 2 | 572.29126 |
| | | | | MN985325_−3_137 | | | | | | | |
| RLSIPCASSDF | 8 | 11 | — | LNSORF100; | — | — | 3.10 | — | — | 2 | 598.29547 |
| | | | | MN985325_−3_137 | | | | | | | |
| TCQTIGLCNNLAPFL | 9 | 15 | | LNSORF100; | | | 2.49 | | | 3 | 536.59845 |
| | | | | MN985325_−3_137 | | | | | | | |
| TLHSRTSFCMV | 10 | 11 | A24 | LNSORF100 | M10(Oxidation) | — | 2.31 | — | — | 2 | 649.30481 |
| VALLTCQTIGLCN | 11 | 13 | A2 | LNSORF100; | — | 1.70 | 2.02 | 2 | 674.85815 | 2 | 674.84967 |
| | | | | MN985325_−3_137 | | | | | | | |
| VFTLHSRTSF | 12 | 10 | — | LNSORF100 | — | — | 2.21 | — | — | 2 | 597.81268 |

### Example 3-Determination of a Consensus Sequence for an ORF Encoded by the Orflab Gene of SARS-COV-2 UK Strains in the Opposite Sense to Positive Sense RNA Capable of Translation

Several thousand UK SARS-COV-2 sequences were downloaded. The longest negative sense ORF encoded by the orflab gene (i.e. the longest ORF encoded by the orflab gene of SARS-COV-2 in the opposite sense to positive sense RNA capable of translation) was determined for each sequence. The Normalised Shannon Entropy for each of the positions in LNSORF100 was calculated. A value of 1 means that the relevant position can be occupied by any of the 20 amino acids. A value of 0 means that only 1 amino acid occupies the relevant position. In other words, in the Normalised Shannon Entropy, a smaller number indicates that the amino acid at a given position is more highly conserved.

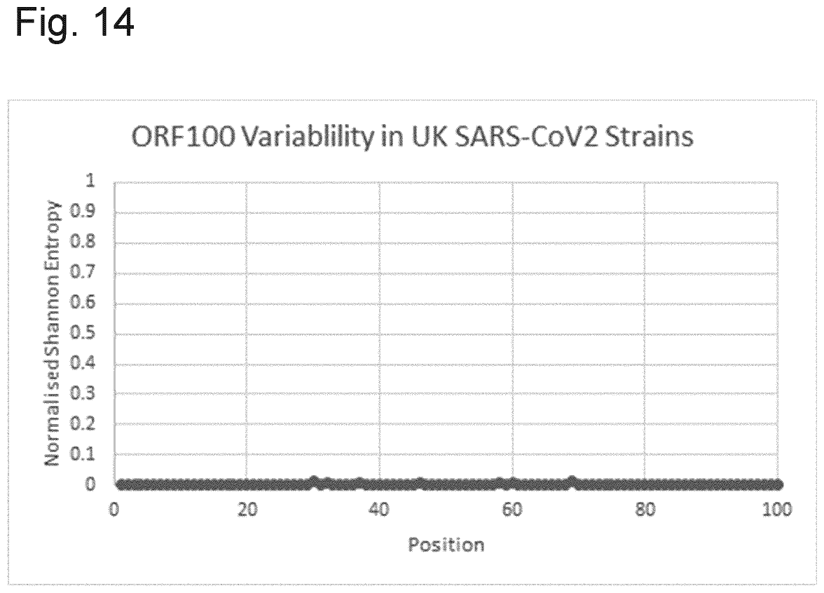

Amino acid frequencies are set out in Table 3 below. Amino acid fractions are shown in Table 4 below. The corresponding Normalised Shannon Entropy and derived consensus sequence (SEQ ID NO: 13) is shown in Table 5 below. As shown in FIG. 14, the ORF is well conserved between UK SARS-COV-2 strains.

TABLE 3
amino acid frequencies

| Pos | A | C | D | E | F | G | H | I | K | L |
|---|---|---|---|---|---|---|---|---|---|---|
| 1 | 0 | 0 | 0 | 0 | 0 | 0 | 0 | 0 | 0 | 0 |
| 2 | 0 | 0 | 0 | 0 | 1 | 0 | 0 | 0 | 0 | 0 |
| 3 | 0 | 0 | 0 | 0 | 0 | 0 | 0 | 0 | 0 | 1 |
| 4 | 0 | 0 | 0 | 0 | 0 | 0 | 0 | 1 | 0 | 0 |
| 5 | 0 | 0 | 0 | 0 | 0 | 0 | 0 | 1 | 0 | 0 |
| 6 | 0 | 0 | 0 | 0 | 0 | 0 | 0 | 0 | 0 | 3 |
| 7 | 0 | 0 | 0 | 0 | 0 | 0 | 0 | 3 | 0 | 0 |
| 8 | 1 | 0 | 0 | 0 | 0 | 73 | 0 | 1 | 0 | 0 |
| 9 | 0 | 2 | 0 | 0 | 135593 | 0 | 0 | 3 | 0 | 64 |
| 10 | 4 | 0 | 0 | 0 | 0 | 0 | 0 | 1 | 67 | 0 |
| 11 | 0 | 0 | 0 | 0 | 0 | 0 | 0 | 0 | 0 | 135665 |
| 12 | 0 | 0 | 0 | 0 | 0 | 0 | 135650 | 0 | 0 | 0 |
| 13 | 1 | 0 | 0 | 0 | 0 | 0 | 0 | 0 | 0 | 0 |
| 14 | 0 | 0 | 0 | 0 | 0 | 0 | 0 | 0 | 39 | 0 |
| 15 | 2 | 0 | 0 | 0 | 0 | 0 | 0 | 0 | 205 | 0 |
| 16 | 0 | 0 | 0 | 0 | 0 | 0 | 0 | 0 | 0 | 0 |
| 17 | 0 | 0 | 0 | 0 | 135659 | 0 | 0 | 1 | 0 | 4 |
| 18 | 0 | 135665 | 0 | 0 | 0 | 0 | 0 | 0 | 0 | 0 |
| 19 | 0 | 0 | 0 | 0 | 0 | 0 | 0 | 5 | 1 | 0 |
| 20 | 5 | 0 | 0 | 0 | 0 | 0 | 0 | 10 | 0 | 0 |
| 21 | 0 | 0 | 0 | 12 | 0 | 135638 | 0 | 0 | 0 | 0 |
| 22 | 0 | 0 | 0 | 0 | 135659 | 0 | 0 | 0 | 0 | 4 |
| 23 | 0 | 0 | 0 | 0 | 6 | 0 | 0 | 0 | 0 | 0 |
| 24 | 1 | 0 | 0 | 0 | 0 | 0 | 0 | 10 | 0 | 0 |
| 25 | 0 | 0 | 0 | 0 | 0 | 0 | 0 | 2 | 0 | 0 |
| 26 | 0 | 0 | 0 | 0 | 5 | 0 | 0 | 0 | 0 | 0 |
| 27 | 0 | 0 | 0 | 0 | 0 | 0 | 0 | 0 | 0 | 0 |
| 28 | 2 | 0 | 2 | 135644 | 0 | 3 | 0 | 0 | 14 | 0 |
| 29 | 1 | 0 | 0 | 0 | 0 | 0 | 0 | 1 | 0 | 0 |
| 30 | 0 | 0 | 341 | 0 | 0 | 135045 | 0 | 0 | 0 | 0 |
| 31 | 0 | 0 | 0 | 0 | 135662 | 0 | 0 | 1 | 0 | 1 |
| 32 | 0 | 0 | 0 | 0 | 0 | 0 | 0 | 1 | 348 | 0 |
| 33 | 5 | 0 | 0 | 0 | 4 | 0 | 0 | 0 | 0 | 0 |
| 34 | 0 | 0 | 0 | 0 | 0 | 0 | 0 | 0 | 0 | 5 |
| 35 | 0 | 0 | 0 | 0 | 0 | 0 | 0 | 0 | 0 | 0 |
| 36 | 135655 | 0 | 0 | 0 | 0 | 0 | 0 | 0 | 0 | 0 |
| 37 | 0 | 0 | 0 | 0 | 0 | 2 | 0 | 1 | 256 | 0 |
| 38 | 0 | 0 | 0 | 0 | 5 | 0 | 0 | 1 | 0 | 135659 |
| 39 | 0 | 0 | 0 | 0 | 0 | 0 | 0 | 0 | 0 | 0 |
| 40 | 0 | 0 | 0 | 0 | 0 | 0 | 0 | 135650 | 0 | 0 |
| 41 | 0 | 0 | 0 | 0 | 0 | 0 | 0 | 0 | 0 | 15 |
| 42 | 0 | 135641 | 0 | 0 | 0 | 0 | 0 | 0 | 0 | 0 |
| 43 | 135649 | 0 | 0 | 1 | 0 | 0 | 0 | 0 | 0 | 0 |
| 44 | 0 | 0 | 0 | 0 | 4 | 0 | 0 | 0 | 0 | 0 |
| 45 | 1 | 0 | 0 | 0 | 1 | 0 | 0 | 0 | 0 | 0 |
| 46 | 0 | 0 | 135403 | 210 | 0 | 0 | 0 | 0 | 0 | 0 |
| 47 | 0 | 0 | 0 | 0 | 135651 | 0 | 0 | 0 | 0 | 7 |
| 48 | 0 | 0 | 0 | 0 | 0 | 0 | 0 | 2 | 0 | 0 |
| 49 | 2 | 0 | 0 | 0 | 0 | 0 | 0 | 3 | 117 | 0 |
| 50 | 0 | 0 | 0 | 0 | 0 | 0 | 0 | 0 | 0 | 25 |
| 51 | 0 | 0 | 0 | 0 | 0 | 0 | 0 | 0 | 50 | 0 |
| 52 | 0 | 0 | 0 | 135565 | 0 | 0 | 0 | 0 | 100 | 0 |
| 53 | 0 | 0 | 0 | 0 | 135661 | 0 | 0 | 0 | 0 | 4 |
| 54 | 0 | 0 | 135647 | 0 | 0 | 0 | 0 | 0 | 0 | 0 |
| 55 | 0 | 0 | 0 | 0 | 0 | 0 | 0 | 7 | 0 | 0 |
| 56 | 0 | 0 | 0 | 0 | 0 | 0 | 0 | 0 | 0 | 1 |
| 57 | 0 | 0 | 0 | 0 | 0 | 0 | 0 | 0 | 0 | 0 |
| 58 | 0 | 3 | 534 | 0 | 0 | 135078 | 0 | 0 | 0 | 0 |
| 59 | 0 | 0 | 0 | 0 | 135660 | 0 | 0 | 0 | 0 | 3 |
| 60 | 4 | 0 | 0 | 0 | 0 | 0 | 0 | 0 | 0 | 195 |
| 61 | 0 | 0 | 0 | 0 | 1 | 0 | 0 | 2 | 0 | 135625 |
| 62 | 0 | 0 | 0 | 0 | 0 | 0 | 0 | 0 | 1 | 2 |
| 63 | 0 | 0 | 0 | 0 | 0 | 0 | 0 | 0 | 0 | 0 |
| 64 | 0 | 0 | 0 | 0 | 0 | 0 | 0 | 0 | 0 | 0 |
| 65 | 0 | 1 | 0 | 0 | 0 | 0 | 1 | 0 | 0 | 0 |
| 66 | 0 | 0 | 0 | 0 | 0 | 0 | 0 | 135648 | 0 | 0 |
| 67 | 0 | 0 | 0 | 0 | 0 | 0 | 135664 | 0 | 0 | 0 |
| 68 | 0 | 0 | 0 | 0 | 0 | 0 | 1 | 0 | 0 | 0 |
| 69 | 1 | 0 | 0 | 0 | 0 | 0 | 0 | 886 | 0 | 7 |
| 70 | 0 | 0 | 0 | 0 | 135665 | 0 | 0 | 0 | 0 | 0 |
| 71 | 0 | 0 | 2 | 0 | 0 | 135663 | 0 | 0 | 0 | 0 |
| 72 | 0 | 0 | 0 | 0 | 0 | 0 | 0 | 0 | 0 | 135663 |
| 73 | 0 | 0 | 0 | 0 | 0 | 0 | 2 | 0 | 0 | 0 |
| 74 | 1 | 0 | 0 | 0 | 0 | 0 | 0 | 0 | 0 | 0 |
| 75 | 135641 | 0 | 0 | 0 | 0 | 0 | 0 | 0 | 0 | 0 |
| 76 | 0 | 0 | 0 | 0 | 0 | 0 | 0 | 0 | 0 | 135665 |
| 77 | 0 | 0 | 0 | 0 | 0 | 0 | 0 | 0 | 0 | 135664 |
| 78 | 0 | 0 | 0 | 0 | 0 | 0 | 0 | 30 | 0 | 0 |
| 79 | 135585 | 0 | 0 | 0 | 0 | 0 | 0 | 0 | 0 | 0 |
| 80 | 0 | 0 | 0 | 0 | 0 | 0 | 0 | 0 | 0 | 135665 |
| 81 | 0 | 0 | 0 | 0 | 0 | 0 | 0 | 0 | 0 | 135652 |
| 82 | 1 | 0 | 0 | 0 | 0 | 0 | 0 | 1 | 0 | 0 |
| 83 | 0 | 135665 | 0 | 0 | 0 | 0 | 0 | 0 | 0 | 0 |
| 84 | 0 | 0 | 0 | 0 | 0 | 0 | 2 | 0 | 0 | 0 |
| 85 | 0 | 0 | 0 | 0 | 0 | 0 | 0 | 1 | 0 | 0 |
| 86 | 0 | 0 | 0 | 0 | 0 | 0 | 0 | 135664 | 0 | 0 |
| 87 | 0 | 0 | 1 | 0 | 0 | 135664 | 0 | 0 | 0 | 0 |
| 88 | 0 | 0 | 0 | 0 | 0 | 0 | 0 | 0 | 0 | 135665 |
| 89 | 0 | 135664 | 0 | 0 | 0 | 0 | 0 | 0 | 0 | 0 |
| 90 | 0 | 0 | 0 | 0 | 0 | 0 | 0 | 0 | 1 | 0 |
| 91 | 0 | 0 | 0 | 0 | 0 | 0 | 0 | 0 | 0 | 0 |
| 92 | 0 | 0 | 0 | 0 | 0 | 0 | 0 | 0 | 0 | 135665 |
| 93 | 135659 | 0 | 1 | 0 | 0 | 0 | 0 | 0 | 0 | 0 |
| 94 | 0 | 0 | 0 | 0 | 0 | 0 | 0 | 0 | 0 | 0 |
| 95 | 0 | 0 | 0 | 0 | 135665 | 0 | 0 | 0 | 0 | 0 |
| 96 | 0 | 0 | 0 | 0 | 0 | 0 | 0 | 0 | 0 | 135665 |
| 97 | 0 | 0 | 0 | 0 | 0 | 0 | 0 | 0 | 135665 | 0 |
| 98 | 1 | 0 | 32 | 135622 | 0 | 1 | 0 | 0 | 9 | 0 |
| 99 | 0 | 0 | 3 | 0 | 0 | 135596 | 0 | 0 | 0 | 0 |
| 100 | 0 | 0 | 0 | 0 | 0 | 0 | 0 | 0 | 0 | 0 |

amino acid frequencies

| Pos | M | N | P | Q | R | S | T | V | W | Y |
|---|---|---|---|---|---|---|---|---|---|---|
| 1 | 135665 | 0 | 0 | 0 | 0 | 0 | 0 | 0 | 0 | 0 |
| 2 | 0 | 0 | 1 | 0 | 0 | 135661 | 0 | 0 | 0 | 2 |
| 3 | 0 | 0 | 135664 | 0 | 0 | 0 | 0 | 0 | 0 | 0 |
| 4 | 0 | 0 | 0 | 0 | 0 | 0 | 135664 | 0 | 0 | 0 |
| 5 | 0 | 55 | 0 | 0 | 0 | 0 | 135609 | 0 | 0 | 0 |
| 6 | 0 | 0 | 1 | 0 | 0 | 135660 | 1 | 0 | 0 | 0 |
| 7 | 0 | 0 | 0 | 0 | 0 | 0 | 0 | 135662 | 0 | 0 |
| 8 | 0 | 0 | 0 | 0 | 0 | 0 | 0 | 135590 | 0 | 0 |
| 9 | 0 | 0 | 0 | 0 | 0 | 3 | 0 | 0 | 0 | 0 |
| 10 | 0 | 0 | 0 | 0 | 1 | 0 | 135592 | 0 | 0 | 0 |
| 11 | 0 | 0 | 0 | 0 | 0 | 0 | 0 | 0 | 0 | 0 |
| 12 | 0 | 0 | 0 | 15 | 0 | 0 | 0 | 0 | 0 | 0 |
| 13 | 0 | 0 | 1 | 0 | 0 | 135663 | 0 | 0 | 0 | 0 |
| 14 | 0 | 0 | 0 | 0 | 135619 | 7 | 0 | 0 | 0 | 0 |
| 15 | 4 | 0 | 0 | 0 | 0 | 0 | 135454 | 0 | 0 | 0 |
| 16 | 0 | 0 | 0 | 0 | 0 | 135664 | 0 | 0 | 0 | 1 |
| 17 | 0 | 0 | 0 | 0 | 0 | 1 | 0 | 0 | 0 | 0 |
| 18 | 0 | 0 | 0 | 0 | 0 | 0 | 0 | 0 | 0 | 0 |
| 19 | 135640 | 0 | 0 | 0 | 0 | 0 | 1 | 18 | 0 | 0 |
| 20 | 0 | 0 | 0 | 0 | 0 | 0 | 0 | 135650 | 0 | 0 |
| 21 | 0 | 0 | 0 | 0 | 15 | 0 | 0 | 0 | 0 | 0 |
| 22 | 0 | 0 | 0 | 0 | 0 | 2 | 0 | 0 | 0 | 0 |
| 23 | 0 | 0 | 3 | 0 | 0 | 135656 | 0 | 0 | 0 | 0 |
| 24 | 0 | 23 | 0 | 0 | 0 | 0 | 135631 | 0 | 0 | 0 |
| 25 | 0 | 4 | 0 | 0 | 0 | 0 | 135659 | 0 | 0 | 0 |
| 26 | 0 | 0 | 1 | 0 | 0 | 135659 | 0 | 0 | 0 | 0 |
| 27 | 0 | 0 | 9 | 0 | 0 | 135656 | 0 | 0 | 0 | 0 |
| 28 | 0 | 0 | 0 | 0 | 0 | 0 | 0 | 0 | 0 | 0 |
| 29 | 0 | 66 | 0 | 0 | 0 | 0 | 135597 | 0 | 0 | 0 |
| 30 | 0 | 0 | 0 | 0 | 0 | 278 | 0 | 1 | 0 | 0 |
| 31 | 0 | 0 | 0 | 0 | 0 | 0 | 0 | 0 | 0 | 1 |
| 32 | 0 | 0 | 0 | 0 | 135316 | 0 | 0 | 0 | 0 | 0 |
| 33 | 0 | 0 | 2 | 0 | 0 | 135632 | 0 | 0 | 0 | 22 |
| 34 | 0 | 0 | 1 | 0 | 0 | 135659 | 0 | 0 | 0 | 0 |
| 35 | 0 | 0 | 0 | 135665 | 0 | 0 | 0 | 0 | 0 | 0 |
| 36 | 0 | 0 | 0 | 0 | 0 | 0 | 9 | 1 | 0 | 0 |
| 37 | 0 | 0 | 0 | 0 | 135406 | 0 | 0 | 0 | 0 | 0 |
| 38 | 0 | 0 | 0 | 0 | 0 | 0 | 0 | 0 | 0 | 0 |
| 39 | 0 | 0 | 17 | 0 | 0 | 135631 | 0 | 0 | 0 | 17 |
| 40 | 2 | 0 | 0 | 0 | 0 | 0 | 13 | 0 | 0 | 0 |
| 41 | 0 | 0 | 135646 | 0 | 0 | 2 | 2 | 0 | 0 | 0 |
| 42 | 0 | 0 | 0 | 0 | 4 | 20 | 0 | 0 | 0 | 0 |
| 43 | 0 | 0 | 0 | 0 | 0 | 0 | 4 | 11 | 0 | 0 |
| 44 | 0 | 0 | 5 | 0 | 0 | 135654 | 0 | 0 | 0 | 2 |
| 45 | 0 | 0 | 0 | 0 | 0 | 135663 | 0 | 0 | 0 | 0 |
| 46 | 0 | 52 | 0 | 0 | 0 | 0 | 0 | 0 | 0 | 0 |
| 47 | 0 | 0 | 0 | 0 | 0 | 6 | 0 | 1 | 0 | 0 |
| 48 | 0 | 20 | 0 | 0 | 0 | 135643 | 0 | 0 | 0 | 0 |
| 49 | 0 | 0 | 0 | 0 | 0 | 0 | 135543 | 0 | 0 | 0 |
| 50 | 0 | 0 | 1 | 0 | 0 | 135639 | 0 | 0 | 0 | 0 |
| 51 | 0 | 135612 | 0 | 0 | 0 | 0 | 0 | 0 | 0 | 3 |
| 52 | 0 | 0 | 0 | 0 | 0 | 0 | 0 | 0 | 0 | 0 |
| 53 | 0 | 0 | 0 | 0 | 0 | 0 | 0 | 0 | 0 | 0 |
| 54 | 0 | 18 | 0 | 0 | 0 | 0 | 0 | 0 | 0 | 0 |
| 55 | 0 | 0 | 0 | 0 | 0 | 0 | 0 | 135658 | 0 | 0 |
| 56 | 0 | 0 | 22 | 0 | 0 | 135642 | 0 | 0 | 0 | 0 |
| 57 | 0 | 16 | 0 | 0 | 0 | 0 | 135649 | 0 | 0 | 0 |
| 58 | 0 | 0 | 0 | 0 | 0 | 49 | 0 | 1 | 0 | 0 |
| 59 | 0 | 0 | 0 | 0 | 0 | 2 | 0 | 0 | 0 | 0 |
| 60 | 108 | 0 | 0 | 0 | 0 | 0 | 0 | 135358 | 0 | 0 |
| 61 | 0 | 0 | 3 | 0 | 34 | 0 | 0 | 0 | 0 | 0 |
| 62 | 0 | 0 | 0 | 135661 | 1 | 0 | 0 | 0 | 0 | 0 |
| 63 | 0 | 0 | 0 | 0 | 135665 | 0 | 0 | 0 | 0 | 0 |
| 64 | 0 | 0 | 0 | 135665 | 0 | 0 | 0 | 0 | 0 | 0 |
| 65 | 0 | 0 | 0 | 0 | 135663 | 0 | 0 | 0 | 0 | 0 |
| 66 | 2 | 0 | 0 | 0 | 0 | 0 | 10 | 5 | 0 | 0 |
| 67 | 0 | 0 | 0 | 0 | 1 | 0 | 0 | 0 | 0 | 0 |
| 68 | 0 | 0 | 0 | 135664 | 0 | 0 | 0 | 0 | 0 | 0 |
| 69 | 0 | 0 | 0 | 0 | 0 | 0 | 0 | 134771 | 0 | 0 |
| 70 | 0 | 0 | 0 | 0 | 0 | 0 | 0 | 0 | 0 | 0 |
| 71 | 0 | 0 | 0 | 0 | 0 | 0 | 0 | 0 | 0 | 0 |
| 72 | 0 | 0 | 0 | 0 | 0 | 2 | 0 | 0 | 0 | 0 |
| 73 | 0 | 0 | 0 | 0 | 0 | 0 | 0 | 0 | 0 | 135663 |
| 74 | 2 | 0 | 0 | 0 | 0 | 0 | 0 | 135662 | 0 | 0 |
| 75 | 0 | 0 | 0 | 0 | 0 | 2 | 18 | 4 | 0 | 10 |
| 76 | 0 | 0 | 0 | 0 | 0 | 0 | 0 | 0 | 0 | 0 |
| 77 | 0 | 0 | 0 | 0 | 0 | 0 | 0 | 1 | 0 | 0 |
| 78 | 0 | 0 | 0 | 0 | 0 | 0 | 0 | 135635 | 0 | 0 |
| 79 | 0 | 0 | 0 | 0 | 0 | 0 | 80 | 0 | 0 | 0 |
| 80 | 0 | 0 | 0 | 0 | 0 | 0 | 0 | 0 | 0 | 0 |
| 81 | 0 | 0 | 0 | 0 | 0 | 13 | 0 | 0 | 0 | 0 |
| 82 | 0 | 0 | 0 | 0 | 0 | 0 | 135663 | 0 | 0 | 0 |
| 83 | 0 | 0 | 0 | 0 | 0 | 0 | 0 | 0 | 0 | 0 |
| 84 | 0 | 0 | 0 | 135663 | 0 | 0 | 0 | 0 | 0 | 0 |
| 85 | 0 | 0 | 0 | 0 | 0 | 0 | 135664 | 0 | 0 | 0 |
| 86 | 1 | 0 | 0 | 0 | 0 | 0 | 0 | 0 | 0 | 0 |
| 87 | 0 | 0 | 0 | 0 | 0 | 0 | 0 | 0 | 0 | 0 |
| 88 | 0 | 0 | 0 | 0 | 0 | 0 | 0 | 0 | 0 | 0 |
| 89 | 0 | 0 | 0 | 0 | 0 | 0 | 0 | 0 | 0 | 1 |
| 90 | 0 | 135664 | 0 | 0 | 0 | 0 | 0 | 0 | 0 | 0 |
| 91 | 0 | 135665 | 0 | 0 | 0 | 0 | 0 | 0 | 0 | 0 |
| 92 | 0 | 0 | 0 | 0 | 0 | 0 | 0 | 0 | 0 | 0 |
| 93 | 0 | 0 | 0 | 0 | 0 | 0 | 5 | 0 | 0 | 0 |
| 94 | 0 | 0 | 135665 | 0 | 0 | 0 | 0 | 0 | 0 | 0 |
| 95 | 0 | 0 | 0 | 0 | 0 | 0 | 0 | 0 | 0 | 0 |
| 96 | 0 | 0 | 0 | 0 | 0 | 0 | 0 | 0 | 0 | 0 |
| 97 | 0 | 0 | 0 | 0 | 0 | 0 | 0 | 0 | 0 | 0 |
| 98 | 0 | 0 | 0 | 0 | 0 | 0 | 0 | 0 | 0 | 0 |
| 99 | 0 | 0 | 0 | 0 | 0 | 29 | 0 | 37 | 0 | 0 |
| 100 | 0 | 0 | 0 | 0 | 0 | 0 | 0 | 135665 | 0 | 0 |

TABLE 4
amino acid fractions

| Pos | A | C | D | E | F | G | H | I | K | L | M | N | P | Q | R | S | T | V | W | Y |
|---|---|---|---|---|---|---|---|---|---|---|---|---|---|---|---|---|---|---|---|---|
| 1 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 |
| 2 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 3 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 |
| 4 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 5 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 6 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 7 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 |
| 8 | 0.000 | 0.000 | 0.000 | 0.000 | 0.000 | 0.001 | 0.000 | 0.000 | 0.000 | 0.000 | 0.000 | 0.000 | 0.000 | 0.000 | 0.000 | 0.000 | 0.000 | 0.999 | 0.000 | 0.000 |
| 9 | 0.000 | 0.000 | 0.000 | 0.000 | 0.999 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 10 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.999 | 0.000 | 0.000 | 0.000 |
| 11 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 12 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 |
| 13 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 14 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 |
| 15 | 0.000 | 0.000 | 0.000 | 0.000 | 0.000 | 0.000 | 0.000 | 0.000 | 0.002 | 0.000 | 0.000 | 0.000 | 0.000 | 0.000 | 0.000 | 0.000 | 0.998 | 0.000 | 0.000 | 0.000 |
| 16 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 17 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 18 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 19 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 |
| 20 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 |
| 21 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 22 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 23 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 24 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 25 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 26 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 27 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 28 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 29 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.999 | 0.000 | 0.000 | 0.000 |
| 30 | 0.000 | 0.000 | 0.003 | 0.000 | 0.000 | 0.995 | 0.000 | 0.000 | 0.000 | 0.000 | 0.000 | 0.000 | 0.000 | 0.000 | 0.000 | 0.002 | 0.000 | 0.000 | 0.000 | 0.000 |
| 31 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 32 | 0.000 | 0.000 | 0.000 | 0.000 | 0.000 | 0.000 | 0.000 | 0.000 | 0.003 | 0.000 | 0.000 | 0.000 | 0.000 | 0.000 | 0.997 | 0.000 | 0.000 | 0.000 | 0.000 | 0.000 |
| 33 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 34 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 35 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 |
| 36 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 37 | 0.000 | 0.000 | 0.000 | 0.000 | 0.000 | 0.000 | 0.000 | 0.000 | 0.002 | 0.000 | 0.000 | 0.000 | 0.000 | 0.000 | 0.998 | 0.000 | 0.000 | 0.000 | 0.000 | 0.000 |
| 38 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 39 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 40 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 |
| 41 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 |
| 42 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 43 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 44 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 45 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 46 | 0.000 | 0.000 | 0.998 | 0.002 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 47 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 48 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 49 | 0.000 | 0.000 | 0.000 | 0.000 | 0.000 | 0.000 | 0.000 | 0.000 | 0.001 | 0.000 | 0.000 | 0.000 | 0.000 | 0.000 | 0.000 | 0.000 | 0.999 | 0.000 | 0.000 | 0.000 |
| 50 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 51 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 |
| 52 | 0.000 | 0.000 | 0.000 | 0.999 | 0.000 | 0.000 | 0.000 | 0.000 | 0.001 | 0.000 | 0.000 | 0.000 | 0.000 | 0.000 | 0.000 | 0.000 | 0.000 | 0.000 | 0.000 | 0.000 |
| 53 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 54 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 55 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 |
| 56 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 |
| 57 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 58 | 0.000 | 0.000 | 0.004 | 0.000 | 0.000 | 0.996 | 0.000 | 0.000 | 0.000 | 0.000 | 0.000 | 0.000 | 0.000 | 0.000 | 0.000 | 0.000 | 0.000 | 0.000 | 0.000 | 0.000 |
| 59 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 60 | 0.000 | 0.000 | 0.000 | 0.000 | 0.000 | 0.000 | 0.000 | 0.000 | 0.000 | 0.001 | 0.001 | 0.000 | 0.000 | 0.000 | 0.000 | 0.000 | 0.000 | 0.998 | 0.000 | 0.000 |
| 61 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 62 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 |
| 63 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 |
| 64 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 |
| 65 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 |
| 66 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 |
| 67 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 |
| 68 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 |
| 69 | 0.000 | 0.000 | 0.000 | 0.000 | 0.000 | 0.000 | 0.000 | 0.007 | 0.000 | 0.000 | 0.000 | 0.000 | 0.000 | 0.000 | 0.000 | 0.000 | 0.000 | 0.993 | 0.000 | 0.000 |
| 70 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 71 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 72 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 73 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 |
| 74 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 |
| 75 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 76 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 77 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 78 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 |
| 79 | 0.999 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.001 | 0.000 | 0.000 | 0.000 |
| 80 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 81 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 82 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 83 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 84 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 |
| 85 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 |
| 86 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 |
| 87 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 88 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 89 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 90 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 |
| 91 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 |
| 92 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 93 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 94 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 |
| 95 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 96 | 0.000 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 |
| 97 | 0.000 | 0.000 | 0.000 | 0.000 | 0.000 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 |
| 98 | 0.000 | 0.000 | 0.000 | 1.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 |
| 99 | 0.000 | 0.000 | 0.000 | 0.000 | 0.000 | 0.999 | 0.000 | 0.000 | 0.000 | 0.000 | 0.000 | 0.000 | 0.000 | 0.000 | 0.000 | 0.000 | 0.000 | 0.000 | 0.000 | 0.000 |
| 100 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 0.000 | 1.000 | 0.000 | 0.000 |

TABLE 5
Normalised Shannon Entropy and derived consensus sequence

| | Shannon | Normalised | | |
|---|---|---|---|---|
| Position | Entropy | Shannon Entropy | Max | Consensus |
| 1 | 0 | 0 | 1 | M |
| 2 | 0.000530494 | 0.000122745 | 0.999970516 | S |
| 3 | 0.000136309 | 3.1539E−05 | 0.999992629 | P |
| 4 | 0.000136309 | 3.1539E−05 | 0.999992629 | T |
| 5 | 0.005289368 | 0.001223844 | 0.999587219 | T |
| 6 | 0.000646496 | 0.000149585 | 0.999963145 | S |
| 7 | 0.000373878 | 8.65073E−05 | 0.999977887 | V |
| 8 | 0.006892284 | 0.001594724 | 0.999447168 | V |
| 9 | 0.006898716 | 0.001596213 | 0.999469281 | F |
| 10 | 0.006895572 | 0.001595485 | 0.99946191 | T |
| 11 | 0 | 0 | 1 | L |
| 12 | 0.001612658 | 0.000373134 | 0.999889434 | H |
| 13 | 0.000272618 | 6.30779E−05 | 0.999985258 | S |
| 14 | 0.004605877 | 0.001065699 | 0.999660929 | R |
| 15 | 0.017081508 | 0.003952289 | 0.998444698 | T |
| 16 | 0.000136309 | 3.1539E−05 | 0.999992629 | S |
| 17 | 0.000758885 | 0.000175589 | 0.999955773 | F |
| 18 | 0 | 0 | 1 | C |
| 19 | 0.002768864 | 0.000640655 | 0.999815723 | M |
| 20 | 0.00171419 | 0.000396626 | 0.999889434 | V |
| 21 | 0.002931247 | 0.000678227 | 0.99980098 | G |
| 22 | 0.000744143 | 0.000172178 | 0.999955773 | F |
| 23 | 0.001077407 | 0.000249288 | 0.99993366 | S |
| 24 | 0.003622701 | 0.000838214 | 0.999749383 | T |
| 25 | 0.000744143 | 0.000172178 | 0.999955773 | T |
| 26 | 0.000732278 | 0.000169433 | 0.999955773 | S |
| 27 | 0.001016487 | 0.000235193 | 0.99993366 | S |
| 28 | 0.002405041 | 0.000556474 | 0.999845207 | E |
| 29 | 0.00632829 | 0.001464229 | 0.999498765 | T |
| 30 | 0.04671152 | 0.010808028 | 0.995429919 | G |
| 31 | 0.000408927 | 9.46168E−05 | 0.999977887 | F |
| 32 | 0.025909774 | 0.005994957 | 0.997427487 | R |
| 33 | 0.003615713 | 0.000836597 | 0.999756754 | S |
| 34 | 0.000732278 | 0.000169433 | 0.999955773 | S |
| 35 | 0 | 0 | 1 | Q |
| 36 | 0.001152795 | 0.000266732 | 0.999926289 | A |
| 37 | 0.020190695 | 0.004671687 | 0.998090886 | R |
| 38 | 0.000732278 | 0.000169433 | 0.999955773 | L |
| 39 | 0.003610078 | 0.000835293 | 0.999749383 | S |
| 40 | 0.001675295 | 0.000387627 | 0.999889434 | |
| 41 | 0.002128405 | 0.000492466 | 0.999859949 | P |
| 42 | 0.002575282 | 0.000595864 | 0.999823094 | C |
| 43 | 0.00184147 | 0.000426076 | 0.999882062 | A |
| 44 | 0.001340109 | 0.000310072 | 0.999918918 | S |
| 45 | 0.000272618 | 6.30779E−05 | 0.999985258 | S |
| 46 | 0.021584233 | 0.004994121 | 0.998068772 | D |
| 47 | 0.001649144 | 0.000381576 | 0.999896805 | F |
| 48 | 0.002346894 | 0.00054302 | 0.999837836 | S |
| 49 | 0.010654215 | 0.002465153 | 0.999100726 | T |
| 50 | 0.002688254 | 0.000622003 | 0.999808351 | S |
| 51 | 0.005109157 | 0.001182148 | 0.999609332 | N |
| 52 | 0.008733274 | 0.002020689 | 0.99926289 | E |
| 53 | 0.000486267 | 0.000112512 | 0.999970516 | F |
| 54 | 0.001900288 | 0.000439685 | 0.99986732 | D |
| 55 | 0.000809309 | 0.000187257 | 0.999948402 | V |
| 56 | 0.00241193 | 0.000558068 | 0.999830465 | S |
| 57 | 0.001709186 | 0.000395469 | 0.999882062 | T |
| 58 | 0.042272564 | 0.00978095 | 0.995673166 | G |
| 59 | 0.000631754 | 0.000146174 | 0.999963145 | F |
| 60 | 0.025472326 | 0.005893741 | 0.997737073 | V |
| 61 | 0.004127507 | 0.000955015 | 0.999705156 | L |
| 62 | 0.000530494 | 0.000122745 | 0.999970516 | Q |
| 63 | 0 | 0 | 1 | R |
| 64 | 0 | 0 | 1 | Q |
| 65 | 0.000272618 | 6.30779E−05 | 0.999985258 | R |
| 66 | 0.001972064 | 0.000456293 | 0.999874691 | 1 |
| 67 | 0.000136309 | 3.1539E−05 | 0.999992629 | H |
| 68 | 0.000136309 | 3.1539E−05 | 0.999992629 | Q |
| 69 | 0.057740099 | 0.013359801 | 0.993410238 | V |
| 70 | 0 | 0 | 1 | F |
| 71 | 0.000257876 | 5.96669E−05 | 0.999985258 | G |
| 72 | 0.000257876 | 5.96669E−05 | 0.999985258 | L |
| 73 | 0.000257876 | 5.96669E−05 | 0.999985258 | Y |
| 74 | 0.000394185 | 9.12058E−05 | 0.999977887 | V |
| 75 | 0.002644422 | 0.000611862 | 0.999823094 | A |
| 76 | 0 | 0 | 1 | L |
| 77 | 0.000136309 | 3.1539E−05 | 0.999992629 | L |
| 78 | 0.003004165 | 0.000695098 | 0.999778867 | V |
| 79 | 0.007176519 | 0.00166049 | 0.999410312 | A |
| 80 | 0 | 0 | 1 | L |
| 81 | 0.001417421 | 0.00032796 | 0.999904176 | L |
| 82 | 0.000272618 | 6.30779E−05 | 0.999985258 | T |
| 83 | 0 | 0 | 1 | C |
| 84 | 0.000257876 | 5.96669E−05 | 0.999985258 | Q |
| 85 | 0.000136309 | 3.1539E−05 | 0.999992629 | T |
| 86 | 0.000136309 | 3.1539E−05 | 0.999992629 | 1 |
| 87 | 0.000136309 | 3.1539E−05 | 0.999992629 | G |
| 88 | 0 | 0 | 1 | L |
| 89 | 0.000136309 | 3.1539E−05 | 0.999992629 | C |
| 90 | 0.000136309 | 3.1539E−05 | 0.999992629 | N |
| 91 | 0 | 0 | 1 | N |
| 92 | 0 | 0 | 1 | L |
| 93 | 0.000732278 | 0.000169433 | 0.999955773 | A |
| 94 | 0 | 0 | 1 | P |
| 95 | 0 | 0 | 1 | F |
| 96 | 0 | 0 | 1 | L |
| 97 | 0 | 0 | 1 | K |
| 98 | 0.004471554 | 0.00103462 | 0.999683043 | E |
| 99 | 0.006910869 | 0.001599025 | 0.999491394 | G |
| 100 | 0 | 0 | 1 | V |
| Mean | 0.004114969 | 0.000952114 | | |

### Example 4-Determination of a Consensus Sequence for an ORF Encoded by the Orflab Gene of SARS-COV-2 in the Opposite Sense to Positive Sense RNA Capable of Translation

238,754 SARS-COV2 high quality genomes (i.e. complete, high coverage with any mutations, insertions or deletions verified by the submitter) were downloaded from GISAID. The genomes were downloaded irrespective of clade, sampling date, and geographic location. The longest negative sense ORF encoded by the orflab gene (i.e. the longest ORF encoded by the orflab gene of SARS-COV-2 in the opposite sense to positive sense RNA capable of translation) determined for each sequence. Out of all of these sequences 235,354 had a 100 residue negative sense ORF. These ORF sequences were analysed to determine the consensus sequence and Shannon Entropy for each position in the sequence.

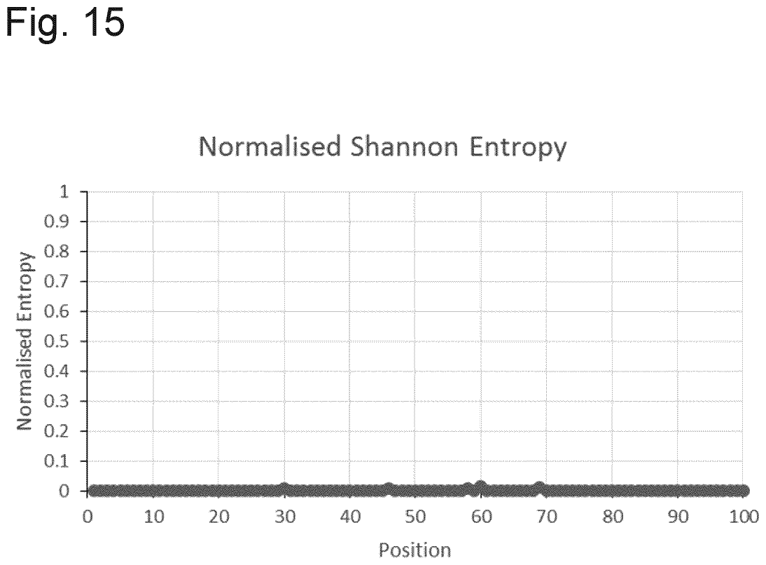

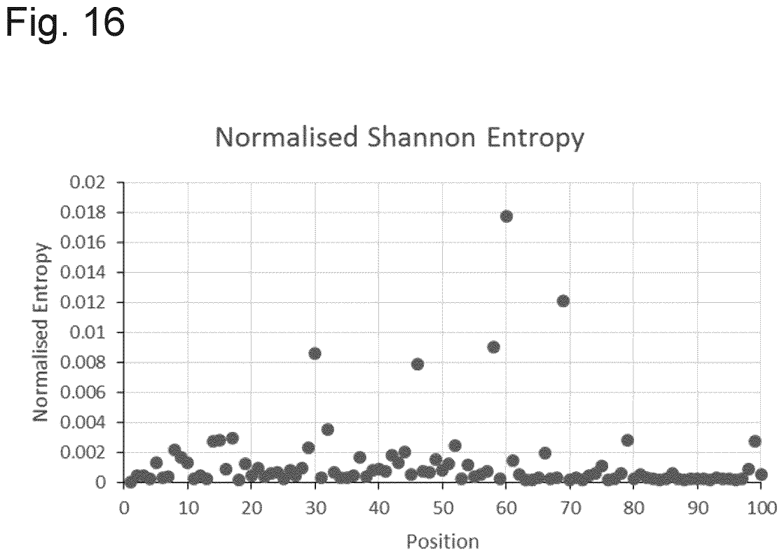

Amino acid frequencies and the derived consensus sequence (SEQ ID NO: 13) are set out in Table 6 below. As shown in FIGS. 15 ad 16, the ORF is well conserved between SARS-COV-2 strains.

TABLE 6
Prevalence of specific amino acids in ORF100

| | Amino Acid | | | | | | | | | | | |
|---|---|---|---|---|---|---|---|---|---|---|---|---|
| Pos | A | C | D | E | F | G | H | I | K | L | M | N |
| 1 | 0 | 0 | 0 | 0 | 0 | 0 | 0 | 0 | 0 | 0 | 235354 | 0 |
| 2 | 1 | 3 | 0 | 0 | 14 | 0 | 0 | 0 | 0 | 2 | 0 | 0 |
| 3 | 0 | 0 | 0 | 0 | 0 | 0 | 0 | 1 | 0 | 11 | 0 | 0 |
| 4 | 0 | 0 | 0 | 0 | 0 | 0 | 0 | 10 | 3 | 0 | 0 | 0 |
| 5 | 1 | 0 | 0 | 0 | 2 | 0 | 0 | 4 | 0 | 0 | 0 | 86 |
| 6 | 0 | 0 | 0 | 0 | 3 | 0 | 0 | 3 | 0 | 8 | 0 | 1 |
| 7 | 2 | 0 | 1 | 0 | 0 | 0 | 0 | 16 | 1 | 0 | 0 | 0 |
| 8 | 11 | 3 | 0 | 0 | 12 | 60 | 0 | 69 | 0 | 1 | 0 | 0 |
| 9 | 0 | 3 | 2 | 0 | 235227 | 3 | 0 | 3 | 0 | 107 | 0 | 0 |
| 10 | 1 | 0 | 0 | 0 | 1 | 1 | 0 | 22 | 65 | 1 | 0 | 0 |
| 11 | 0 | 0 | 0 | 1 | 0 | 0 | 2 | 3 | 1 | 235340 | 0 | 0 |
| 12 | 0 | 0 | 0 | 0 | 0 | 0 | 235324 | 1 | 2 | 0 | 0 | 3 |
| 13 | 3 | 1 | 0 | 0 | 0 | 0 | 0 | 0 | 1 | 0 | 0 | 0 |
| 14 | 0 | 0 | 0 | 0 | 0 | 0 | 0 | 4 | 211 | 0 | 0 | 1 |
| 15 | 2 | 0 | 0 | 0 | 1 | 0 | 0 | 0 | 199 | 1 | 24 | 0 |
| 16 | 1 | 1 | 0 | 0 | 24 | 0 | 0 | 2 | 0 | 0 | 0 | 0 |
| 17 | 0 | 0 | 0 | 0 | 235096 | 0 | 0 | 0 | 0 | 246 | 0 | 2 |
| 18 | 4 | 235342 | 0 | 0 | 1 | 0 | 0 | 0 | 3 | 3 | 0 | 0 |
| 19 | 0 | 0 | 0 | 0 | 0 | 0 | 1 | 80 | 2 | 4 | 235259 | 0 |
| 20 | 11 | 0 | 0 | 0 | 0 | 0 | 0 | 14 | 0 | 0 | 0 | 0 |
| 21 | 0 | 3 | 0 | 22 | 1 | 235291 | 2 | 0 | 0 | 3 | 0 | 0 |
| 22 | 0 | 1 | 0 | 0 | 235331 | 0 | 0 | 0 | 0 | 13 | 3 | 1 |
| 23 | 1 | 0 | 0 | 0 | 22 | 0 | 0 | 1 | 2 | 0 | 0 | 6 |
| 24 | 2 | 4 | 0 | 0 | 0 | 1 | 1 | 12 | 0 | 0 | 0 | 21 |
| 25 | 0 | 0 | 0 | 0 | 1 | 0 | 2 | 0 | 0 | 0 | 1 | 7 |
| 26 | 1 | 0 | 0 | 0 | 17 | 0 | 2 | 0 | 3 | 1 | 0 | 0 |
| 27 | 0 | 0 | 4 | 1 | 0 | 3 | 0 | 0 | 0 | 7 | 0 | 1 |
| 28 | 3 | 0 | 5 | 235291 | 3 | 17 | 0 | 0 | 27 | 0 | 0 | 0 |
| 29 | 1 | 3 | 1 | 2 | 0 | 0 | 0 | 1 | 0 | 0 | 0 | 187 |
| 30 | 13 | 0 | 349 | 0 | 3 | 234541 | 0 | 0 | 0 | 0 | 0 | 0 |
| 31 | 1 | 3 | 0 | 0 | 235334 | 0 | 0 | 0 | 1 | 8 | 0 | 1 |
| 32 | 0 | 1 | 1 | 0 | 0 | 0 | 0 | 3 | 305 | 3 | 0 | 4 |
| 33 | 4 | 0 | 3 | 0 | 4 | 2 | 1 | 0 | 0 | 1 | 0 | 0 |
| 34 | 2 | 0 | 0 | 0 | 1 | 0 | 2 | 0 | 0 | 9 | 0 | 0 |
| 35 | 0 | 0 | 0 | 0 | 0 | 0 | 7 | 3 | 0 | 2 | 0 | 0 |
| 36 | 235324 | 1 | 0 | 1 | 0 | 0 | 0 | 0 | 3 | 2 | 0 | 0 |
| 37 | 0 | 0 | 0 | 0 | 0 | 2 | 0 | 1 | 123 | 0 | 0 | 3 |
| 38 | 0 | 0 | 1 | 0 | 12 | 0 | 0 | 0 | 0 | 235330 | 0 | 0 |
| 39 | 5 | 1 | 0 | 0 | 0 | 0 | 0 | 0 | 0 | 1 | 0 | 3 |
| 40 | 0 | 1 | 0 | 0 | 0 | 2 | 0 | 235297 | 0 | 3 | 1 | 1 |
| 41 | 1 | 1 | 0 | 0 | 0 | 0 | 0 | 0 | 0 | 34 | 2 | 1 |
| 42 | 0 | 235212 | 0 | 0 | 1 | 0 | 0 | 0 | 3 | 0 | 0 | 0 |
| 43 | 235260 | 0 | 0 | 3 | 0 | 0 | 0 | 0 | 0 | 0 | 0 | 0 |
| 44 | 2 | 0 | 0 | 0 | 68 | 0 | 2 | 3 | 0 | 3 | 0 | 0 |
| 45 | 2 | 4 | 0 | 0 | 21 | 0 | 0 | 0 | 5 | 1 | 0 | 0 |
| 46 | 0 | 0 | 234591 | 641 | 1 | 7 | 0 | 0 | 0 | 3 | 0 | 104 |
| 47 | 0 | 1 | 1 | 0 | 235308 | 0 | 0 | 0 | 3 | 25 | 0 | 0 |
| 48 | 0 | 3 | 0 | 0 | 0 | 1 | 0 | 5 | 0 | 4 | 0 | 28 |
| 49 | 4 | 0 | 0 | 0 | 2 | 0 | 0 | 4 | 100 | 1 | 0 | 1 |
| 50 | 2 | 0 | 0 | 0 | 0 | 0 | 4 | 1 | 0 | 26 | 0 | 3 |
| 51 | 0 | 0 | 0 | 0 | 0 | 0 | 0 | 0 | 78 | 3 | 0 | 235263 |
| 52 | 2 | 0 | 7 | 235149 | 3 | 3 | 0 | 0 | 185 | 0 | 1 | 0 |
| 53 | 0 | 0 | 0 | 0 | 235339 | 0 | 0 | 0 | 0 | 7 | 0 | 0 |
| 54 | 0 | 0 | 235272 | 0 | 1 | 29 | 0 | 0 | 0 | 2 | 1 | 42 |
| 55 | 1 | 0 | 0 | 0 | 0 | 0 | 0 | 11 | 6 | 1 | 0 | 1 |
| 56 | 3 | 3 | 0 | 0 | 0 | 0 | 0 | 0 | 0 | 4 | 0 | 0 |
| 57 | 1 | 0 | 0 | 0 | 0 | 0 | 0 | 1 | 1 | 0 | 0 | 42 |
| 58 | 1 | 4 | 650 | 0 | 0 | 234474 | 0 | 0 | 0 | 0 | 0 | 0 |
| 59 | 2 | 4 | 0 | O | 235340 | 0 | 0 | 0 | 0 | 2 | 0 | 0 |
| 60 | 3 | 2 | 0 | 0 | 867 | 1 | 0 | 1 | 0 | 678 | 254 | 3 |
| 61 | 0 | 0 | 0 | 0 | 15 | 3 | 0 | 15 | 2 | 235256 | 0 | 0 |
| 62 | 1 | 0 | 0 | 0 | 0 | 2 | 22 | 1 | 4 | 2 | 0 | 0 |
| 63 | 0 | 2 | 0 | 0 | 1 | 0 | 0 | 0 | 0 | 0 | 0 | 3 |
| 64 | 1 | 0 | 1 | 0 | 0 | 0 | 0 | 0 | 0 | 4 | 1 | 2 |
| 65 | 1 | 6 | 0 | 1 | 1 | 2 | 1 | 0 | 3 | 0 | 0 | 0 |
| 66 | 1 | 3 | 0 | 0 | 0 | 0 | 1 | 235207 | 1 | 0 | 5 | 0 |
| 67 | 0 | 0 | 1 | 1 | 0 | 1 | 235342 | 3 | 0 | 3 | 0 | 0 |
| 68 | 1 | 0 | 0 | 0 | 0 | 1 | 7 | 0 | 1 | 0 | 0 | 0 |
| 69 | 2 | 1 | 0 | 0 | 1 | 0 | 0 | 1355 | 0 | 9 | 0 | 0 |
| 70 | 0 | 0 | 0 | 0 | 235344 | 0 | 0 | 1 | 0 | 2 | 0 | 0 |
| 71 | 1 | 0 | 10 | 0 | 0 | 235335 | 0 | 1 | 2 | 1 | 0 | 0 |
| 72 | 0 | 1 | 0 | 0 | 0 | 0 | 0 | 1 | 1 | 235343 | 0 | 0 |
| 73 | 0 | 0 | 0 | 0 | 1 | 1 | 16 | 3 | 1 | 3 | 0 | 1 |
| 74 | 1 | 1 | 0 | 1 | 0 | 0 | 0 | 0 | 3 | 1 | 26 | 1 |
| 75 | 235278 | 0 | 1 | 0 | 0 | 1 | 0 | 0 | 0 | 0 | 0 | 0 |
| 76 | 0 | 0 | 0 | 1 | 0 | 0 | 0 | 1 | 0 | 235343 | 0 | 0 |
| 77 | 0 | 0 | 0 | 0 | 1 | 0 | 0 | 0 | 1 | 235341 | 0 | 0 |
| 78 | 0 | 0 | 0 | 0 | 0 | 0 | 0 | 31 | 0 | 0 | 0 | 4 |
| 79 | 235110 | 2 | 0 | 3 | 2 | 0 | 0 | 0 | 0 | 1 | 0 | 0 |
| 80 | 0 | 0 | 0 | 0 | 3 | 0 | 0 | 0 | 1 | 235340 | 0 | 0 |
| 81 | 5 | 0 | 0 | 1 | 1 | 0 | 0 | 0 | 0 | 235319 | 0 | 0 |
| 82 | 3 | 2 | 0 | 0 | 0 | 0 | 0 | 3 | 3 | 1 | 0 | 0 |
| 83 | 0 | 235342 | 0 | 1 | 0 | 1 | 0 | 2 | 2 | 3 | 0 | 0 |
| 84 | 1 | 0 | 0 | 0 | 0 | 0 | 0 | 1 | 1 | 4 | 0 | 0 |
| 85 | 2 | 0 | 0 | 4 | 0 | 0 | 0 | 1 | 2 | 2 | 0 | 0 |
| 86 | 0 | 0 | 1 | 1 | 2 | 0 | 0 | 235315 | 0 | 0 | 2 | 0 |
| 87 | 0 | 0 | 3 | 0 | 3 | 235339 | 0 | 0 | 1 | 1 | 0 | 1 |
| 88 | 1 | 0 | 0 | 1 | 0 | 0 | 0 | 0 | 3 | 235345 | 1 | 0 |
| 89 | 1 | 235340 | 0 | 0 | 0 | 0 | 0 | 3 | 0 | 5 | 0 | 0 |
| 90 | 0 | 0 | 0 | 0 | 3 | 2 | 1 | 1 | 0 | 1 | 0 | 235341 |
| 91 | 0 | 0 | 0 | 0 | 0 | 0 | 0 | 2 | 0 | 2 | 0 | 235340 |
| 92 | 0 | 1 | 0 | 1 | 0 | 0 | 0 | 0 | 0 | 235344 | 0 | 0 |
| 93 | 235333 | 0 | 0 | 0 | 1 | 0 | 0 | 0 | 1 | 3 | 0 | 1 |
| 94 | 1 | 0 | 1 | 0 | 0 | 0 | 0 | 0 | 0 | 3 | 0 | 0 |
| 95 | 1 | 0 | 0 | 1 | 235340 | 1 | 0 | 0 | 4 | 3 | 0 | 0 |
| 96 | 0 | 2 | 0 | 1 | 1 | 0 | 1 | 0 | 0 | 235343 | 0 | 5 |
| 97 | 0 | 2 | 0 | 1 | 0 | 0 | 0 | 1 | 235342 | 1 | 0 | 1 |
| 98 | 0 | 3 | 5 | 235294 | 0 | 5 | 0 | 1 | 44 | 0 | 0 | 2 |
| 99 | 3 | 0 | 12 | 2 | 1 | 235130 | 0 | 1 | 0 | 2 | 1 | 1 |
| 100 | 21 | 3 | 0 | 0 | 0 | 0 | 0 | 0 | 0 | 2 | 2 | 0 |

Prevalence of specific amino acids in ORF100

| | Amino Acid | | | | | | | | | |
|---|---|---|---|---|---|---|---|---|---|---|
| | | | | | | | | | Most | |
| Pos | P | Q | R | S | T | V | W | Y | Prevalent | @ % |
| 1 | 0 | 0 | 0 | 0 | 0 | | 00 | 0 | M | 100.00 |
| 2 | 1 | 0 | 0 | 235323 | 0 | 0 | 0 | 10 | S | 99.99 |
| 3 | 235323 | 0 | 3 | 0 | 14 | 0 | 0 | 2 | P | 99.99 |
| 4 | 0 | 1 | 0 | 3 | 235337 | 0 | 0 | 0 | T | 99.99 |
| 5 | 0 | 1 | 0 | 4 | 235256 | 0 | 0 | 0 | T | 99.96 |
| 6 | 5 | 0 | 0 | 235333 | 1 | 0 | 0 | 0 | S | 99.99 |
| 7 | 0 | 0 | 3 | 0 | 0 | 235331 | 0 | 0 | V | 99.99 |
| 8 | 0 | 0 | 0 | 0 | 5 | 235193 | 0 | 0 | V | 99.93 |
| 9 | 0 | 0 | 0 | 7 | 0 | 2 | 0 | 0 | F | 99.95 |
| 10 | 0 | 0 | 3 | 3 | 235257 | 0 | 0 | 0 | T | 99.96 |
| 11 | 0 | 0 | 3 | 0 | 1 | 3 | 0 | 0 | L | 99.99 |
| 12 | 0 | 18 | 0 | 0 | 3 | 2 | 0 | 1 | H | 99.99 |
| 13 | 3 | 0 | 2 | 235340 | 3 | 0 | 1 | 0 | S | 99.99 |
| 14 | 3 | 0 | 235121 | 12 | 0 | 2 | 0 | 0 | R | 99.90 |
| 15 | 0 | 0 | 0 | 3 | 235121 | 3 | 0 | 0 | T | 99.90 |
| 16 | 7 | 0 | 3 | 235297 | 0 | 4 | 0 | 15 | S | 99.98 |
| 17 | 1 | 0 | 0 | 6 | 0 | 2 | 0 | 1 | F | 99.89 |
| 18 | 0 | 0 | 0 | 1 | 0 | 0 | 0 | 0 | C | 99.99 |
| 19 | 0 | 0 | 0 | 0 | 0 | 8 | 0 | 0 | M | 99.96 |
| 20 | 0 | 0 | 1 | 3 | 0 | 235324 | 1 | 0 | V | 99.99 |
| 21 | 0 | 0 | 31 | 0 | 0 | 0 | 0 | 1 | G | 99.97 |
| 22 | 3 | 0 | 0 | 2 | 0 | 0 | 0 | 0 | F | 99.99 |
| 23 | 4 | 0 | 1 | 235317 | 0 | 0 | 0 | 0 | S | 99.98 |
| 24 | 0 | 0 | 0 | 3 | 235310 | 0 | 0 | 0 | T | 99.98 |
| 25 | 0 | 1 | 0 | 0 | 235339 | 0 | 3 | 0 | T | 99.99 |
| 26 | 24 | 0 | 4 | 235302 | 0 | 0 | 0 | 0 | S | 99.98 |
| 27 | 9 | 0 | 2 | 235327 | 0 | 0 | 0 | 0 | S | 99.99 |
| 28 | 0 | 1 | 3 | 0 | 0 | 4 | 0 | 0 | E | 99.97 |
| 29 | 1 | 0 | 0 | 1 | 235157 | 0 | 0 | 0 | T | 99.92 |
| 30 | 0 | 0 | 1 | 443 | 1 | 3 | 0 | 0 | G | 99.65 |
| 31 | 0 | 1 | 0 | 2 | 0 | 3 | 0 | 0 | F | 99.99 |
| 32 | 0 | 0 | 235036 | 0 | 1 | 0 | 0 | 0 | R | 99.86 |
| 33 | 11 | 0 | 0 | 235311 | 3 | 0 | 0 | 14 | S | 99.98 |
| 34 | 3 | 0 | 0 | 235336 | 1 | 0 | 0 | 0 | S | 99.99 |
| 35 | 0 | 235334 | 0 | 1 | 1 | 6 | 0 | 0 | Q | 99.99 |
| 36 | 0 | 0 | 3 | 0 | 20 | 0 | 0 | 0 | A | 99.99 |
| 37 | 0 | 0 | 235219 | 0 | 4 | 1 | 0 | 1 | R | 99.94 |
| 38 | 3 | 0 | 0 | 3 | 0 | 2 | 1 | 2 | L | 99.99 |
| 39 | 25 | 0 | 0 | 235302 | 0 | 3 | 0 | 14 | S | 99.98 |
| 40 | 3 | 0 | 0 | 12 | 12 | 22 | 0 | 0 | I | 99.98 |
| 41 | 235306 | 0 | 0 | 2 | 1 | 6 | 0 | 0 | P | 99.98 |
| 42 | 0 | 1 | 28 | 109 | 0 | 0 | 0 | 0 | C | 99.94 |
| 43 | 3 | 0 | 0 | 3 | 59 | 24 | 2 | 0 | A | 99.96 |
| 44 | 67 | 0 | 0 | 235202 | 0 | 0 | 0 | 7 | S | 99.94 |
| 45 | 1 | 0 | 0 | 235319 | 1 | 0 | 0 | 0 | S | 99.99 |
| 46 | 3 | 0 | 0 | 3 | 0 | 0 | 0 | 1 | D | 99.68 |
| 47 | 0 | 1 | 0 | 5 | 0 | 7 | 3 | 0 | F | 99.98 |
| 48 | 0 | 0 | 0 | 235311 | 0 | 2 | 0 | 0 | S | 99.98 |
| 49 | 0 | 0 | 0 | 1 | 235238 | 0 | 0 | 3 | T | 99.95 |
| 50 | 19 | 0 | 0 | 235299 | 0 | 0 | 0 | 0 | S | 99.98 |
| 51 | 0 | 0 | 0 | 1 | 4 | 2 | 0 | 3 | N | 99.96 |
| 52 | 0 | 0 | 0 | 3 | 1 | 0 | 0 | 0 | E | 99.91 |
| 53 | 0 | 0 | 0 | 0 | 7 | 0 | 1 | 0 | F | 99.99 |
| 54 | 0 | 0 | 0 | 0 | 7 | 0 | 0 | 0 | D | 99.97 |
| 55 | 0 | 0 | 3 | 0 | 0 | 235331 | 0 | 0 | V | 99.99 |
| 56 | 22 | 0 | 3 | 235319 | 0 | 0 | 0 | 0 | S | 99.99 |
| 57 | 0 | 0 | 0 | 2 | 235302 | 5 | 0 | 0 | T | 99.98 |
| 58 | 1 | 0 | 0 | 219 | 0 | 2 | 3 | 0 | G | 99.63 |
| 59 | 0 | 0 | 0 | 5 | 0 | 0 | 0 | 1 | F | 99.99 |
| 60 | 0 | 0 | 0 | 1 | 0 | 233544 | 0 | 0 | V | 99.23 |
| 61 | 26 | 0 | 31 | 0 | 0 | 6 | 0 | 0 | L | 99.96 |
| 62 | 3 | 235318 | 0 | 0 | 1 | 0 | 0 | 0 | Q | 99.98 |
| 63 | 0 | 3 | 235344 | 1 | 0 | 0 | 0 | 0 | R | 100.00 |
| 64 | 0 | 235342 | 0 | 0 | 3 | 0 | 0 | 0 | Q | 99.99 |
| 65 | 2 | 0 | 235336 | 0 | 0 | 1 | 0 | 0 | R | 99.99 |
| 66 | 1 | 0 | 0 | 1 | 25 | 109 | 0 | 0 | I | 99.94 |
| 67 | 1 | 1 | 0 | 1 | 0 | 0 | 0 | 0 | H | 99.99 |
| 68 | 1 | 235336 | 1 | 0 | 2 | 1 | 3 | 0 | Q | 99.99 |
| 69 | 0 | 0 | 0 | 0 | 2 | 233984 | 0 | 0 | V | 99.42 |
| 70 | 1 | 1 | 1 | 1 | 3 | 0 | 0 | 0 | F | 100.00 |
| 71 | 0 | 2 | 0 | 2 | 0 | 0 | 0 | 0 | G | 99.99 |
| 72 | 0 | 0 | 0 | 7 | 1 | 0 | 0 | 0 | L | 100.00 |
| 73 | 0 | 0 | 1 | 0 | 0 | 0 | 0 | 235327 | Y | 99.99 |
| 74 | 0 | 0 | 0 | 3 | 0 | 235317 | 0 | 0 | V | 99.98 |
| 75 | 0 | 0 | 1 | 10 | 53 | 9 | 0 | 1 | A | 99.97 |
| 76 | 2 | 1 | 0 | 0 | 1 | 5 | 0 | 0 | L | 100.00 |
| 77 | 0 | 0 | 1 | 3 | 2 | 5 | 0 | 0 | L | 99.99 |
| 78 | 1 | 0 | 1 | 0 | 3 | 235314 | 0 | 0 | V | 99.98 |
| 79 | 0 | 0 | 1 | 1 | 231 | 3 | 0 | 0 | A | 99.90 |
| 80 | 1 | 0 | 0 | 8 | 0 | 1 | 0 | 0 | L | 99.99 |
| 81 | 1 | 0 | 0 | 25 | 1 | 1 | 0 | 0 | L | 99.99 |
| 82 | 1 | 0 | 1 | 2 | 235337 | 1 | 0 | 0 | T | 99.99 |
| 83 | 0 | 0 | 3 | 0 | 0 | 0 | 0 | 0 | C | 99.99 |
| 84 | 0 | 235343 | 0 | 0 | 1 | 3 | 0 | 0 | Q | 100.00 |
| 85 | 0 | 0 | 1 | 0 | 235342 | 0 | 0 | 0 | T | 99.99 |
| 86 | 0 | 0 | 3 | 0 | 29 | 0 | 0 | 1 | I | 99.98 |
| 87 | 1 | 3 | 0 | 1 | 0 | 1 | 0 | 0 | G | 99.99 |
| 88 | 0 | 1 | 1 | 0 | 1 | 0 | 0 | 0 | L | 100.00 |
| 89 | 0 | 0 | 2 | 0 | 0 | 0 | 0 | 3 | C | 99.99 |
| 90 | 1 | 0 | 0 | 3 | 1 | 0 | 0 | 0 | N | 99.99 |
| 91 | 0 | 0 | 2 | 4 | 1 | 3 | 0 | 0 | N | 99.99 |
| 92 | 0 | 1 | 3 | 2 | 1 | 0 | 0 | 1 | L | 100.00 |
| 93 | 1 | 0 | 3 | 0 | 11 | 0 | 0 | 0 | A | 99.99 |
| 94 | 235342 | 0 | 1 | 4 | 1 | 1 | 0 | 0 | P | 99.99 |
| 95 | 0 | 0 | 0 | 1 | 0 | 3 | 0 | 0 | F | 99.99 |
| 96 | 0 | 0 | 0 | 1 | 0 | 0 | 0 | 0 | L | 100.00 |
| 97 | 1 | 0 | 1 | 1 | 3 | 0 | 0 | 0 | K | 99.99 |
| 98 | 0 | 0 | 0 | 0 | 0 | 0 | 0 | 0 | E | 99.97 |
| 99 | 0 | 0 | 0 | 195 | 0 | 6 | 0 | 0 | G | 99.90 |
| 100 | 0 | 0 | 0 | 3 | 2 | 235320 | 1 | 0 | V | 99.99 |

The derived consensus sequence

(MSPTTSVVFTLHSRTSFCMVGFSTTSSETGFRSSQARLSIPCASSDFSTSNEFDVST GFVLQRQRIHQVFGLYVALLVALLTCQTIGLCNNLAPFLKEGV: SEQ ID NO: 13) is identical to the consensus sequence derived from SARS-COV-2 UK strains in Example 3.

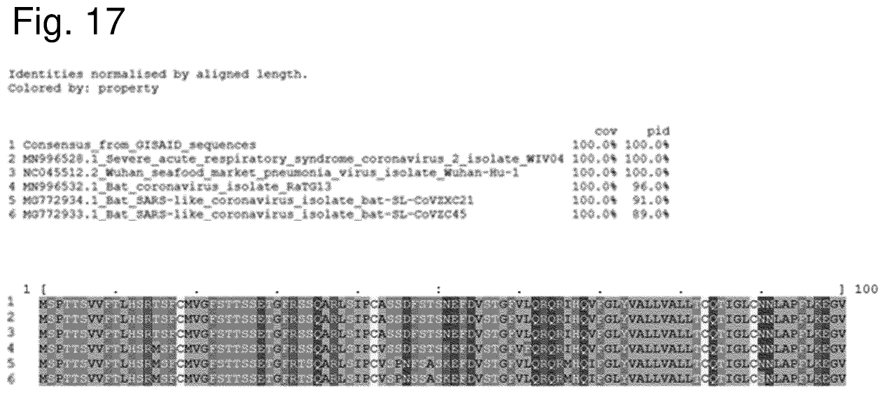

A 100 amino acid ORF identical to SEQ ID NO: 13 (SEQ ID NO: 14) is also encoded by the genome of severe acute respiratory syndrome coronavirus 2 isolate WIV04 (GenBank: MN996528.1), in the opposite sense to positive sense RNA capable of translation. A 100 amino acid ORF identical to SEQ ID NO: 13 (SEQ ID NO: 15) is also encoded by the genome of ‘Wuhan seafood market pneumonia virus’ (SARS-COV-2) isolate Wuhan-Hu-1 (GenBank: NC045512.2), in the opposite sense to positive sense RNA capable of translation. In addition, a 100 amino acid ORF having 96% sequence identity to SEQ ID NO: 13 (SEQ ID NO: 16) is encoded by the genome of bat coronavirus isolate RaTG13 (GenBank: MN996532.1), in the opposite sense to positive sense RNA capable of translation. A 100 amino acid ORF having 91% sequence identity to SEQ ID NO: 13 (SEQ ID NO: 17) is encoded by the genome of bat SARS-like coronavirus isolate bat-SL-CoVZXC21 (GenBank: MG772934.1), in the opposite sense to positive sense RNA capable of translation. A 100 amino acid ORF having 29% sequence identity to SEQ ID NO: 13 (SEQ ID NO: 18) is encoded by the genome of bat SARS-like coronavirus isolate bat-SL-CoVZC45 (GenBank: MG772933.1), in the opposite sense to positive sense RNA capable of translation. FIG. 17 shows an alignment of the consensus sequence (SEQ ID NO: 13) with these early SARS-COV-2 and bat-coronavirus ORF100 sequences.

### “Outlier” Sequences

Due to the sheer number of outlier sequences (3,400 out of 238,754, 1.4% of total downloaded sequences did not generate a longest negative sense ORF of 100 residues length) it would not be feasible to manually analyse each sequence. Duplicate longest negative sense ORF sequences were excluded, so were similar sequences of the same length. From these 244 sequences 24 were picked at random and the ORF100 consensus sequence aligned with the genomic data using Exonerate. A cursory glance at the genomic data showed several stretches of unassigned bases in several of the sequences looked at, the LNSORF script may need to be adjusted to cope with misreads and ambiguous bases.

TABLE 7
Summary of Exonerate alignments with “outlier” sequences.

| | Normalised | |
|---|---|---|
| Name | Exonerate Score | Notes |
| hCoV-19/England/20129035804/2020\|EPI_ISL_424031\|2020-03-20 | 100 | |
| hCoV-19/Australia/VIC9811/2020\|EPI_ISL_565730\|2020-08-12 | 96 | Ms in ORF100 |
| | | region |
| hCoV-19/England/BIRM-5E346/2020\|EPI_ISL_444288\|2020-04-15 | 99 | N in ORF100 |
| | | region |
| hCoV-19/USA/TX-HMH-7736/2020\|EPI_ISL_545608\|2020-07-02 | 100 | |
| hCoV-19/England/204691396/2020\|EPI_ISL_686598\|2020-11-14 | 99 | N in ORF100 |
| | | region |
| hCoV-19/Australia/VIC1410/2020\|EPI_ISL_456429\|2020-04-16 | 100 | |
| hCoV-19/Australia/VIC14728/2020\|EPI_ISL_592972\|2020-08-11 | 100 | |
| hCoV-19/India/MH-NIV-9127/2020\|EPI_ISL_454556\|2020-04-12 | 94 | Ns in ORF100 |
| | | region |
| hCoV-19/Gambia/GC19-3812/2020\|EPI_ISL_561086\|2020-07-27 | 100 | |
| hCoV-19/Switzerland/BE-ETHZ-410879/2020\|EPI_ISL_737743\|2020-12-14 | 100 | R in ORF100 |
| | | region |
| hCoV-19/England/LIVE-A5F1F/2020\|EPI_ISL_499668\|2020-03-21 | 100 | |
| hCoV-19/USA/NY-NYUMC722/2020\|EPI_ISL_451437\|2020-03-26 | 100 | |
| hCoV-19/USA/CA-IGI-0760/2020\|EPI_ISL_677885\|2020-07-30 | 100 | |
| hCoV-19/Australia/VIC1340/2020\|EPI_ISL_430686\|2020-04-13 | 100 | |
| hCoV-19/Croatia/17-S7/2020\|EPI_ISL_429802\|2020-03 | 100 | |
| hCoV-19/Switzerland/ZH-ETHZ-230073/2020\|EPI_ISL_516606\|2020-08-06 | 100 | |
| hCoV-19/South Africa/KRISP-0312/2020\|EPI_ISL_487295\|2020-06-24 | 100 | |
| hCoV-19/USA/CA-IGI-0710/2020\|EPI_ISL_677877\|2020-07-24 | 100 | |
| hCoV-19/USA/NY-NYUMC651/2020\|EPI_ISL_444768\|2020-04-06 | 100 | |
| hCoV-19/England/20134072104/2020\|EPI_ISL_423164\|2020-03-25 | 100 | |
| hCoV-19/Switzerland/SZ-ETHZ-310400/2020\|EPI_ISL_603474\|2020-10-08 | 100 | R in ORF100 |
| | | region |
| hCoV-19/England/OXON-AEC88/2020\|EPI_ISL_448775\|2020-04-07 | 100 | |
| hCoV-19/England/MILK-975489/2020\|EPI_ISL_552613\|2020-08-21 | 100 | |
| hCoV-19/England/20099071404/2020\|EPI_ISL_464218\|2020-02-29 | 100 | |

All of the outlier sequences analysed contained the ORF100 sequence with the cause of the misidentification being due to the presence of sequencing gaps and the presence of ambiguous bases in the sequence analysed.

### Example 5-Stimulation of Primary Human Peripheral Blood Mononuclear Cells with a Panel of Negative Sense SARS-COV-2 Peptides and Staining with a SARS-COV-2 Negative Sense Peptide MHC Dextramer

### SUMMARY

A panel of 7 negative sense SARS-COV-2 peptides were used to stimulate human PBMCs from internal blood donors (EVB00028, EVB00031, EVB00033). Cells were subsequently analysed for the production of IFN-gamma, and a variety of T cell markers.

### Methods

### A) PBMC Preparation

Informed consent was obtained from blood donors prior to venipuncture. Donors were assigned an internal number. 60 mL of blood was drawn from consented volunteers into EDTA tubes. Whole blood was diluted in buffer (PBS+1% bovine serum albumin) as follows: 30 mL blood+20 mL buffer.

25 mL of diluted blood was carefully layered onto 18 mL of Ficoll-paque solution in 50 mL falcon tubes. Blood samples were centrifuged for 20 minutes at room temperature at 300×g, deceleration set at 0.

Plasma was carefully removed and dispense into a clean 50 mL falcon tube, labelled with the donor's number, and stored at −800° C. until further analysis. Buffy coat (PBMC) layer was carefully removed and dispensed into a clean 50 mL falcon tube. Cell samples were topped up to 50 mL with buffer, centrifuged for 10 mins, 40C, 300×g. Supernatants were discarded, cell pellet was resuspended in 50 mL buffer and centrifuged as above. This was repeated one more time. Cells were then counted and the resuspended at 2.5×10\hat{ }6/mL in AB medium (500 mL RPMI 1640 supplemented with L-Glutamine, 50 mL heat-inactivated AB serum, 5 mL Penicillin/Streptomycin, 5 mL (200 g/L) Glucose.

### B) Peptide Stimulations

Cells were then aliquoted in 1 mL volume into wells of a 24-well plate.

AB media with 50 IU IL-2 was prepared. Each of the following peptides diluted to 20 μM concentration in AB media with 50 IU IL-2:

(SEQ ID NO: 4)
LSIPCASSDFST
(SEQ ID NO: 6)
LYVALLVALLTCQ
(SEQ ID NO: 8)
RLSIPCASSDF
(SEQ ID NO: 9)
TCQTIGLCNNLAPFL
(SEQ ID NO: 10)
TLHSRTSFCMV
(SEQ ID NO: 11)
VALLTCQTIGLCN
(SEQ ID NO: 12)
VFTLHSRTSF

Diluted peptides were added to the PBMCs and plates placed in an incubator at 37° C., 5% CO₂. Cells were fed by semi-depletion at days 2, 4 and day 6. Briefly, 1 mL of media was removed from each well and supplemented with 1 mL of fresh AB media with 50IU IL-2.

### C) ELISPOT Preparation

Serum-free ELISPOT media was prepared (500 mL RPMI 1640+1% v/v Penicillin/Streptomycin). Human IFN-γ ELISPOT plate (Mabtech) was washed 4 times with PBS and blocked with AB-media for at least 30 minutes, room temperature. PBMCs were harvest, centrifuged for 10 mins, 300×g, 40° C., resuspended in serum-free media and counted. Cells were re-suspended at 5×10\hat{ }6 cells/mL. PBMCs were added to wells in the ELISPOT well at 50 μL per well.

Peptides were prepared for re-stimulating PBMCs at a concentration of 400 μM and added at 50 μL volume to ELISPOT wells, for final concentration of 200 μM in wells.

Plates were incubated for 24 hours at 37° C., 5% CO₂ with lids and wrapped in foil.

After 24 hours, ELISPOT plates were developed according to Mabtech protocol.

After drying, plates were scanned using a CTL Immunospot analyser, spots counted and quality controlled and data plotted as bar charts.

D) Flow Cytometry Analysis of T Cell Markers

At day 7 post-stimulation, remaining PBMCs were resuspended in flow cytometry buffer (PBS+1% BSA+2 mM EDTA) at a concentration of 1×10\hat{ }6 cells/mL, and blocked for at least 10 minutes. Cells were then aliquoted in a 96-well plate, centrifuged (5 mins/40° C./300×g), and resuspended in 45 μL buffer. Live/Dead stain was added (Fixable Near IR) at 1 μL in 1000 μL, and cells incubated at 40° C. for 10 minutes in the dark. 120 μL buffer was added, samples were centrifuged for 5 mins, supernatants discarded and wash repeated with 200 μL buffer. Cells were then resuspended in buffer containing antibodies to T cell markers at the appropriate concentration (final staining volume 50 μL) and incubated for at least 30 minutes in the dark at 40° C.

TABLE 8
Summary of antibodies used in T cell flow cytometry panel

| | | |
|---|---|---|
| Marker/ | Detection | Volume |
| Fluorochrome | channel | (μl) |

| | | | |
|---|---|---|---|
| CD25 | Alexa Fluor 488 | BL1 | 5 |
| CD4 | PerCP-eFluor710 | BL2 | 5 |
| OX40 | Alexa Fluor 405 | VL1 | 5 |
| CD45RA | eFLuor 506 | VL2 | 1 |
| CD69 | Super Bright 600 | VL3 | 2 |
| CD197 | Super Bright 645 | VL4 | 1 |
| (CCR7) | | | |
| CD38 | Super Bright 702 | VL5 | 5 |
| CD103 | Super Bright 780 | VL6 | 2 |
| CD45RO | PE | YL1 | 1 |
| CD29 | PE eFluor610 | YL2 | 1 |
| CD3 | PE-Cyanine7 | YL3 | 2 |
| PDL-1 | APC | RL1 | 5 |
| CD8a | Alexa Fluor 700 | RL2 | 1 |
| Live Dead | NIR | RL3 | |

TABLE 9
Definition of cell surface markers.

| Marker | Definition |
|---|---|
| CD25 | Activation induced marker |
| CD4 | Helper T cell marker |
| CD45RA | Naïve T cell marker |
| CD69 | Early activation marker |
| CD197 | Naïve T cell marker |
| CD38 | Activation marker |
| CD103 | Resident memory T cell marker if co-expressed CD69 |
| CD45RO | Memory T cell marker |
| CD29 | IFN-gamma producing cytotoxic T cells |
| CD3 | Pan T cell marker |
| PDL-1 | Activation induced marker |
| CD8a | Cytotoxic T cell marker |

120 μL buffer was added to cell samples, centrifuged for 5 mins, supernatants discarded. This wash step was then repeated. Compensation beads were incubated with the individual antibodies at the same concentration. Voltages on the Attune flow cytometer were adjusted using antibody-stained compensation beads, as well as unstained and NIR-stained cells.

Acquisition was set to acquire at least 150,000 events on CD3 gated cells.

### E) Flow Cytometry Analysis of Dextramer-Stained Samples

PBMCs were prepared as described in previous sections. Cells were counted and prepared 10-50×10\hat{ }6/ml. Cells were resuspended in PBS containing 5% fetal calf serum and 100 μL cell suspension was added per well of a U-bottom 96-well plate. Cells were stained with Live/Dead stain Fixable Near IR as previously described, before the Immudex staining protocol was followed. 10 μL MHC Dextramer was added to samples, mixed thoroughly and incubated in the dark at room temperature for 10 min. Samples were washed five times by adding 200 μL buffer, centrifuging for 5 minutes, 40° C. Samples were then stained for CD4, CD8 and CD3 cell surface markers as previously described. Samples were washed twice by resuspending in 200 μL flow cytometry buffer and analysed on the Attune flow cytometer.

MHC Dextramer used: A*2402/VFTLHSRTSF-APC

### F) HLA-Typing (Proimmune)

Cell pellets from PBMC samples were sent frozen to ProImmune Ltd for HLA-typing.

### G) Binding Affinity Prediction

In order to predict binding affinities of individual peptides to MHC molecules, based on the HLA profile of individual donors, KD values were obtained using the algorithm PSS MHC Pan.

### Results

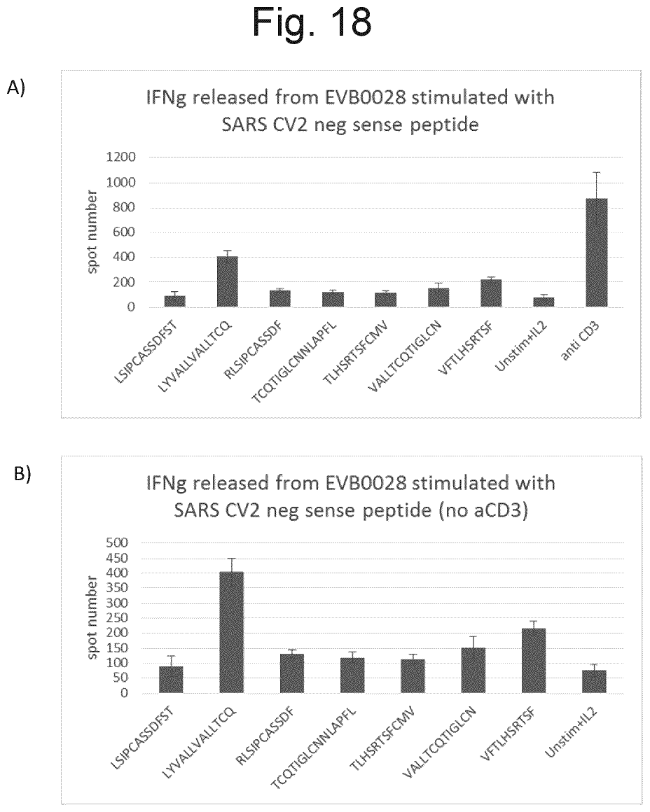

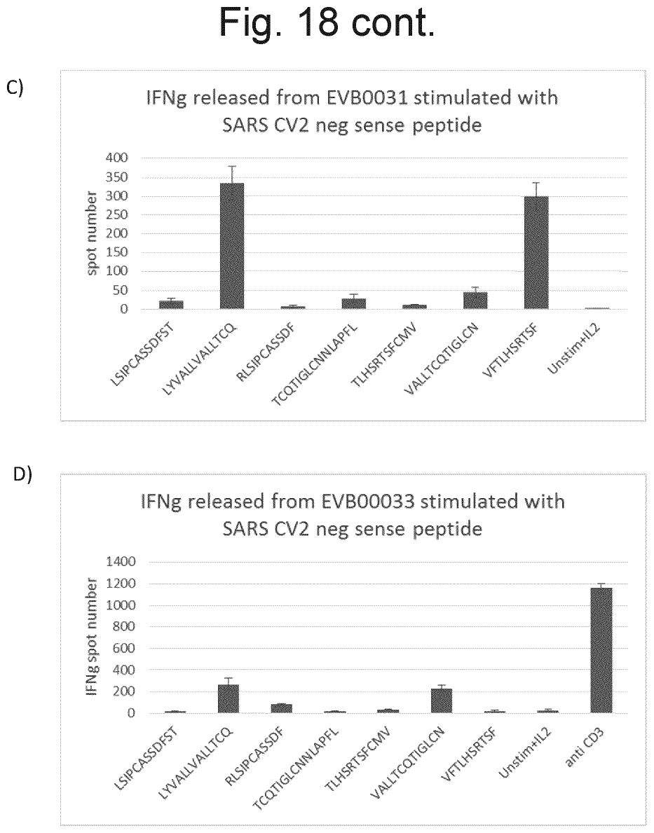

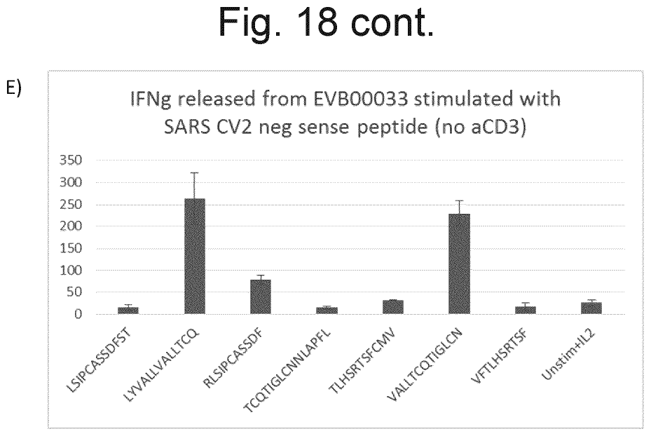

FIG. 18 shows ELISPOT data from stimulations of PBMCs (from donors EVB00028, EVB00031 and EVB00033) with negative sense SARS-COV-2 peptides. PBMCs were incubated with individual peptides for 7 days as described in the Methods section. Cells were then used in an IFN-gamma ELISPOT assay, where they were re-stimulated with the same peptide for 24 hours. Plates were developed and spots counted using a CTL Immunospot Analyser, which were plotted as mean of triplicate values-/+standard deviation. (A) and (B) show results from EVB00028 with (A) and without (B) anti-CD3 antibody, used as a positive control. (C) shows results from EVB00031. (D) and (E) show results from EVB00033 with (D) and without (E) anti-CD3 antibody, which was used as a positive control.

Results from all three subject donors showed strong reactivity to the peptide LYVALLVALLTCQ. EVB00028 and EVB00031 also reacted strongly to the peptide VFTLHSRTSF, whereas EVB00033 also reacted strongly to VALLTCQTIGLCN.

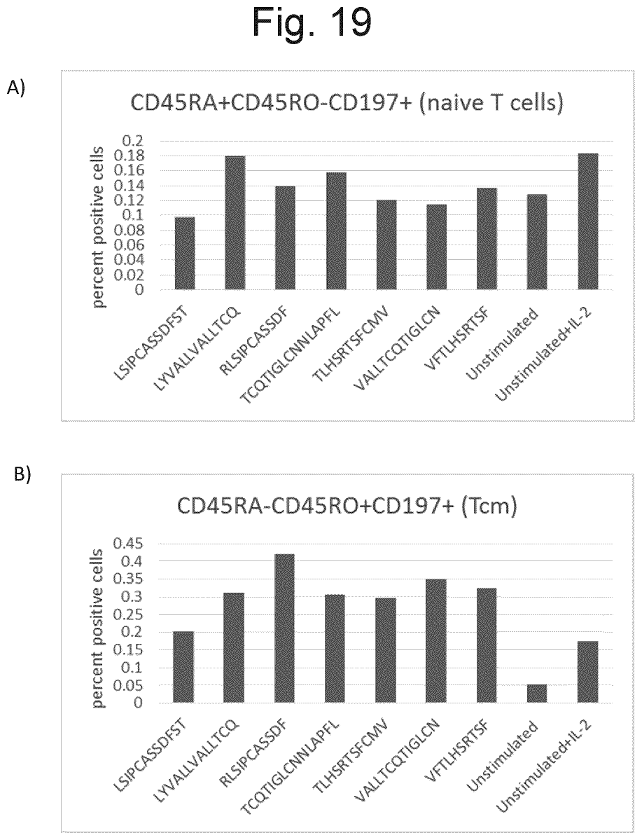

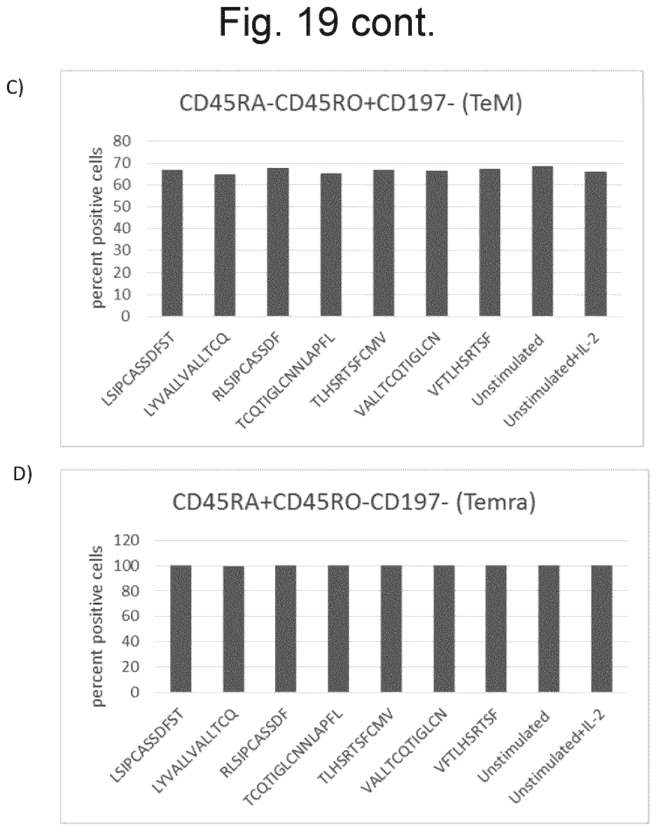

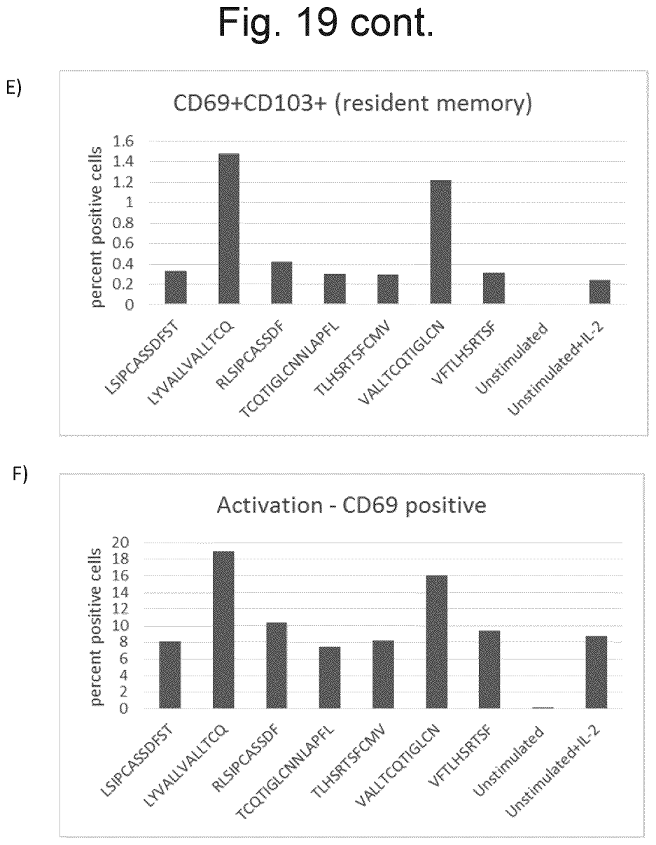

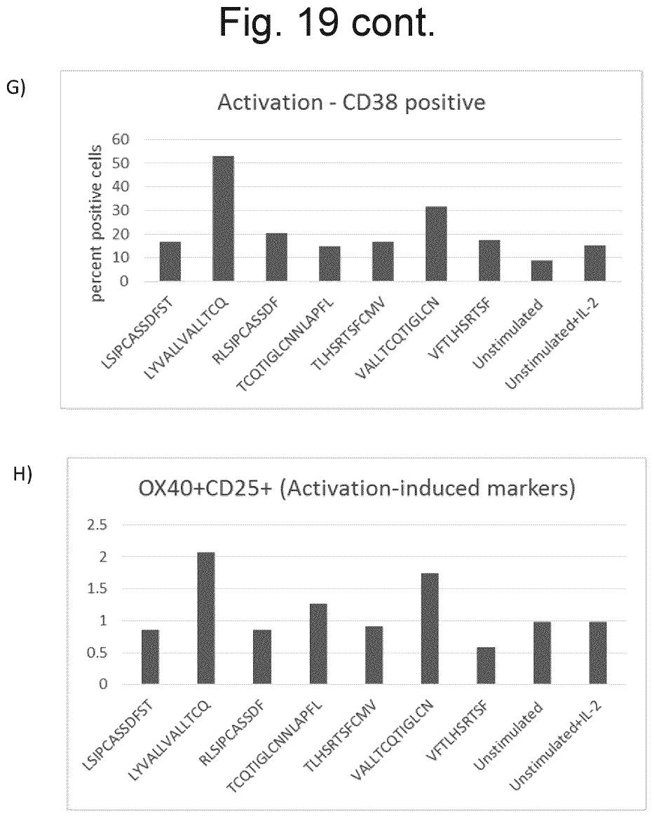

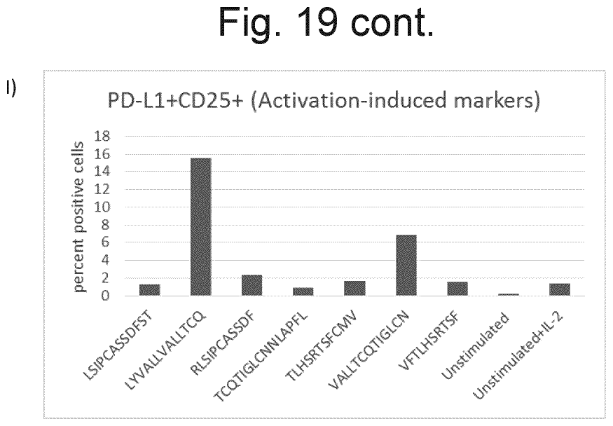

FIG. 19 shows flow cytometry analysis of T cell surface markers after stimulation of PBMCs from EVB00028. PBMCs were stained with a panel of antibodies (as listed in the previous section) against cell surface markers to characterize different T cell populations, such as naïve T cells (A), Central memory T cells (B), Effector memory T cells (C) and (D), and resident memory T cells (E). In addition, activation markers were also analysed (F) to (I). The percentage of positive cells gated on CD3+T cell populations were plotted.

Similar to the results obtained in the IFN-gamma ELISPOT assays, the peptides LYVALLVALLTCQ and VALLTCQTIGLCN induced activation in CD3+ cells from EVB00028, as shown in the upregulation of CD69, CD38, OX40/CD25, PDL1/CD25.

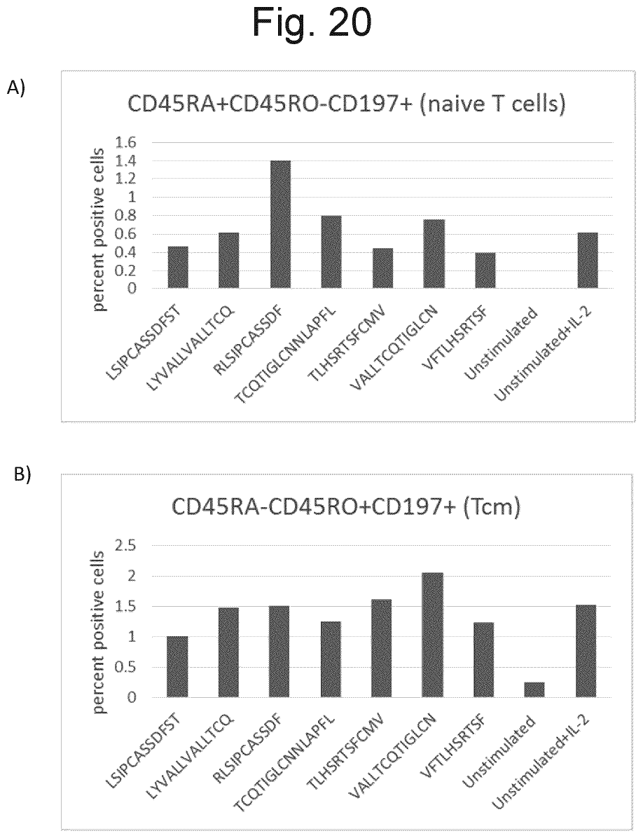

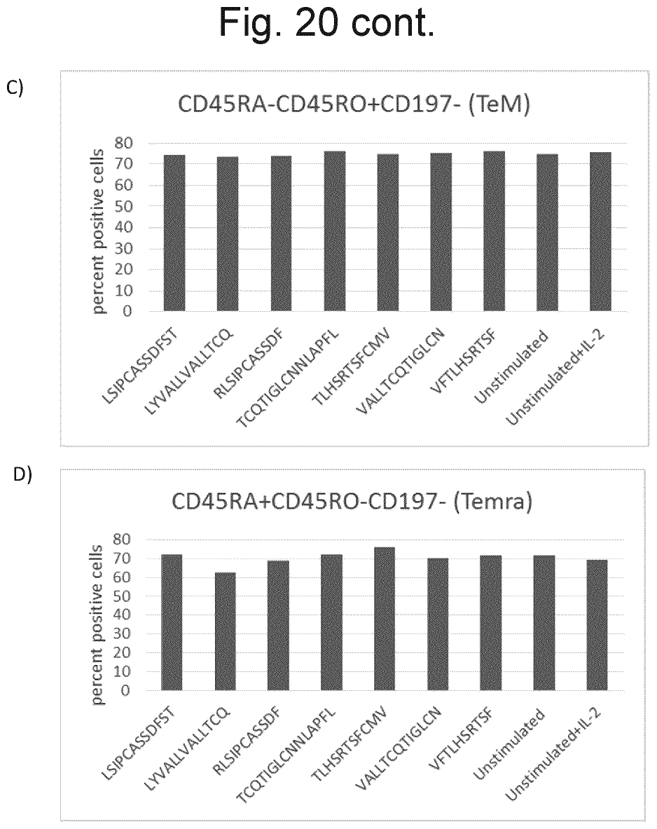

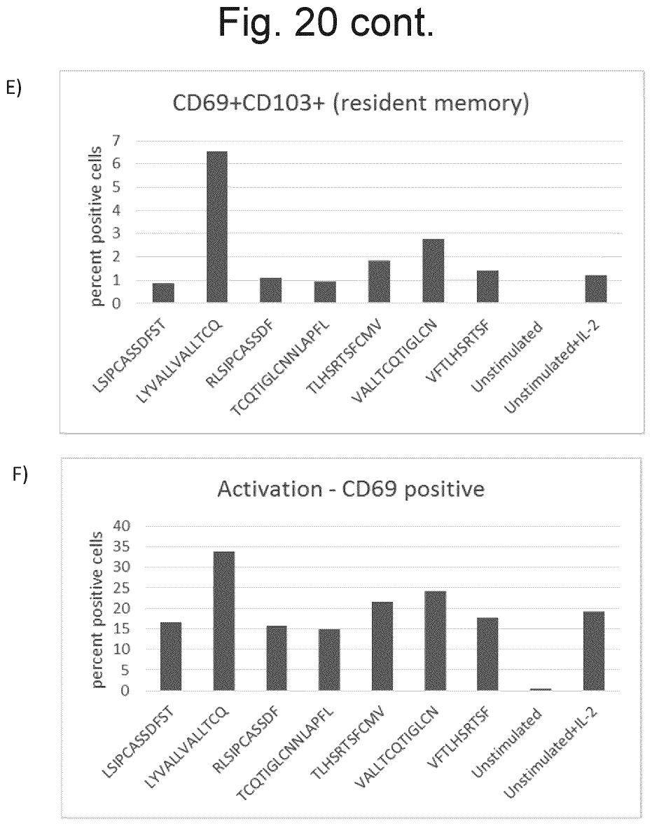

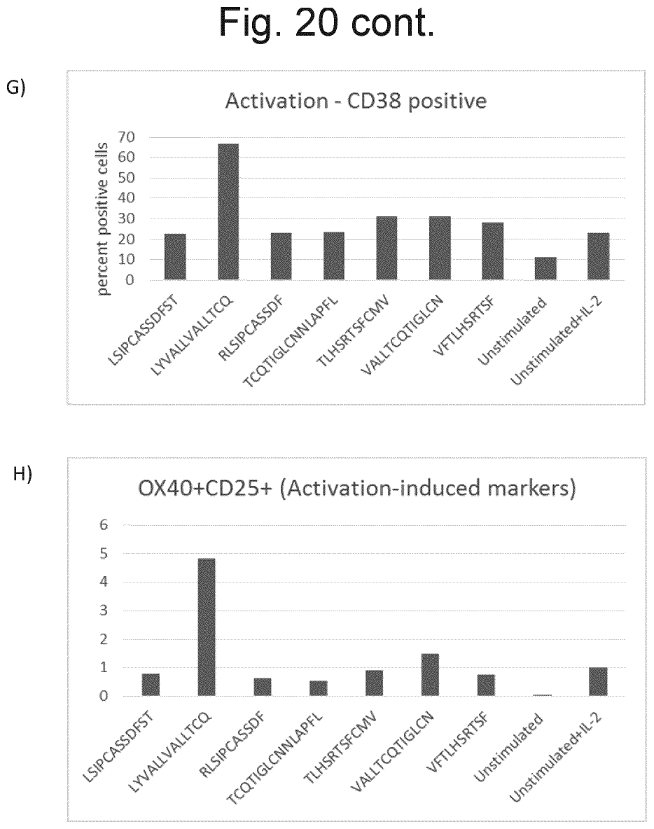

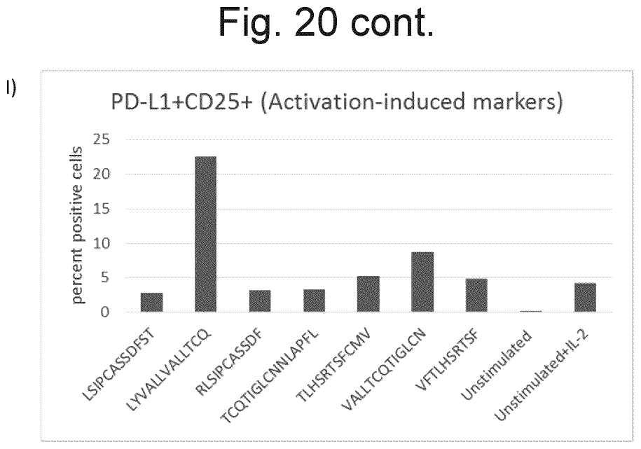

FIG. 20 shows flow cytometry analysis of T cell surface markers after stimulation of PBMCs from EVB00033. PBMCs were stained with a panel of antibodies (as listed in the previous section) against cell surface markers to characterize different T cell populations, such as naïve T cells (A), Central memory T cells (B), Effector memory T cells (C) and (D), and resident memory T cells (E). In addition, activation markers were also analysed (F) to (I). The percentage of positive cells gated on CD3+T cell populations were plotted.

CD3+ cells from EVB00033 were similarly activated by the peptides LYVALLVALLTCQ and VALLTCQTIGLCN, although to a much lesser extent with the latter, as shown in the upregulation of CD69, CD38, OX40/CD25, PDL1/CD25.

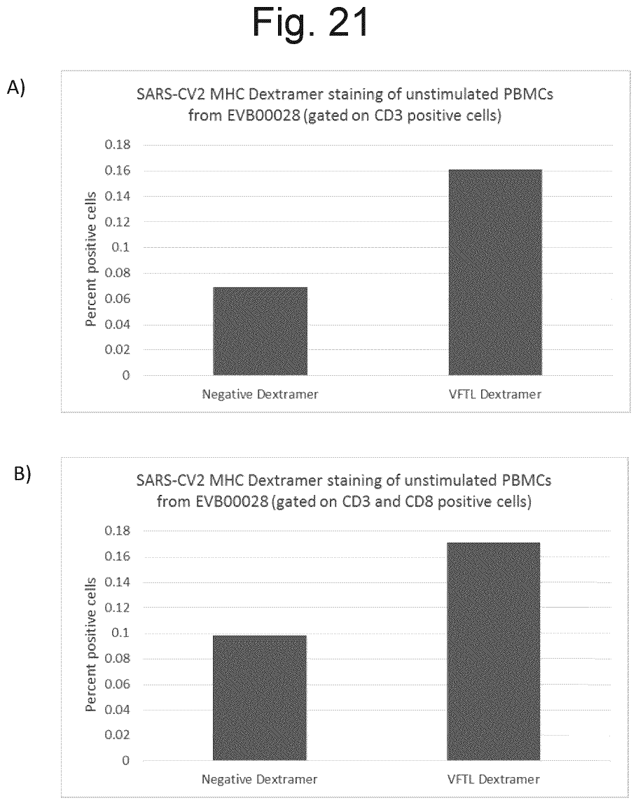

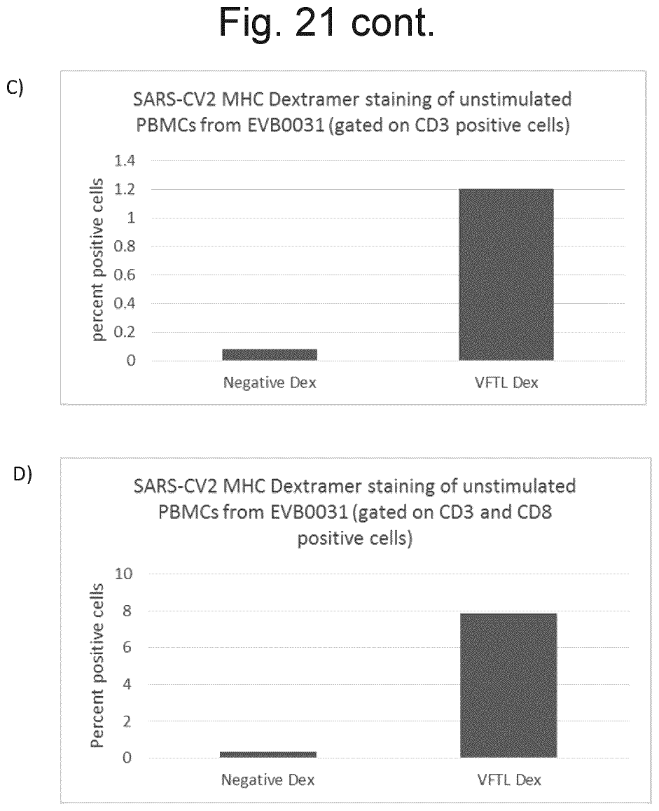

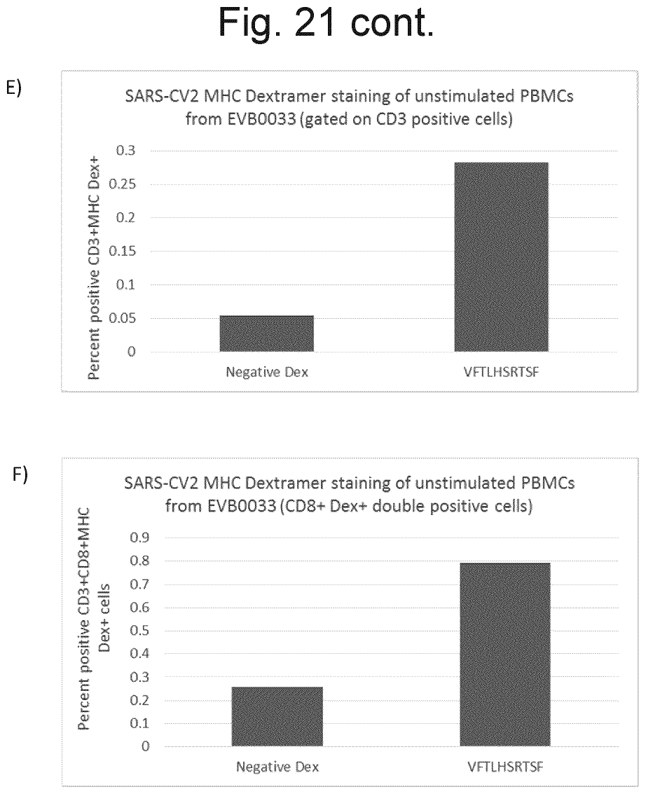

FIG. 21 shows A*2402/VFTLHSRTSF-APC MHC Dextramer staining analysis.

Unstimulated PBMCs from EVB00028, EVB00031 and EVB00033 were prepared according to the protocol previously described. PBMCs were then incubated either with VFTLHSRTSF-APC or a negative control dextramer, followed by antibodies to CD3, CD4 and CD8. Cells were either gated on CD3+ cells only or on CD3+ and CD8+ cells, and the percentage of cells positive for VFTLHSRTSF-APC plotted as bar charts. PBMCs from all 3 subjects showed the presence of CD8+ T cells that were able to bind to VFTLHSRTSF-APC dextramer.

However, as full HLA-typing results (Table 10), showed only EVB00033 having HLA-A24: 02, the question arises whether some unspecific binding of the T-cell receptor to the MHC-Dextramer is occurring.

TABLE 10
Summary of HLA-typing results of
EVB00028, EVB00031, EVB00033.

| | HLA-A | | HLA-B | | HLA-C | |
|---|---|---|---|---|---|---|
| EVB00028 | 11:01 | 11:01 | 38:02 | 40:02 | 03:03 | 07:02 |
| EVB00031 | 03:01 | 30:02 | 08:01 | 57:03 | 07:01 | 18:01 |
| EVB00033 | 02:01 | 24:02 | 07:02 | 57:01 | 06:02 | 07:02 |

TABLE 11
Summary of binding affinities of individual negative sense SARS-CoV-2
peptides (as denoted by the first 3 amino acids) to MHC molecules
shown in Table 10.

| | Predicted Kd (nM) HLA-A | | | Predicted Kd (nM) HLA-B | | | Predicted Kd (nM) HLA-C | | |
|---|---|---|---|---|---|---|---|---|---|
| Peptide | EVB00028 | EVB00031 | EVB00033 | EVB00028 | EVB00031 | EVB00033 | EVB00028 | EVB00031 | EVB00033 |
| LSI | na/na | na/na | 336/na | na/1376 | na/291 | na/291 | na/na | na/na | na/na |
| LYV | na/na | na/na | 860/na | na/1524 | na/na | na/na | na/na | na/na | na/na |
| RLS | 945/945 | 704/795 | 1250/326 | na/7210 | na/1411 | 402/2087 | na/na | na/na | na/na |
| TCQ | na/na | na/na | 1475/na | na/na | na/na | na/na | na/na | na/na | na/na |
| TLH | 5239/5239 | 4112/129 | 154/1756 | na/16444 | na/25363 | 1006/50000 | na/na | na/na | na/na |
| VAL | na/na | na/na | 546/na | na/777 | na/na | na/na | na/na | na/na | na/na |
| VFT | 3229 | 11160/1012 | 37166/23 | na/13186 | 3231/1793 | 2018/1720 | na/na | na/na | na/na |

### Example 6-Preparation of a Polynucleotide Encoding ORF100

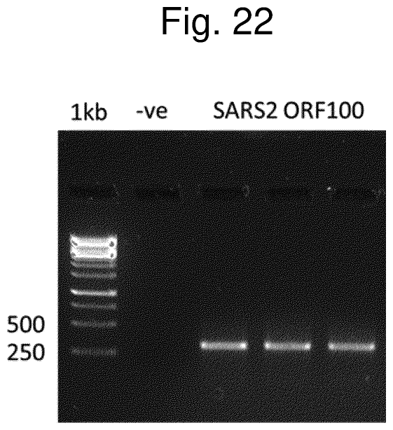

A polynucleotide sequence encoding the ORF100 consensus amino acid sequence (SEQ ID NO: 13) was synthesised using overlapping oligonucleotides using a method outlined in BioTechniques 69, 211 (incorporated herein by reference in its entirety) adapted for 21 bp overlaps and 100 bp sequence length. FIG. 22 shows banding associated with a polynucleotide of the expected size for a polynucleotide encoding ORF100.

The following oligos were used in the method:

CVNS100_FO1
Forward Oligo 1 (SEQ ID NO: 19):
atgtctcctacaacttcggtagttttcacattacactcaagaacgtcttt
ctgtatggtaggattttccactacttcttcagagactggttt
CVNS100_RO1
Reverse 1 (SEQ ID NO: 20):
atcaaattcgtttgatgtactgaagtcagaggacgcgcagggaatggata
atcttgcctgcgaagatctaaaaccagtctctgaagaagta
CVNS100_RO2
Reverse 2 (SEQ ID NO: 21):
ctaataaagccacgtataaaccaaatacctggtgtatacgttgtctttgg
agcacaaaaccagttgaaacatcaaattcgtttgatgtact
CVNS100_RO3
Reverse 3 (SEQ ID NO: 22):
ctacacaccctcttttaagaaaggagctaaattgttacataaacctattg
tttggcatgttaacaatgcaactaataaagccacgtataaac

The following amplification primers were used in the method:

| CVNS100 Fwd | |
|---|---|
| Forward (SEQ ID NO: 23): | |
| ATGTCTCCTACAACTTCG | Tm = 56° C. |
| CVNS100 Rev | |
| Reverse (SEQ ID NO: 24): | |
| CTACACACCCTCTTTTAAGA | Tm = 57° C. |

The synthesised polynucleotide has the sequence of SEQ ID NO: 1

(atgtctcctacaacttcggtagttttcacattacactcaagaacgtctt
tctgtatggtaggattttccactacttcttcagagactggttttagatct
tcgcaggcaagattatccattccctgcgcgtcctctgacttcagtacatc
aaacgaatttgatgtttcaactggttttgtgctccaaagacaacgtatac
accaggtatttggtttatacgtggctttattagttgcattgttaacatgc
caaacaataggtttatgtaacaatttagctcctttcttaaaagagggtgt
gtag).

A T7 promoter may be attached by a second PCR followed by purification of the synthesised polynucleotide which is then used as a template for the Invitrogen Transcription Kit (https://www.fishersci.co.uk/shop/products/product/10219104). GFP would be used as a positive control in a parallel reaction. The primers for the second PCR are:

CVNS100_Fwd_T7
Forward (SEQ ID NO: 25):

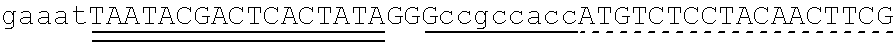

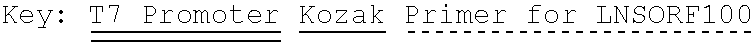

CVNS100_Rev_AAA

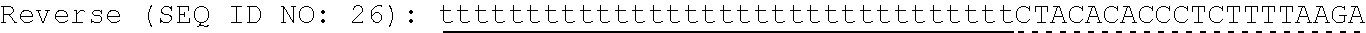

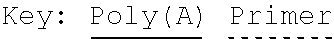

A polynucleotide sequence encoding the ORF11 consensus amino acid sequence (SEQ ID NO: 13) may be used to transfect cells with ORF100 mRNA. Such transfection may provide a beneficial research tool. For example, the peptides displayed on the MHC on the surface of transfected cells may be purified, so that it may be determined if these peptides are from ORF100 and that they match the ORF100 sequences found in the SAR-CoV-2 ligandome. In this case, GFP may be used as a positive control as it should provide an additional readout with the cells fluorescing as well as displaying GFP derived peptides on their MHC class I molecules.

### Example 7-Confirmation of T Cell Epitopes (Peptides) from SARS-COV-2 Virus

### Objective

To confirm the identification of four negative sense peptides in SARS-COV-2 infected cells using synthetic peptide mass spec analysis.

### Method

Four peptides (Table 12) were synthesized and mass spectra data was generated for comparison purpose. We synthesized the targeted peptides and run in the mass spec to compare the spectra generated by experimental (peptides isolated from the SARS-COV-2 infected cells analysis) vs the synthetic peptide to confirm the sequence of peptides.

### Mass Spectrometry Analysis

Samples were analyzed using an Eclipse triploid mass spectrometer coupled to a Dionex Ultimate 3000 RLSCnano System (Thermo Scientific). Samples were loaded on to a fused silica trap column Acclaim PepMap 100, 75 μm×2 cm (ThermoFisher). After washing with 0.1% TFA, the trap column was brought in-line with an analytical column (Nanoease MZ peptide BEH C18, 130A, 1.7 μm, 75 μm×250 mm, Waters) for LC-MS/MS. Peptides were eluted using a segmented linear gradient from 4 to 90% B (A: 0.2% formic acid, B: 0.08% formic acid, 80% ACN): 4-15% B in 5 min, 15-50% B in 50 min, and 50-90% B in 15 min. Mass spectrometry data (CID or HCD mode) was acquired using a data-dependent acquisition procedure with a cyclic series of a full scan from 375-1500 with resolution of 240,000 normalized (AGC) target 250% of normal (1E6), maximum injection time 50 ms. The top S (1 sec) and dynamic exclusion of 30 sec were used for selection of parent ions of charge stage 2-7 for MSMS with intensity threshold at 5E3. The selected ions were transmitted to ion trap with isolation window 1.2 m/z normalized AGC target 200% and fragmented with relative collision energy 35% and scanned in the ion trap with rapid scan rate.

### Peptide Validation by Synthetic Peptides

Synthetic peptides for validating the peptides identified in previous study were obtained from China peptides Co., Ltd (Suzhou, China). The synthetic peptides were then subjected to LC-MS/MS analysis under identical experimental conditions as described above, and their sequences were confirmed based on their MS/MS data. Candidate peptide sequences were confirmed by comparison of their MS/MS spectra with that of their synthetic analogues.

### Results

The data presented in this Example was generated by the confirmation studies using synthetic peptides. The reason being, some peptides (natural or synthetic) have poor fragmentation properties in the mass spec and may generate poor quality spectra, which will categorize the peptide being medium to low hits by the search software. However, if the confirmation studies are run using the synthetic peptides, and if the experimental spectra and the synthetic peptide spectra match, then the peptide sequence can be confirmed even though the software identified as medium to low confidence spectra.

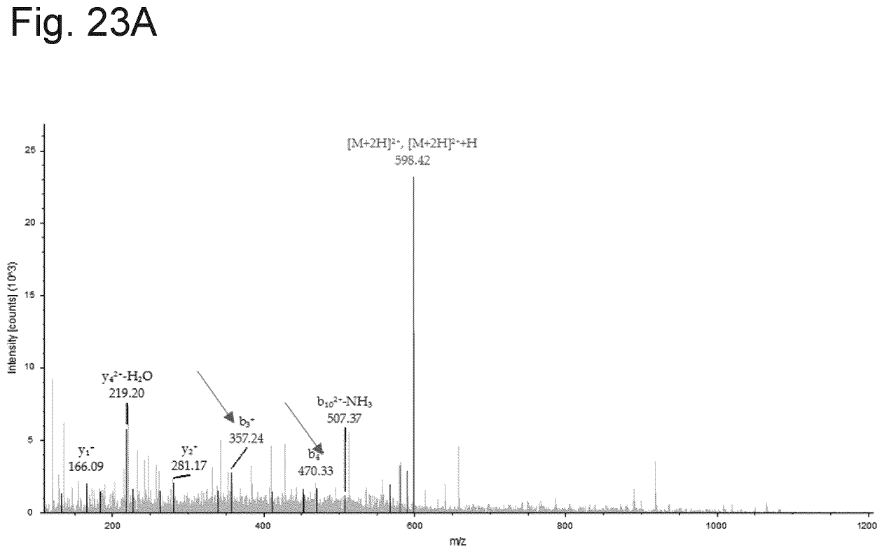

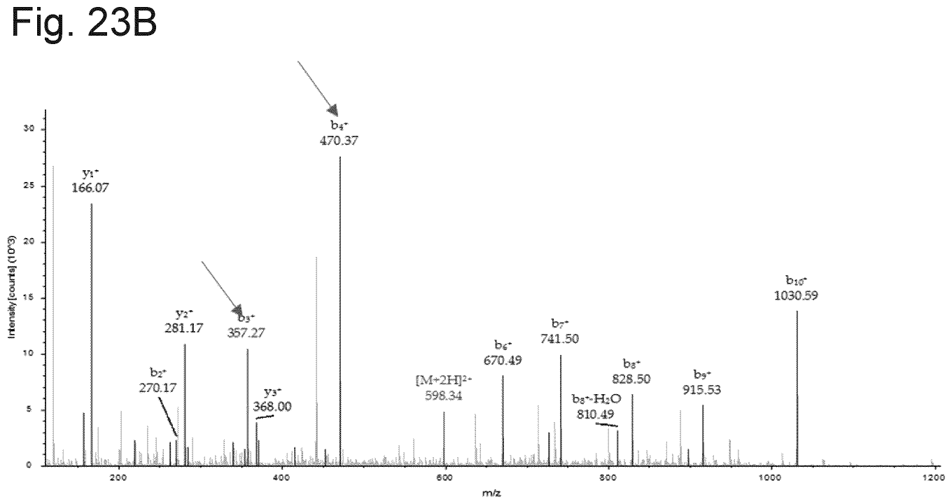

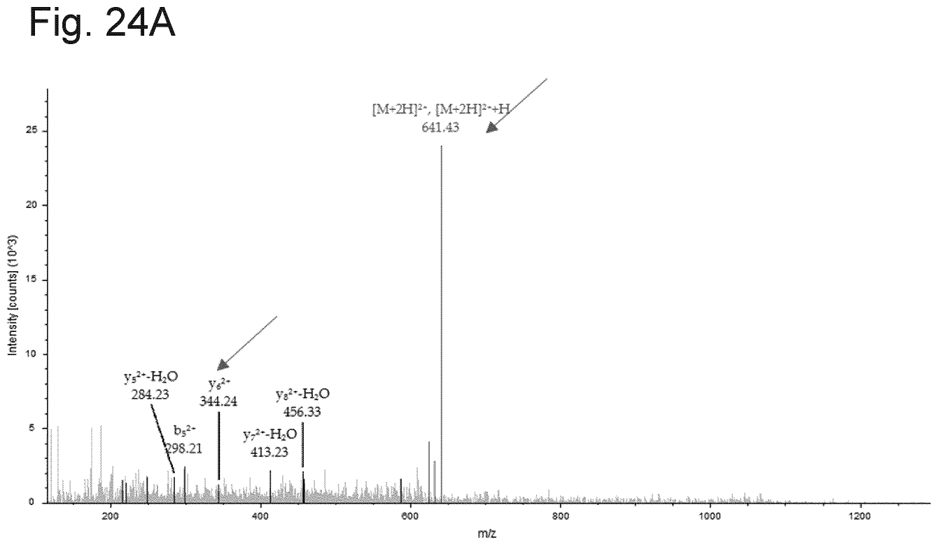

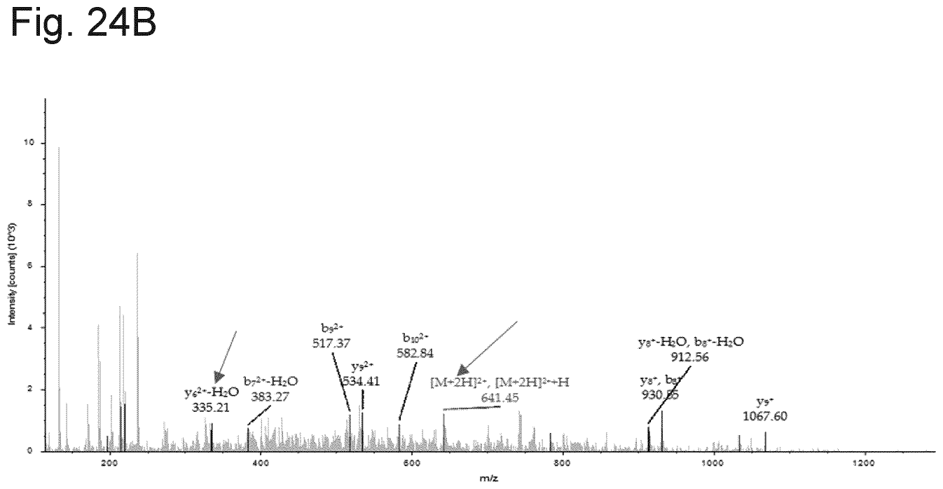

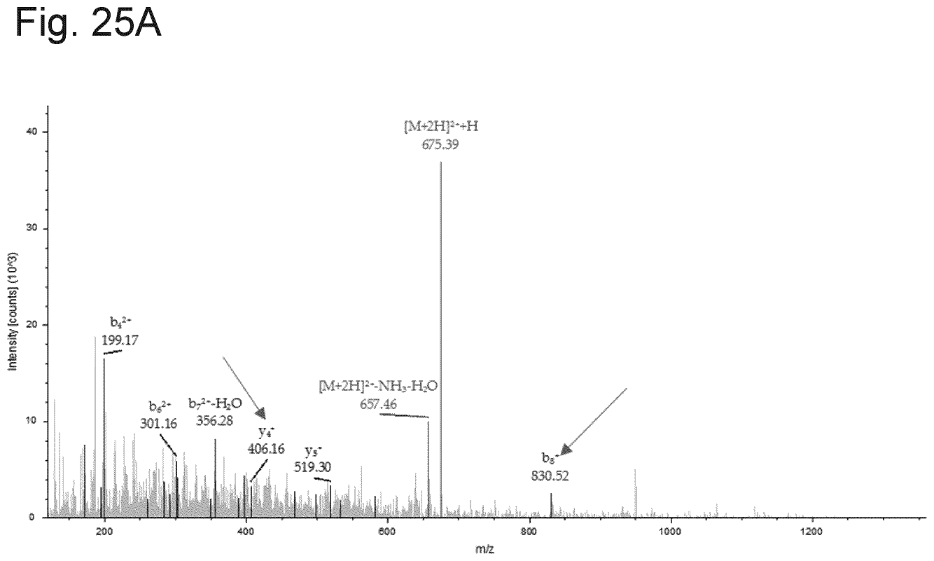

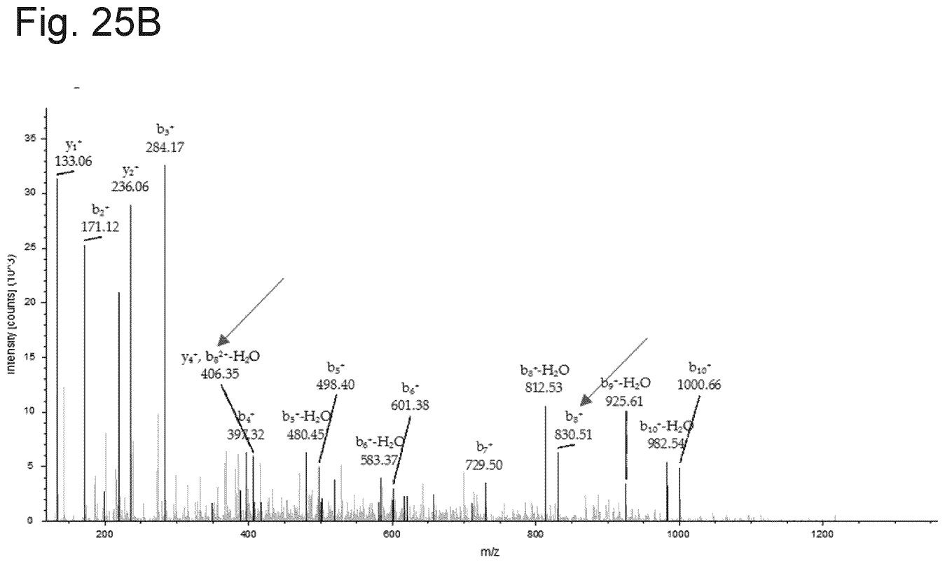

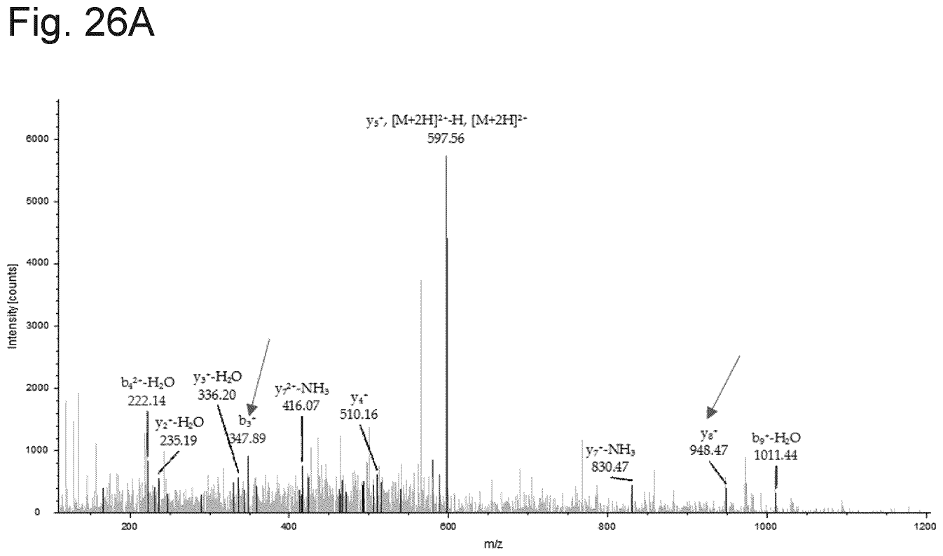

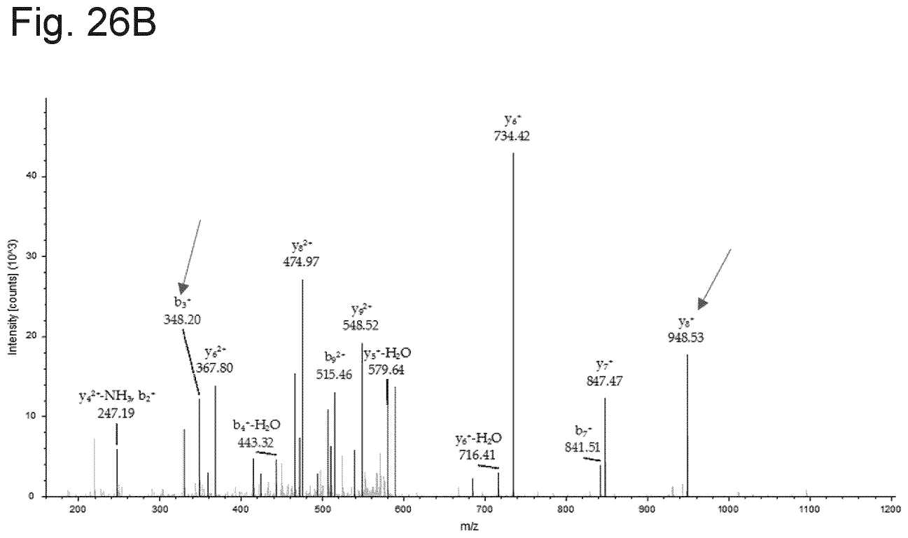

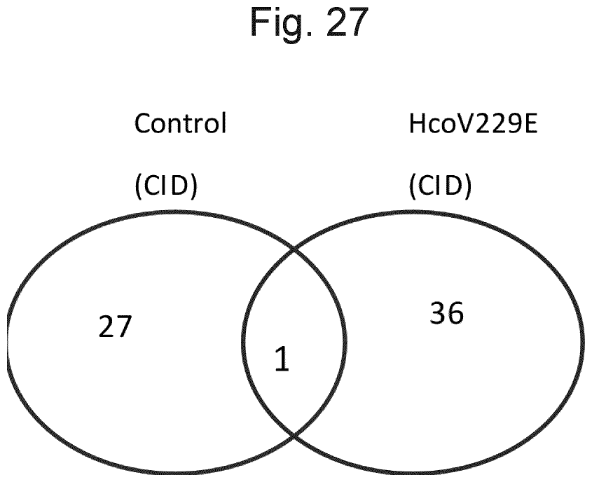

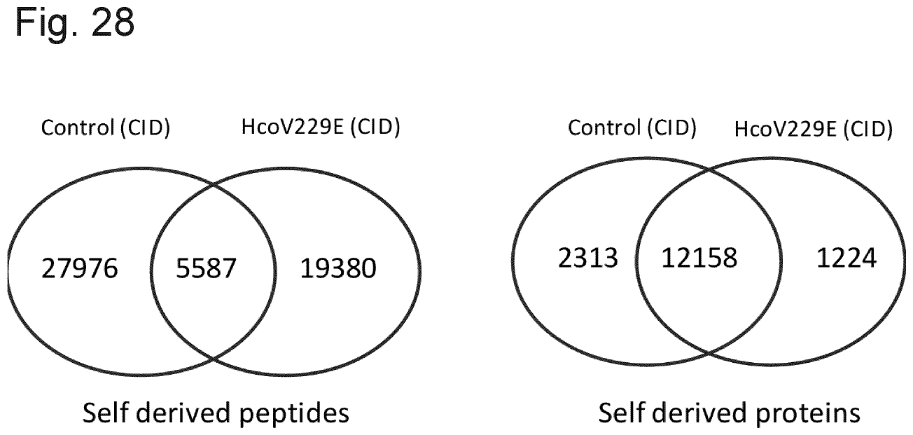

Results are shown in Tables 12 and 13, and in FIGS. 23 to 26.

TABLE 12
List of MHC peptides from SARS-COV-2 infected
cells

| | | SEQ | | | |
|---|---|---|---|---|---|
| | Peptide | ID | | HLA | Confir- |
| Line | Sequence | NO: | Protein | Motif | mation |
| 1 | RLSIPCASSDF | 8 | LNSORF100; | | Yes |
| | | | MN985325_ | | |
| | | | −3_137 | | |
| 2 | TLHSRTSFCMV | 10 | LNSORF100; | A24 | Yes |
| 3 | VALLTCQTIGLCN | 11 | LNSORF100; | A2 | Yes |
| | | | MN985325_ | | |
| | | | −3_137 | | |
| 4 | VFTLHSRTSF | 12 | LNSORF100; | | Yes |

TABLE 13
Mass spec parameter for experimental and
synthetic peptides

| Line | Peptide sequence | XCorr | charge | M/Z |
|---|---|---|---|---|
| 1 | RLSIPCASSDF (cell) | 3.10 | 2 | 598.29547 |
| | RLSIPCASSDF (syn.) | 3.43 | 2 | 598.29218 |
| 2 | TLHSRTSFCMV (cell) | 2.31 | 2 | 649.30481 |
| | TLHSRTSFCMV (syn.) | 2.98 | 2 | 649.3045 |
| 3 | VALLTCQTIGLCN (cell) | 2.02 | 2 | 674.84967 |
| | VALLTCQTIGLCN (syn.) | 4.1 | 2 | 674.85162 |
| 4 | VFTLHSRTSF (cell) | 2.21 | 2 | 597.81268 |
| | VFTLHSRTSF (syn.) | 2.83 | 2 | 597.81702 |

### Example 8

### Introduction

Human coronavirus 229E (HcoV-229E) belongs to one of the seven human coronaviruses which include MERS-COV, SARS-COV-1 and SARS-COV-2. It can infect humans and bats. The current project was designed to identify MHC class I peptides in post-HcoV-229E infection cell pellet and whole cell lysate, post-infection. HepG2 cells were infected, harvested, lysed and MHC class I peptides were extracted to generate peptide sample mixture for mass spectrometry analysis. Additionally, a control sample (mock infection) was collected and processed following the same procedure or infection sample. An ultra high-pressure liquid chromatography (UPLC) system coupled with an Eclipse triploid mass spectrometer was used for peptide separation and identification. One collision mode (CID) were applied in mass spec analysis of infection samples. A data dependent acquisition (DDA) method was implemented for real time sample acquisition. The generated datasets were processed using SEQUEST HT software for the database searches and comparison.

### Methods

### 1 HcoV-229E Infected HepG2 Cell Preparation (Performed by GMU)

HepG2 cells (2×10\hat{ }7 total cells) were grown and infected with HcoV-229E (MOI=0.1) for 24 h.

### 2. HcoV-229E MHC Class I Peptide Discovery Using Mass Spectrometry Sample Analysis

a) Sample preparation: HcoV-229E strain infected HepG2 cells were harvested and washed by PBS once. The cell pellet was treated with 0.1% TFA and the supernatant was collected (provided by GMU). The cell pellet was treated with lysis buffer and processed (provided by GMU) using the immunoproteomic protocol to isolate and purify MHC class I peptides. The two samples (samples from steps 1&2) were pooled, cleaned up and fractionated on an offline HPLC system. Each fraction was analysed on a UPLC-Nano-MS/MS system.
b) DDA mode: Samples were analysed using an Eclipse triploid mass spectrometer coupled to a Dionex Ultimate 3000 RLSCnano System (Thermo Scientific). Samples were loaded on to a fused silica trap column Acclaim PepMap 100, 75 μm×2 cm (ThermoFisher). After washing with 0.1% TFA, the trap column was brought in-line with an analytical column (Nanoease MZ peptide BEH C18, 130A, 1.7 μm, 75 μm×250 mm, Waters) for LC-MS/MS. Peptides were eluted using a segmented linear gradient from gradient 4-15% B in 30 min (where A: 0.2% formic acid, and B: 0.16% formic acid, 80% acetonitrile), 15-25% B in 40 min, 25-50% B in 44 min, and 50-90% B in 11 min. Solution B then returns at 4% for 5 minutes for the next run. Mass spectrometry data (CID mode) was acquired using a data-dependent acquisition procedure with a cyclic series of a full scan from 375-1500 with resolution of 240,000 normalized (AGC) target 250% of normal (1E6), maximum injection time 50 ms. The top S (1 sec) and dynamic exclusion of 30 sec were used for selection of parent ions of charge stage 2-7 for MSMS with intensity threshold at 5E3. The selected ions were transmitted to ion trap with isolation window 1.2 m/z normalized AGC target 200% and fragmented with relative collision energy 35% and scanned in the ion trap with rapid scan rate. Mass spectrometry data was acquired using a data-dependent acquisition procedure with a cyclic series of a full scan from 350-1500 with resolution of 120,000 control (AGC) target 1E6, maximum injection time 100 ms.

### 3. Database Search

Database searching of all raw spectra files was performed using Proteome Discoverer 1.4 (Thermo Fisher Scientific). SEQUEST was used for database searching against HcoV 229E protein sequence databases 1) positive sense translations 2) negative sense translations and 3) IPI human database. Database searching against the corresponding reverse database was also performed to evaluate the false discovery rate (FDR) of peptide/protein identification. The database searching parameters included up to two missed cleavages for no enzyme digestion, precursor mass tolerance 10 ppm, production mass tolerance 0.4 Da, and methionine oxidation as variable modifications. The result from each run was filtered with peptide confidence value as high to obtain FDR less than 1% on peptide level. On the protein level the search parameters included minimum number of peptide 1 for each protein, count only rank 1 peptides, and count peptide only in top scored proteins were applied for all data filtration. In addition, protein grouping was enabled, and strict maximum parsimony principle was applied. Afterwards, manual evaluation was applied into each mass spectra.

### Results

TABLE 14
HcoV-229E ligandome summary

| Sample | Control | HcoV-229E(CID) |
|---|---|---|
| Self-derive peptide | 31719 | 22795 |
| (full list)^{a} | | |
| Self-derive protein | 11638 | 10097 |
| (full list) | | |
| 229E viral peptide | 23 | 37 |
| (full list) | | |

Notes:
^{a}full list generated by pre-screening criteria (confidence-high, amino acid length-8 to 15 aa, rank: 1, XCorr > 1.5 for charge 2, XCorr > 2 for charge 3).

TABLE 15
List of MHC class I peptides from HcoV-229E viral proteins

| SEQ | | Protein source | Viral |
|---|---|---|---|
| ID NO: | Sequence | NCBI ref. seq. | origin |
| 27 | AMLKCVAFCDE | NCBI ref. seq. NP_073549.1 | 229E |
| 28 | ANGCSTIAQAV | NCBI ref. seq. NP_073549.1 | 229E |
| 29 | AQGVFGVNM | NCBI ref. seq. NP_073549.1 | 229E |
| 30 | ARLEPCNGTDID | NCBI ref. seq. NP_073549.1 | 229E |
| 31 | AVTTGDVKIM | NCBI ref. seq. NP_073549.1 | 229E |
| 32 | DIVVVDEVSMCTNYD | NCBI ref. seq. NP_073549.1 | 229E |
| 33 | DSLCAKAVTAY | NCBI ref. seq. NP_073549.1 | 229E |
| 34 | EDFLNMDIGVFIQ | NCBI ref. seq. NP_073549.1 | 229E |
| 35 | EVNADIVVVDEVSMC | NCBI ref. seq. NP_073549.1 | 229E |
| 36 | FVGADGELPV | NCBI ref. seq. NP_073549.1 | 229E |
| 37 | FVKSICNSAVAV | NCBI ref. seq. NP_073549.1 | 229E |
| 38 | FYCTNNTLVSGDAHI | NCBI ref. seq. NP_073549.1 | 229E |
| 39 | GAKVVNANVLTK | NCBI ref. seq. NP_073549.1 | 229E |
| 40 | GYIADISAF | NCBI ref. seq. NP_073549.1 | 229E |
| 41 | IACSKSARLKRFPVN | NCBI ref. seq. NP_073549.1 | 229E |
| 42 | IADFLAGSSDV | NCBI ref. seq. NP_073549.1 | 229E |
| 43 | IFAQTSDTA | NCBI ref. seq. NP_073549.1 | 229E |
| 44 | IVQMIADFLA | NCBI ref. seq. NP_073549.1 | 229E |
| 45 | KFLNAFDVFVTAIQ | NCBI ref. seq. NP_073549.1 | 229E |
| 46 | LIAGKLLPPV | NC_002645_Human_coronavirus_ | 229E |
| | | 229E_Translation_RF-1_24 | |
| 47 | LNCALGAFAIFCC | NCBI ref. seq. NP_073549.1 | 229E |
| 48 | MHGVTLKI | NCBI ref. seq. NP_073549.1 | 229E |
| 49 | MKVKATKGEGDGGI | NCBI ref. seq. NP_073549.1 | 229E |
| 50 | NAMLKCVAF | NCBI ref. seq. NP_073549.1 | 229E |
| 51 | NEADYRCACYA | NCBI ref. seq. NP_073549.1 | 229E |
| 52 | PNLNLGILQVT | NC_002645_Human_coronavirus_ | 229E |
| | | 229E_Translation_RF + 1_20 | |
| 53 | PSLVMPPSPSPLV | NC_002645_Human_coronavirus_ | 229E |
| | | 229E_Translation_RF-3_42 | |
| 54 | QAAAAMYKEARAVN | NCBI ref. seq. NP_073549.1 | 229E |
| 55 | QTSQALQTVATALNK | NCBI ref. seq. NP_073549.1 | 229E |
| 56 | SEISANGCSTIAQA | NCBI ref. seq. NP_073549.1 | 229E |
| 57 | SNFNTLFATTIPN | NCBI ref. seq. NP_073549.1 | 229E |
| 58 | TIQGPPGSGKS | NCBI ref. seq. NP_073549.1 | 229E |
| 59 | TNVPLQVGFSNG | NCBI ref. seq. NP_073549.1 | 229E |
| 60 | VGGTIQIL | NCBI ref. seq. NP_073549.1 | 229E |
| 61 | VLFSATAVKTGGK | NCBI ref. seq. NP_073549.1 | 229E |
| 62 | VLNNGFGGKQI | NCBI ref. seq. NP_073549.1 | 229E |
| 63 | VTSGLGTVDADY | NCBI ref. seq. NP_073549.1 | 229E |

SEQ ID NOs: 46 (LIAGKLLPPV) and 53 (PSLVMPPSPSPLV) are reverse peptides from the common cold coronavirus 229E. That is, SEQ ID NOs: 46 (LIAGKLLPPV) and 53 (PSLVMPPSPSPLV) are epitopes from a polypeptide encoded by an open reading frame (ORF) encoded by at least part of the genome of a 229E common cold coronavirus in the opposite sense to positive sense RNA capable of translation.

By running the SYFPEITHI epitope binding prediction program, SEQ ID NO: 46 (LAIGKLLPPV); was predicted to have the following HLA binding: A2: 29.

Sequence listing
SEQ ID NO: 1
angncnccnacaacnncggnagnnnncacannacacncaagaacgncnnncngnanggnaggannnnccacnacnnc
nncagagacnggnnnnagancnncgcaggcaagannanccanncccngcgcgnccncngacnncagnacancaaacg
aannngangnnncaacnggnnnngngcnccaaagacaacgnanacaccaggnannnggnnnanacgnggcnnnann
agnngcanngnnaacangccaaacaanaggnnnangnaacaannnagcnccnnncnnaaaagagggngngnag
SEQ ID NO: 2
ALLTCQTIGLCN
SEQ ID NO: 3
LHSRTSFCMVGFS
SEQ ID NO: 4
LSIPCASSDFST
SEQ ID NO: 5
LTCQTIGLCNNLAPF
SEQ ID NO: 6
LYVALLVALLTCQ
SEQ ID NO: 7
QTIGLCNNLAP
SEQ ID NO: 8
RLSIPCASSDF
SEQ ID NO: 9
TCQTIGLCNNLAPFL
SEQ ID NO: 10
TLHSRTSFCMV
SEQ ID NO: 11
VALLTCQTIGLCN
SEQ ID NO: 12
VFTLHSRTSF
SEQ ID NO: 13
MSPTTSVVFTLHSRTSFCMVGFSTTSSETGFRSSQARLSIPCASSDFSTSNEFDVSTG
FVLQRQRIHQVFGLYVALLVALLTCQTIGLCNNLAPFLKEGV
SEQ ID NO: 14
MSPTTSVVFTLHSRTSFCMVGFSTTSSETGFRSSQARLSIPCASSDFSTSNEFDVSTG
FVLQRQRIHQVFGLYVALLVALLTCQTIGLCNNLAPFLKEGV
SEQ ID NO: 15
MSPTTSVVFTLHSRTSFCMVGFSTTSSETGFRSSQARLSIPCASSDFSTSNEFDVSTG
FVLQRQRIHQVFGLYVALLVALLTCQTIGLCNNLAPFLKEGV
SEQ ID NO: 16
MSPTTSVVFTLHSRMSFCMVGFSTTSSETGFRSSQARLSIPCVSSDFSTSKEFDVSTG
FVFQRQRIHQVFGLYVALLVALLTCQTIGLCNNLAPFLKEGV
SEQ ID NO: 17
MSPTTSVVFTLHSRMSFCMVGFSTTSSETGFRTSQARLSIPCVSPNFSASKEFDVST
GFVLQRQRMHQIFGLYVALLVALLTCQTIGLCNNLAPFLKEGV
SEQ ID NO: 18
MSPTTSVVFTLHSRMSFCMVGFSTTSSETGFRTSQARLSIPCVSPNSSASKEFDVST
GFVLQRQRMHQIFGLYVALLVALLTCQTIGLCSNLAPFLKEGV
SEQ ID NO: 19
atgtctcctacaacttcggtagttttcacattacactcaagaacgtctttctgtatggtaggattttccactcttcttcagagactggtt
SEQ ID NO: 20
atcaaattcgtttgatgtactgaagtcagaggacgcgcagggaatggataatcttgcctgcgaagatctaaaaccagtctctgaaga
agta
SEQ ID NO: 21
ctaataaagccactataaaccaaatacctggtgtatacgttgtctttggagcacaaaaccagttgaaacatcaaattcgtttgatgta
ct
SEQ ID NO: 22
ctacacaccctcttttaagaaaggagctaaattgttacataaacctattgtttggcatgttaacaatgcaactaataaagccacgtataa
ac
SEQ ID NO: 23
ATGTCTCCTACAACTTCG
SEQ ID NO: 24
CTACACACCCTCTTTTAAGA
SEQ ID NO: 25

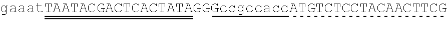

SEQ ID NO: 26

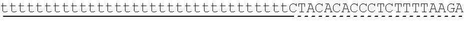

SEQ ID NO: 27
AMLKCVAFCDE
SEQ ID NO: 28
ANGSCTIAQAV
SEQ ID NO: 29
AQGVFGVNM
SEQ ID NO: 30
ARLEPCNGTDID
SEQ ID NO: 31
AVTTGDVKIM
SEQ ID NO: 32
DIVVVDEVSMCTNYD
SEQ ID NO: 33
DSLCAKAVTAY
SEQ ID NO: 34
EDFLNMDIGVFIQ
SEQ ID NO: 35
EVNADIVVVDEVSMC
SEQ ID NO: 36
FVGADGELPV
SEQ ID NO: 37
FVKSICNSAVAV
SEQ ID NO: 38
FYCTNNTLVSGDAHI
SEQ ID NO: 39
GAKVVNANVLTK
SEQ ID NO: 40
GYIADISAF
SEQ ID NO: 41
IACSKSARLKRFPVN
SEQ ID NO: 42
IADFLAGSSDV
SEQ ID NO: 43
IFAQTSDTA
SEQ ID NO: 44
IVQMIADFLA
SEQ ID NO: 45
KFLNAFDVFVTAIQ
SEQ ID NO: 46
LIAGKLLPPV
SEQ ID NO: 47
LNCALGAFAIFCC
SEQ ID NO: 48
MHGVTLKI
SEQ ID NO: 49
MKVKATKGEEGDGGI
SEQ ID NO: 50
NAMLKCVAF
SEQ ID NO: 51
NEADYRCACYA
SEQ ID NO: 52
PNLNLGILQVT
SEQ ID NO: 53
PSLVMPPSPSPLV
SEQ ID NO: 54
QAAAAMYKEARAVN
SEQ ID NO: 55
QTSQALQTVATALNK
SEQ ID NO: 56
SEISANGCSTIAQA
SEQ ID NO: 57
SNFNTLFATTIPN
SEQ ID NO: 58
TIQGPPGSGKS
SEQ ID NO: 59
TNVPLQVGFSNG
SEQ ID NO: 60
VGGTIQIL
SEQ ID NO: 61
VLFSATAVKTGGK
SEQ ID NO: 62
VLNNGFGGKQI
SEQ ID NO: 63
VTSGLGTVDADY

1. An immunogenic peptide comprising an epitope from a polypeptide encoded by an open reading frame (ORF) encoded by at least part of the reverse complement of the genome of a coronavirus.

2. The immunogenic peptide of claim 1, wherein the polypeptide enhances the fitness of the coronavirus in humans.

3. (canceled)

4. The immunogenic peptide of claim 1, wherein the coronavirus is SARS-COV-2, or a common cold virus, optionally 299E.

5. The immunogenic peptide of claim 1, wherein the ORF is encoded by at least part of the reverse complement of the orflab gene.

6. The immunogenic peptide of claim 5, wherein the ORF is about 100 codons in length.

7. The immunogenic peptide of claim 1, wherein the ORF comprises the sequence of SEQ ID NO. 1 or a variant thereof, or encodes a polypeptide comprising the sequence of SEQ ID NO: 13 or a variant thereof.

8. The immunogenic peptide of claim 1, wherein the epitope is a CD8+ T cell epitope, a CD4+T cell epitope, or a B cell epitope.

9. The immunogenic peptide of claim 1, wherein the epitope is conserved between two or more coronaviruses, optionally wherein the epitope is conserved between (i) two or more human coronaviruses, (ii) two or more animal coronaviruses, or (iii) one or more human coronaviruses and one or more animal coronaviruses.

10. The immunogenic peptide of claim 1, wherein the epitope is conserved between SARS-COV-2 and one or more of (a) SARS-COV-1, (b)229E, (c) NL63, (d) OC43, (e) HKU1 and (f) MERS-COV.

11. The immunogenic peptide of claim 1, wherein the epitope is present in a polypeptide encoded by a predicted ORF of about 100 codons in length in one or more of (a) SARS-COV-1, (b)229E, (c) NL63, (d) OC43, (e) HKU1 and (f) MERS-COV.

12. The immunogenic peptide of claim 1, comprising one or more of the peptides set out in SEQ ID NOs: 2 to 12.

13. (canceled)

14. A pharmaceutical composition comprising the immunogenic peptide of claim 1, or a polynucleotide encoding the immunogenic peptide of claim 1.

15. The pharmaceutical composition of claim 14, which comprises:
(a) two or more immunogenic peptides, optionally wherein the two or more immunogenic peptides are immunogenic peptides according to claim 1; or
(b) two or more polynucleotides, optionally wherein the two or more polynucleotides are polynucleotides encoding an immunogenic peptide according to claim 1.

16. The pharmaceutical composition of claim 14, which comprises:
(a) at least one immunogenic peptide that interacts with at least two different HLA supertypes, or at least two immunogenic peptides that each interact with a different HLA supertype; or
(b) at least one polynucleotide that encodes an immunogenic peptide that interacts with at least two different HLA supertypes, or at least two polynucleotides that each encode a different immunogenic peptide wherein each immunogenic peptide interacts with a different HLA supertype.

17. A method of preventing and/or treating a coronaviral infection, comprising administering the pharmaceutical composition of claim 14 to an individual.

18. (canceled)

19. (canceled)

20. The method of claim 17, wherein the coronaviral infection is caused by a zoonotic virus.

21. The method of claim 17, wherein the individual is human.

22. The method of claim 17, wherein the coronaviral infection is an endemic viral infection, a seasonal viral infection, or a pandemic viral infection.

23. (canceled)

24. (canceled)

25. (canceled)

26. (canceled)

27. The pharmaceutical composition of claim 14, wherein the immunogenic peptide is attached to a gold nanoparticle.

28. The pharmaceutical composition of claim 27, wherein the gold nanoparticle is coated with alpha-galactose and/or beta-GlcNHAc.


SEQUENCE LISTING (63 sequences)

SEQ ID NO: 1 - DNA, 303 bp, Artificial Sequence
  SARS-CoV-2
  misc_feature (2)..(2): n is t or u
  misc_feature (4)..(4): n is t or u
  misc_feature (6)..(6): n is t or u
  misc_feature (9)..(9): n is t or u
  misc_feature (15)..(16): n is t or u
  misc_feature (20)..(20): n is t or u
  misc_feature (23)..(26): n is t or u
  misc_feature (31)..(32): n is t or u
  misc_feature (37)..(37): n is t or u
  misc_feature (46)..(46): n is t or u
  misc_feature (48)..(50): n is t or u
  misc_feature (52)..(52): n is t or u
  misc_feature (54)..(54): n is t or u
  misc_feature (56)..(56): n is t or u
  misc_feature (59)..(59): n is t or u
  misc_feature (64)..(67): n is t or u
  misc_feature (72)..(72): n is t or u
  misc_feature (75)..(76): n is t or u
  misc_feature (78)..(79): n is t or u
  misc_feature (87)..(87): n is t or u
  misc_feature (90)..(93): n is t or u
  misc_feature (97)..(97): n is t or u
  misc_feature (99)..(100): n is t or u
  misc_feature (112)..(113): n is t or u
  misc_feature (115)..(115): n is t or u
  misc_feature (119)..(120): n is t or u
  misc_feature (124)..(124): n is t or u
  misc_feature (130)..(130): n is t or u
  misc_feature (133)..(133): n is t or u
  misc_feature (135)..(135): n is t or u
  misc_feature (139)..(140): n is t or u
  misc_feature (144)..(144): n is t or u
  misc_feature (148)..(148): n is t or u
  misc_feature (157)..(159): n is t or u
  misc_feature (162)..(162): n is t or u
  misc_feature (164)..(166): n is t or u
  misc_feature (171)..(171): n is t or u
  misc_feature (174)..(177): n is t or u
  misc_feature (179)..(179): n is t or u
  misc_feature (182)..(182): n is t or u
  misc_feature (195)..(195): n is t or u
  misc_feature (197)..(197): n is t or u
  misc_feature (206)..(206): n is t or u
  misc_feature (208)..(210): n is t or u
  misc_feature (213)..(215): n is t or u
  misc_feature (217)..(217): n is t or u
  misc_feature (221)..(221): n is t or u
  misc_feature (225)..(227): n is t or u
  misc_feature (229)..(230): n is t or u
  misc_feature (233)..(234): n is t or u
  misc_feature (238)..(239): n is t or u
  misc_feature (241)..(242): n is t or u
  misc_feature (247)..(247): n is t or u
  misc_feature (257)..(257): n is t or u
  misc_feature (261)..(263): n is t or u
  misc_feature (265)..(265): n is t or u
  misc_feature (267)..(267): n is t or u
  misc_feature (273)..(275): n is t or u
  misc_feature (279)..(279): n is t or u
  misc_feature (282)..(284): n is t or u
  misc_feature (286)..(287): n is t or u
  misc_feature (297)..(297): n is t or u
  misc_feature (299)..(299): n is t or u
  misc_feature (301)..(301): n is t or u
angncnccnacaacnncggnagnnnncacannacacncaagaacgncnnncngnanggnaggannnnccacnacnncnncagagacnggnnnnagancnncgcaggcaagannanccanncccngcgcgnccncngacnncagnacancaaacgaannngangnnncaacnggnnnngngcnccaaagacaacgnanacaccaggnannnggnnnanacgnggcnnnannagnngcanngnnaacangccaaacaanaggnnnangnaacaannnagcnccnnncnnaaaagagggngngnag

SEQ ID NO: 2 - protein, 12 aa, Artificial Sequence
  MHC class I peptides from SCV2 viral proteins
ALLTCQTIGLCN

SEQ ID NO: 3 - protein, 13 aa, Artificial Sequence
  MHC class I peptides from SCV2 viral proteins
LHSRTSFCMVGFS

SEQ ID NO: 4 - protein, 12 aa, Artificial Sequence
  MHC class I peptides from SCV2 viral proteins
LSIPCASSDFST

SEQ ID NO: 5 - protein, 15 aa, Artificial Sequence
  MHC class I peptides from SCV2 viral proteins
LTCQTIGLCNNLAPF

SEQ ID NO: 6 - protein, 13 aa, Artificial Sequence
  MHC class I peptides from SCV2 viral proteins
LYVALLVALLTCQ

SEQ ID NO: 7 - protein, 11 aa, Artificial Sequence
  MHC class I peptides from SCV2 viral proteins
QTIGLCNNLAP

SEQ ID NO: 8 - protein, 11 aa, Artificial Sequence
  MHC class I peptides from SCV2 viral proteins
RLSIPCASSDF

SEQ ID NO: 9 - protein, 15 aa, Artificial Sequence
  MHC class I peptides from SCV2 viral proteins
TCQTIGLCNNLAPFL

SEQ ID NO: 10 - protein, 11 aa, Artificial Sequence
  MHC class I peptides from SCV2 viral proteins
TLHSRTSFCMV

SEQ ID NO: 11 - protein, 13 aa, Artificial Sequence
  MHC class I peptides from SCV2 viral proteins
VALLTCQTIGLCN

SEQ ID NO: 12 - protein, 10 aa, Artificial Sequence
  MHC class I peptides from SCV2 viral proteins
VFTLHSRTSF

SEQ ID NO: 13 - protein, 100 aa, Artificial Sequence
  SARS-CoV-2 orf1ab
MSPTTSVVFTLHSRTSFCMVGFSTTSSETGFRSSQARLSIPCASSDFSTSNEFDVSTGFVLQRQRIHQVFGLYVALLVALLTCQTIGLCNNLAPFLKEGV

SEQ ID NO: 14 - protein, 100 aa, Artificial Sequence; identical to SEQ ID NO: 13
  SARS-CoV-2 WIV04 isolate

SEQ ID NO: 15 - protein, 100 aa, Artificial Sequence; identical to SEQ ID NO: 13
  SARS-CoV-2 Wuhan-Hu-1 isolate

SEQ ID NO: 16 - protein, 100 aa, Artificial Sequence
  SARS-CoV-2
MSPTTSVVFTLHSRMSFCMVGFSTTSSETGFRSSQARLSIPCVSSDFSTSKEFDVSTGFVFQRQRIHQVFGLYVALLVALLTCQTIGLCNNLAPFLKEGV

SEQ ID NO: 17 - protein, 100 aa, Artificial Sequence
  SARS-CoV-2
MSPTTSVVFTLHSRMSFCMVGFSTTSSETGFRTSQARLSIPCVSPNFSASKEFDVSTGFVLQRQRMHQIFGLYVALLVALLTCQTIGLCNNLAPFLKEGV

SEQ ID NO: 18 - protein, 100 aa, Artificial Sequence
  SARS-CoV-2
MSPTTSVVFTLHSRMSFCMVGFSTTSSETGFRTSQARLSIPCVSPNSSASKEFDVSTGFVLQRQRMHQIFGLYVALLVALLTCQTIGLCSNLAPFLKEGV

SEQ ID NO: 19 - DNA, 92 bp, Artificial Sequence
  CVNS100 - Forward Oligo 1
atgtctcctacaacttcggtagttttcacattacactcaagaacgtctttctgtatggtaggattttccactacttcttcagagactggttt

SEQ ID NO: 20 - DNA, 91 bp, Artificial Sequence
  CVNS100 - Reverse Oligo 1
atcaaattcgtttgatgtactgaagtcagaggacgcgcagggaatggataatcttgcctgcgaagatctaaaaccagtctctgaagaagta

SEQ ID NO: 21 - DNA, 91 bp, Artificial Sequence
  CVNS100 - Reverse Oligo 2
ctaataaagccacgtataaaccaaatacctggtgtatacgttgtctttggagcacaaaaccagttgaaacatcaaattcgtttgatgtact

SEQ ID NO: 22 - DNA, 92 bp, Artificial Sequence
  CVNS100 - Reverse Oligo 3
ctacacaccctcttttaagaaaggagctaaattgttacataaacctattgtttggcatgttaacaatgcaactaataaagccacgtataaac

SEQ ID NO: 23 - DNA, 18 bp, Artificial Sequence
  CVNS100 - Forward
atgtctcctacaacttcg

SEQ ID NO: 24 - DNA, 20 bp, Artificial Sequence
  CVNS100 - Reverse
ctacacaccctcttttaaga

SEQ ID NO: 25 - DNA, 51 bp, Artificial Sequence
  CVNS100 - Forward_T7
gaaattaatacgactcactatagggccgccaccatgtctcctacaacttcg

SEQ ID NO: 26 - DNA, 53 bp, Artificial Sequence
  CVNS100 - Reverse-AAA
tttttttttttttttttttttttttttttttttctacacaccctcttttaaga

SEQ ID NO: 27 - protein, 11 aa, Artificial Sequence
  MHC class I peptide from HcoV-229E viral protein
AMLKCVAFCDE

SEQ ID NO: 28 - protein, 11 aa, Artificial Sequence
  MHC class I peptide from HcoV-229E viral protein
ANGCSTIAQAV

SEQ ID NO: 29 - protein, 9 aa, Artificial Sequence
  MHC class I peptide from HcoV-229E viral protein
AQGVFGVNM

SEQ ID NO: 30 - protein, 12 aa, Artificial Sequence
  MHC class I peptide from HcoV-229E viral protein
ARLEPCNGTDID

SEQ ID NO: 31 - protein, 10 aa, Artificial Sequence
  MHC class I peptide from HcoV-229E viral protein
AVTTGDVKIM

SEQ ID NO: 32 - protein, 15 aa, Artificial Sequence
  MHC class I peptide from HcoV-229E viral protein
DIVVVDEVSMCTNYD

SEQ ID NO: 33 - protein, 11 aa, Artificial Sequence
  MHC class I peptide from HcoV-229E viral protein
DSLCAKAVTAY

SEQ ID NO: 34 - protein, 13 aa, Artificial Sequence
  MHC class I peptide from HcoV-229E viral protein
EDFLNMDIGVFIQ

SEQ ID NO: 35 - protein, 15 aa, Artificial Sequence
  MHC class I peptide from HcoV-229E viral protein
EVNADIVVVDEVSMC

SEQ ID NO: 36 - protein, 10 aa, Artificial Sequence
  MHC class I peptide from HcoV-229E viral protein
FVGADGELPV

SEQ ID NO: 37 - protein, 12 aa, Artificial Sequence
  MHC class I peptide from HcoV-229E viral protein
FVKSICNSAVAV

SEQ ID NO: 38 - protein, 15 aa, Artificial Sequence
  MHC class I peptide from HcoV-229E viral protein
FYCTNNTLVSGDAHI

SEQ ID NO: 39 - protein, 12 aa, Artificial Sequence
  MHC class I peptide from HcoV-229E viral protein
GAKVVNANVLTK

SEQ ID NO: 40 - protein, 9 aa, Artificial Sequence
  MHC class I peptide from HcoV-229E viral protein
GYIADISAF

SEQ ID NO: 41 - protein, 15 aa, Artificial Sequence
  MHC class I peptide from HcoV-229E viral protein
IACSKSARLKRFPVN

SEQ ID NO: 42 - protein, 11 aa, Artificial Sequence
  MHC class I peptide from HcoV-229E viral protein
IADFLAGSSDV

SEQ ID NO: 43 - protein, 9 aa, Artificial Sequence
  MHC class I peptide from HcoV-229E viral protein
IFAQTSDTA

SEQ ID NO: 44 - protein, 10 aa, Artificial Sequence
  MHC class I peptide from HcoV-229E viral protein
IVQMIADFLA

SEQ ID NO: 45 - protein, 14 aa, Artificial Sequence
  MHC class I peptide from HcoV-229E viral protein
KFLNAFDVFVTAIQ

SEQ ID NO: 46 - protein, 10 aa, Artificial Sequence
  MHC class I peptide from HcoV-229E viral protein
LIAGKLLPPV

SEQ ID NO: 47 - protein, 13 aa, Artificial Sequence
  MHC class I peptide from HcoV-229E viral protein
LNCALGAFAIFCC

SEQ ID NO: 48 - protein, 8 aa, Artificial Sequence
  MHC class I peptide from HcoV-229E viral protein
MHGVTLKI

SEQ ID NO: 49 - protein, 14 aa, Artificial Sequence
  MHC class I peptide from HcoV-229E viral protein
MKVKATKGEGDGGI

SEQ ID NO: 50 - protein, 9 aa, Artificial Sequence
  MHC class I peptide from HcoV-229E viral protein
NAMLKCVAF

SEQ ID NO: 51 - protein, 11 aa, Artificial Sequence
  MHC class I peptide from HcoV-229E viral protein
NEADYRCACYA

SEQ ID NO: 52 - protein, 11 aa, Artificial Sequence
  MHC class I peptide from HcoV-229E viral protein
PNLNLGILQVT

SEQ ID NO: 53 - protein, 13 aa, Artificial Sequence
  MHC class I peptide from HcoV-229E viral protein
PSLVMPPSPSPLV

SEQ ID NO: 54 - protein, 14 aa, Artificial Sequence
  MHC class I peptide from HcoV-229E viral protein
QAAAAMYKEARAVN

SEQ ID NO: 55 - protein, 15 aa, Artificial Sequence
  MHC class I peptide from HcoV-229E viral protein
QTSQALQTVATALNK

SEQ ID NO: 56 - protein, 14 aa, Artificial Sequence
  MHC class I peptide from HcoV-229E viral protein
SEISANGCSTIAQA

SEQ ID NO: 57 - protein, 13 aa, Artificial Sequence
  MHC class I peptide from HcoV-229E viral protein
SNFNTLFATTIPN

SEQ ID NO: 58 - protein, 11 aa, Artificial Sequence
  MHC class I peptide from HcoV-229E viral protein
TIQGPPGSGKS

SEQ ID NO: 59 - protein, 12 aa, Artificial Sequence
  MHC class I peptide from HcoV-229E viral protein
TNVPLQVGFSNG

SEQ ID NO: 60 - protein, 8 aa, Artificial Sequence
  MHC class I peptide from HcoV-229E viral protein
VGGTIQIL

SEQ ID NO: 61 - protein, 13 aa, Artificial Sequence
  MHC class I peptide from HcoV-229E viral protein
VLFSATAVKTGGK

SEQ ID NO: 62 - protein, 11 aa, Artificial Sequence
  MHC class I peptide from HcoV-229E viral protein
VLNNGFGGKQI

SEQ ID NO: 63 - protein, 12 aa, Artificial Sequence
  MHC class I peptide from HcoV-229E viral protein
VTSGLGTVDADY